SEQUENCE LISTING (315 sequences)

SEQ ID NO: 1 - DNA, 18 bp, synthetic construct
  misc_feature 1..18: note = Description of Artificial Sequence: Synthetic primer
  source 1..18: mol_type = other DNA
tcttccaacttggaaggg

SEQ ID NO: 2 - DNA, 19 bp, synthetic construct
  misc_feature 1..19: note = Description of Artificial Sequence: Synthetic primer
  source 1..19: mol_type = other DNA
ggtcatctgctgaaatcac

SEQ ID NO: 3 - DNA, 18 bp, synthetic construct
  misc_feature 1..18: note = Description of Artificial Sequence: Synthetic primer
  source 1..18: mol_type = other DNA
tcttccaacttgcaaggg

SEQ ID NO: 4 - DNA, 22 bp, synthetic construct
  misc_feature 1..22: note = Description of Artificial Sequence: Synthetic primer
  source 1..22: mol_type = other DNA
ggtcatctgctgtaatcacttg

SEQ ID NO: 5 - DNA, 18 bp, synthetic construct
  misc_feature 1..18: note = Description of Artificial Sequence: Synthetic primer
  source 1..18: mol_type = other DNA
gggtcatcctgtttccag

SEQ ID NO: 6 - DNA, 22 bp, synthetic construct
  misc_feature 1..22: note = Description of Artificial Sequence: Synthetic primer
  source 1..22: mol_type = other DNA
tacctaaggacctgtgtagagg

SEQ ID NO: 7 - DNA, 18 bp, synthetic construct
  misc_feature 1..18: note = Description of Artificial Sequence: Synthetic primer
  source 1..18: mol_type = other DNA
tggcctcccaaagtactg

SEQ ID NO: 8 - DNA, 20 bp, synthetic construct
  misc_feature 1..20: note = Description of Artificial Sequence: Synthetic primer
  source 1..20: mol_type = other DNA
tcctctttctatgtcccagg

SEQ ID NO: 9 - DNA, 19 bp, synthetic construct
  misc_feature 1..19: note = Description of Artificial Sequence: Synthetic primer
  source 1..19: mol_type = other DNA
ccaacttggaagggagaac

SEQ ID NO: 10 - DNA, 18 bp, synthetic construct
  misc_feature 1..18: note = Description of Artificial Sequence: Synthetic primer
  source 1..18: mol_type = other DNA
aaaatgccgtctacaccc

SEQ ID NO: 11 - DNA, 19 bp, synthetic construct
  misc_feature 1..19: note = Description of Artificial Sequence: Synthetic primer
  source 1..19: mol_type = other DNA
ccaggtcacctagagcaac

SEQ ID NO: 12 - DNA, 19 bp, synthetic construct
  misc_feature 1..19: note = Description of Artificial Sequence: Synthetic primer
  source 1..19: mol_type = other DNA
tgtccatttcatatgacgg

SEQ ID NO: 13 - DNA, 21 bp, synthetic construct
  misc_feature 1..21: note = Description of Artificial Sequence: Synthetic primer
  source 1..21: mol_type = other DNA
ttatcaaaagtggagcagttc

SEQ ID NO: 14 - DNA, 19 bp, synthetic construct
  misc_feature 1..19: note = Description of Artificial Sequence: Synthetic primer
  source 1..19: mol_type = other DNA
cagctatccatttccacgg

SEQ ID NO: 15 - DNA, 24 bp, synthetic construct
  misc_feature 1..24: note = Description of Artificial Sequence: Synthetic primer
  source 1..24: mol_type = other DNA
gaacaacctgaaatatctatttcc

SEQ ID NO: 16 - DNA, 19 bp, synthetic construct
  misc_feature 1..19: note = Description of Artificial Sequence: Synthetic primer
  source 1..19: mol_type = other DNA
catatcttggaaggcatcc

SEQ ID NO: 17 - DNA, 20 bp, synthetic construct
  misc_feature 1..20: note = Description of Artificial Sequence: Synthetic primer
  source 1..20: mol_type = other DNA
gggtcatctgctgaaatcac

SEQ ID NO: 18 - DNA, 15 bp, synthetic construct
  misc_feature 1..15: note = Description of Artificial Sequence: Synthetic primer
  source 1..15: mol_type = other DNA
ccaggaggggaaggc

SEQ ID NO: 19 - DNA, 16 bp, synthetic construct
  misc_feature 1..16: note = Description of Artificial Sequence: Synthetic primer
  source 1..16: mol_type = other DNA
ggtgttcccctcctgg

SEQ ID NO: 20 - DNA, 17 bp, synthetic construct
  misc_feature 1..17: note = Description of Artificial Sequence: Synthetic primer
  source 1..17: mol_type = other DNA
cccagaatcgtgttgct

SEQ ID NO: 21 - DNA, 19 bp, synthetic construct
  misc_feature 1..19: note = Description of Artificial Sequence: Synthetic primer
  source 1..19: mol_type = other DNA
gcccagaaggttactcaag

SEQ ID NO: 22 - DNA, 22 bp, synthetic construct
  misc_feature 1..22: note = Description of Artificial Sequence: Synthetic primer
  source 1..22: mol_type = other DNA
agcaaagagatgattttcctta

SEQ ID NO: 23 - DNA, 19 bp, synthetic construct
  misc_feature 1..19: note = Description of Artificial Sequence: Synthetic primer
  source 1..19: mol_type = other DNA
attgagttggtgcctgaac

SEQ ID NO: 24 - DNA, 24 bp, synthetic construct
  misc_feature 1..24: note = Description of Artificial Sequence: Synthetic primer
  source 1..24: mol_type = other DNA
tatatcaactggtacaggaagacc

SEQ ID NO: 25 - DNA, 23 bp, synthetic construct
  misc_feature 1..23: note = Description of Artificial Sequence: Synthetic primer
  source 1..23: mol_type = other DNA
tgtgacaaagtaacccagagttc

SEQ ID NO: 26 - DNA, 22 bp, synthetic construct
  misc_feature 1..22: note = Description of Artificial Sequence: Synthetic primer
  source 1..22: mol_type = other DNA
ggtactgctctgcacttacgac

SEQ ID NO: 27 - DNA, 18 bp, synthetic construct
  misc_feature 1..18: note = Description of Artificial Sequence: Synthetic primer
  source 1..18: mol_type = other DNA
caaacccaaccaggaatg

SEQ ID NO: 28 - DNA, 19 bp, synthetic construct
  misc_feature 1..19: note = Description of Artificial Sequence: Synthetic primer
  source 1..19: mol_type = other DNA
aggaaaaggaggctgtgac

SEQ ID NO: 29 - DNA, 21 bp, synthetic construct
  misc_feature 1..21: note = Description of Artificial Sequence: Synthetic primer
  source 1..21: mol_type = other DNA
gcaagttaagcaaaattcacc

SEQ ID NO: 30 - DNA, 20 bp, synthetic construct
  misc_feature 1..20: note = Description of Artificial Sequence: Synthetic primer
  source 1..20: mol_type = other DNA
ctgctgaaggtcctacattc

SEQ ID NO: 31 - DNA, 20 bp, synthetic construct
  misc_feature 1..20: note = Description of Artificial Sequence: Synthetic primer
  source 1..20: mol_type = other DNA
ttgatagtccagaaaggagg

SEQ ID NO: 32 - DNA, 20 bp, synthetic construct
  misc_feature 1..20: note = Description of Artificial Sequence: Synthetic primer
  source 1..20: mol_type = other DNA
cgtttgactactttccatgg

SEQ ID NO: 33 - DNA, 19 bp, synthetic construct
  misc_feature 1..19: note = Description of Artificial Sequence: Synthetic primer
  source 1..19: mol_type = other DNA
gacaaggtggtacaaagcc

SEQ ID NO: 34 - DNA, 19 bp, synthetic construct
  misc_feature 1..19: note = Description of Artificial Sequence: Synthetic primer
  source 1..19: mol_type = other DNA
atctctggttgtccacgag

SEQ ID NO: 35 - DNA, 21 bp, synthetic construct
  misc_feature 1..21: note = Description of Artificial Sequence: Synthetic primer
  source 1..21: mol_type = other DNA
cagtcactcagtctcaaccag

SEQ ID NO: 36 - DNA, 18 bp, synthetic construct
  misc_feature 1..18: note = Description of Artificial Sequence: Synthetic primer
  source 1..18: mol_type = other DNA
tctggtacaagcagcctc

SEQ ID NO: 37 - DNA, 24 bp, synthetic construct
  misc_feature 1..24: note = Description of Artificial Sequence: Synthetic primer
  source 1..24: mol_type = other DNA
cttcatatttaccaagcttgacag

SEQ ID NO: 38 - DNA, 22 bp, synthetic construct
  misc_feature 1..22: note = Description of Artificial Sequence: Synthetic primer
  source 1..22: mol_type = other DNA
gatgacaatagcaggatcaaac

SEQ ID NO: 39 - DNA, 51 bp, synthetic construct
  misc_feature 1..51: note = Description of Artificial Sequence: Synthetic primer
  source 1..51: mol_type = other DNA
ccctcactccttctctaggcgccggaattcgccaggatggaacatagcacg

SEQ ID NO: 40 - DNA, 68 bp, synthetic construct
  misc_feature 1..68: note = Description of Artificial Sequence: Synthetic primer
  source 1..68: mol_type = other DNA
ccacgtctcccgccaacttgagaaggtcaaaattcaaagtctgtttcaccggtcccttgttccgagcc

SEQ ID NO: 41 - DNA, 67 bp, synthetic construct
  misc_feature 1..67: note = Description of Artificial Sequence: Synthetic primer
  source 1..67: mol_type = other DNA
gaattttgaccttctcaagttggcgggagacgtggagtccaacccagggcccatggaacaggggaag

SEQ ID NO: 42 - DNA, 62 bp, synthetic construct
  misc_feature 1..62: note = Description of Artificial Sequence: Synthetic primer
  source 1..62: mol_type = other DNA
cctcgacgtcaccgcatgttagcagacttcctctgccctcagatcttctattcctcctcaac

SEQ ID NO: 43 - DNA, 65 bp, synthetic construct
  misc_feature 1..65: note = Description of Artificial Sequence: Synthetic primer
  source 1..65: mol_type = other DNA
cagaggaagtctgctaacatgcggtgacgtcgaggagaatcctggcccaatgcagtcgggcactc

SEQ ID NO: 44 - DNA, 69 bp, synthetic construct
  misc_feature 1..69: note = Description of Artificial Sequence: Synthetic primer
  source 1..69: mol_type = other DNA
gttttcttccacgtctcctgcttgctttaacagagagaagttcgtggcggatcctccgatgcgtctctg

SEQ ID NO: 45 - DNA, 61 bp, synthetic construct
  misc_feature 1..61: note = Description of Artificial Sequence: Synthetic primer
  source 1..61: mol_type = other DNA
ctctctgttaaagcaagcaggagacgtggaagaaaaccccggtcccatgaagtggaaagtg

SEQ ID NO: 46 - DNA, 50 bp, synthetic construct
  misc_feature 1..50: note = Description of Artificial Sequence: Synthetic primer
  source 1..50: mol_type = other DNA
gagggagaggggcggaattgatcctcgagcaattgttagcgaggggccag

SEQ ID NO: 47 - protein, 22 aa, Foot-and-mouth disease virus
VKQTLNFDLLKLAGDVESNPGP

SEQ ID NO: 48 - protein, 18 aa, Thosea asigna virus
EGRGSLLTCGDVEENPGP

SEQ ID NO: 49 - protein, 19 aa, Teschovirus A
  source 1..19: note = Porcine teschovirus 1
ATNFSLLKQAGDVEENPGP

SEQ ID NO: 50 - DNA, 702 bp, synthetic construct
  misc_feature 1..702: note = Description of Artificial Sequence: Synthetic polynucleotide
  source 1..702: mol_type = other DNA
catacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacagatgtcatcacaatggatcccaaagacaattggtcaaaagatgcaaatgatacactactgctgcagctcacaaacacctctgcatattacatgtacctcctcctgctcctcaagagtgtggtctattttgccatcatcacctgctgtctgcttggaagaacggctttctgctgcaatggagagaaatcagccacgaacttctctctgttaaagcaagcaggagacgtggaagaaaaccccggtcccatgctgttctccagcctgctgtgtgtatttgtggccttcagctactctggatcaagtgtggcccagaaggttactcaagcccagtcatcagtatccatgccagtgaggaaagcagtcaccctgaactgcctgtatgaaacaagttggtggtcatattatattttttggtacaagcaacttcccagcaaagagatgattttccttattcgcc

SEQ ID NO: 51 - DNA, 676 bp, synthetic construct
  misc_feature 1..676: note = Description of Artificial Sequence: Synthetic polynucleotide
  source 1..676: mol_type = other DNA
catacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacagatgtcatcacaatggatcccaaagacaattggtcaaaagatgcaaatgatacactactgctgcagctcacaaacacctctgcatattacatgtacctcctcctgctcctcaagagtgtggtctattttgccatcatcacctgctgtctgcttggaagaacggctttctgctgcaatggagagaaatcagccacgaacttctctctgttaaagcaagcaggagacgtggaagaaaaccccggtcccatgcagaggatctcctccctcatccatctctctctcttctgggcaggagtcatgtcagccattgagttggtgcctgaacaccaaacagtgcctgtgtcaataggggtccctgccaccctcaggtgctccatgaaaggagaagcgatcggtaactactatatcaactggtacaggaagacccaagg

SEQ ID NO: 52 - DNA, 624 bp, synthetic construct
  misc_feature 1..624: note = Description of Artificial Sequence: Synthetic polynucleotide
  source 1..624: mol_type = other DNA
catacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacagatgtcatcacaatggatcccaaagacaattggtcaaaagatgcaaatgatacactactgctgcagctcacaaacacctctgcatattacatgtacctcctcctgctcctcaagagtgtggtctattttgccatcatcacctgctgtctgcttggaagaacggctttctgctgcaatggagagaaatcagccacgaacttctctctgttaaagcaagcaggagacgtggaagaaaaccccggtcccatgattcttactgtgggctttagctttttgtttttctacaggggcacgctgtgtgacaaagtaacccagagttccccggaccagacggtggcgagtggcagtgaggtggtactgctctgcacttacgacactg

SEQ ID NO: 53 - DNA, 615 bp, synthetic construct
  misc_feature 1..615: note = Description of Artificial Sequence: Synthetic polynucleotide
  source 1..615: mol_type = other DNA
catacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacagatgtcatcacaatggatcccaaagacaattggtcaaaagatgcaaatgatacactactgctgcagctcacaaacacctctgcatattacatgtacctcctcctgctcctcaagagtgtggtctattttgccatcatcacctgctgtctgcttggaagaacggctttctgctgcaatggagagaaatcagccacgaacttctctctgttaaagcaagcaggagacgtggaagaaaaccccggtcccatgtcactttctagcctgctgaaggtggtcacagcttcactgtggctaggacctggcattgcccagaagataactcaaacccaaccaggaatgttcgtgcaggaaaaggaggctgtgactctgg

SEQ ID NO: 54 - DNA, 715 bp, synthetic construct
  misc_feature 1..715: note = Description of Artificial Sequence: Synthetic polynucleotide
  source 1..715: mol_type = other DNA
catacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacagatgtcatcacaatggatcccaaagacaattggtcaaaagatgcaaatgatacactactgctgcagctcacaaacacctctgcatattacatgtacctcctcctgctcctcaagagtgtggtctattttgccatcatcacctgctgtctgcttggaagaacggctttctgctgcaatggagagaaatcagccacgaacttctctctgttaaagcaagcaggagacgtggaagaaaaccccggtcccatggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatc

SEQ ID NO: 55 - DNA, 684 bp, synthetic construct
  misc_feature 1..684: note = Description of Artificial Sequence: Synthetic polynucleotide
  source 1..684: mol_type = other DNA
catacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacagatgtcatcacaatggatcccaaagacaattggtcaaaagatgcaaatgatacactactgctgcagctcacaaacacctctgcatattacatgtacctcctcctgctcctcaagagtgtggtctattttgccatcatcacctgctgtctgcttggaagaacggctttctgctgcaatggagagaaatcagccacgaacttctctctgttaaagcaagcaggagacgtggaagaaaaccccggtcccatggacaagatcttaggagcatcatttttagttctgtggcttcaactatgctgggtgagtggccaacagaaggagaaaagtgaccagcagcaggtgaaacaaagtcctcaatctttgatagtccagaaaggagggatttcaattataaactgtgcttatgagaacactgcgtttgactactttccatggtacc

SEQ ID NO: 56 - DNA, 603 bp, synthetic construct
  misc_feature 1..603: note = Description of Artificial Sequence: Synthetic polynucleotide
  source 1..603: mol_type = other DNA
catacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacagatgtcatcacaatggatcccaaagacaattggtcaaaagatgcaaatgatacactactgctgcagctcacaaacacctctgcatattacatgtacctcctcctgctcctcaagagtgtggtctattttgccatcatcacctgctgtctgcttggaagaacggctttctgctgcaatggagagaaatcagccacgaacttctctctgttaaagcaagcaggagacgtggaagaaaaccccggtcccatgatgaagtgtccacaggctttactagctatcttttggcttctactgagctgggtgagcagtgaagacaaggtggtacaaagccctctatctctggttgtccacgagggag

SEQ ID NO: 57 - DNA, 676 bp, synthetic construct
  misc_feature 1..676: note = Description of Artificial Sequence: Synthetic polynucleotide
  source 1..676: mol_type = other DNA
catacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacagatgtcatcacaatggatcccaaagacaattggtcaaaagatgcaaatgatacactactgctgcagctcacaaacacctctgcatattacatgtacctcctcctgctcctcaagagtgtggtctattttgccatcatcacctgctgtctgcttggaagaacggctttctgctgcaatggagagaaatcagccacgaacttctctctgttaaagcaagcaggagacgtggaagaaaaccccggtcccatggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccag

SEQ ID NO: 58 - protein, 13 aa, Homo sapiens
ALFIQELGKKIKV

SEQ ID NO: 59 - protein, 13 aa, Homo sapiens
ACDVLGDTEGRLI

SEQ ID NO: 60 - protein, 11 aa, Homo sapiens
ALWDGPYYKKL

SEQ ID NO: 61 - protein, 19 aa, Homo sapiens
ACDTVFTGGYSSWDTRQMF

SEQ ID NO: 62 - protein, 17 aa, Homo sapiens
ALWDIPPGQELGKKIKV

SEQ ID NO: 63 - protein, 17 aa, Homo sapiens
ACDTLGETSSWDTRQMF

SEQ ID NO: 64 - protein, 14 aa, Homo sapiens
ALWEAQELGKKIKV

SEQ ID NO: 65 - protein, 16 aa, Homo sapiens
ACDSGGYSSWDTRQMF

SEQ ID NO: 66 - protein, 15 aa, Homo sapiens
ALWEARQELGKKIKV

SEQ ID NO: 67 - protein, 16 aa, Homo sapiens
ACDTLFPGGSATDKLI

SEQ ID NO: 68 - protein, 17 aa, Homo sapiens
ALWEGTRGQELGKKIKV

SEQ ID NO: 69 - protein, 13 aa, Homo sapiens
ACDTVGAHTDKLI

SEQ ID NO: 70 - protein, 16 aa, Homo sapiens
ALWEVGDQELGKKIKV

SEQ ID NO: 71 - protein, 19 aa, Homo sapiens
ACDPLNTGGSFSLYTDKLI

SEQ ID NO: 72 - protein, 15 aa, Homo sapiens
ALWEVHSELGKKIKV

SEQ ID NO: 73 - protein, 17 aa, Homo sapiens
ACDTGGFRSSWDTRQMF

SEQ ID NO: 74 - protein, 14 aa, Homo sapiens
ALWEVLELGKKIKV

SEQ ID NO: 75 - protein, 16 aa, Homo sapiens
ACDTVGMGIRLGDKLI

SEQ ID NO: 76 - protein, 16 aa, Homo sapiens
ALWEVLVGELGKKIKV

SEQ ID NO: 77 - protein, 13 aa, Homo sapiens
ACDILGINTDKLI

SEQ ID NO: 78 - protein, 14 aa, Homo sapiens
ALWEVPELGKKIKV

SEQ ID NO: 79 - protein, 14 aa, Homo sapiens
ACERLGDYVPDKLI

SEQ ID NO: 80 - protein, 14 aa, Homo sapiens
ALWEVQELGKKIKV

SEQ ID NO: 81 - protein, 13 aa, Homo sapiens
ACDRLLGDTDKLI

SEQ ID NO: 82 - protein, 19 aa, Homo sapiens
ACDTVAPRIGGLKYTDKLI

SEQ ID NO: 83 - protein, 14 aa, Homo sapiens
ACDTVGGPYTDKLI

SEQ ID NO: 84 - protein, 10 aa, Homo sapiens
ACDTVGGTAQ

SEQ ID NO: 85 - protein, 19 aa, Homo sapiens
ACDTVSGGSTPTWYTDKLI

SEQ ID NO: 86 - protein, 17 aa, Homo sapiens
ACDTVSIFTGDTTDKLI

SEQ ID NO: 87 - protein, 14 aa, Homo sapiens
ALWEVRELGKKIKV

SEQ ID NO: 88 - protein, 19 aa, Homo sapiens
ACDTILIFSPTGGDTDKLI

SEQ ID NO: 89 - protein, 13 aa, Homo sapiens
ACVPLGDWTDKLI

SEQ ID NO: 90 - protein, 16 aa, Homo sapiens
ALWEVRKQELGKKIKV

SEQ ID NO: 91 - protein, 13 aa, Homo sapiens
ACDTLGDDFDKLI

SEQ ID NO: 92 - protein, 18 aa, Homo sapiens
ALWEVTHNRQELGKKIKV

SEQ ID NO: 93 - protein, 17 aa, Homo sapiens
ACDTLLGTEAWDTRQMF

SEQ ID NO: 94 - protein, 14 aa, Homo sapiens
ALWGGAAGAYYKKL

SEQ ID NO: 95 - protein, 13 aa, Homo sapiens
ACDGKTTDTDKLI

SEQ ID NO: 96 - protein, 13 aa, Homo sapiens
ALWGGELGKKIKV

SEQ ID NO: 97 - protein, 15 aa, Homo sapiens
ACDLLGDTRYTDKLI

SEQ ID NO: 98 - protein, 13 aa, Homo sapiens
ALWVQELGKKIKV

SEQ ID NO: 99 - protein, 13 aa, Homo sapiens
ACVGITGDTDKLI

SEQ ID NO: 100 - protein, 15 aa, Homo sapiens
ALWEAHQELGKKIKV

SEQ ID NO: 101 - protein, 13 aa, Homo sapiens
ACDSLGDSVDKLI

SEQ ID NO: 102 - protein, 9 aa, Homo sapiens
ALWEANKKL

SEQ ID NO: 103 - protein, 16 aa, Homo sapiens
ACDLLRGAGGQIDKLI

SEQ ID NO: 104 - protein, 14 aa, Homo sapiens
ALWEAQELGKKIKV

SEQ ID NO: 105 - protein, 15 aa, Homo sapiens
ACDTVGGAFDTDKLI

SEQ ID NO: 106 - protein, 14 aa, Homo sapiens
ALWEATGLGKKIKV

SEQ ID NO: 107 - protein, 16 aa, Homo sapiens
ACDMGDTRSWDTRQMF

SEQ ID NO: 108 - protein, 14 aa, Homo sapiens
ALWEDLELGKKIKV

SEQ ID NO: 109 - protein, 15 aa, Homo sapiens
ACDTVSWGKNTDKLI

SEQ ID NO: 110 - protein, 14 aa, Homo sapiens
ALWEKEELGKKIKV

SEQ ID NO: 111 - protein, 17 aa, Homo sapiens
ACDTGDWGSSWDTRQMF

SEQ ID NO: 112 - protein, 13 aa, Homo sapiens
ALWEKELGKKIKV

SEQ ID NO: 113 - protein, 18 aa, Homo sapiens
ACDILDSTGGTDLTAQLF

SEQ ID NO: 114 - protein, 15 aa, Homo sapiens
ALWEMTQELGKKIKV

SEQ ID NO: 115 - protein, 20 aa, Homo sapiens
ACDTVRNTGGYAFAGIDKLI

SEQ ID NO: 116 - protein, 14 aa, Homo sapiens
ALWEPQELGKKIKV

SEQ ID NO: 117 - protein, 17 aa, Homo sapiens
ACDKVLGDSSWDTRQMF

SEQ ID NO: 118 - protein, 14 aa, Homo sapiens
ALWESKELGKKIKV

SEQ ID NO: 119 - protein, 18 aa, Homo sapiens
ACEGLGATQSSWDTRQMF

SEQ ID NO: 120 - protein, 14 aa, Homo sapiens
ALWEVGELGKKIKV

SEQ ID NO: 121 - protein, 12 aa, Homo sapiens
ACDKLLGDNELI

SEQ ID NO: 122 - protein, 14 aa, Homo sapiens
ALWEVHKLGKKIKV

SEQ ID NO: 123 - protein, 14 aa, Homo sapiens
ACDSLLGKGTDKLI

SEQ ID NO: 124 - protein, 14 aa, Homo sapiens
ALWEVKELGKKIKV

SEQ ID NO: 125 - protein, 13 aa, Homo sapiens
ACDTLRGSADKLI

SEQ ID NO: 126 - protein, 16 aa, Homo sapiens
ALWEVLQQELGKKIKV

SEQ ID NO: 127 - protein, 14 aa, Homo sapiens
ACDTVPARHTDKLI

SEQ ID NO: 128 - protein, 14 aa, Homo sapiens
ALWEVPVLGKKIKV

SEQ ID NO: 129 - protein, 15 aa, Homo sapiens
ACDTADRSSYTDKLI

SEQ ID NO: 130 - protein, 19 aa, Homo sapiens
ACDTLLGDPSSSWDTRQMF

SEQ ID NO: 131 - protein, 16 aa, Homo sapiens
ACDTLSGGYARTDKLI

SEQ ID NO: 132 - protein, 16 aa, Homo sapiens
ACDTVGILGDTGLGLI

SEQ ID NO: 133 - protein, 16 aa, Homo sapiens
ACDTIVSGYDGYDKLI

SEQ ID NO: 134 - protein, 14 aa, Homo sapiens
ACSILGDKTSDKLI

SEQ ID NO: 135 - protein, 15 aa, Homo sapiens
ALWEVRQELGKKIKV

SEQ ID NO: 136 - protein, 16 aa, Homo sapiens
ACDTVSQRGGYSDKLI

SEQ ID NO: 137 - protein, 16 aa, Homo sapiens
ALWEVRVQELGKKIKV

SEQ ID NO: 138 - protein, 18 aa, Homo sapiens
ACDPLERVGGPANTDKLI

SEQ ID NO: 139 - protein, 14 aa, Homo sapiens
ALWEVTELGKKIKV

SEQ ID NO: 140 - protein, 14 aa, Homo sapiens
ACDVLGDTGDDKLI

SEQ ID NO: 141 - protein, 13 aa, Homo sapiens
ALWGRELGKKIKV

SEQ ID NO: 142 - protein, 13 aa, Homo sapiens
ACDTVGSNTDKLI

SEQ ID NO: 143 - protein, 14 aa, Homo sapiens
ACDVLGDTEADKLI

SEQ ID NO: 144 - protein, 17 aa, Homo sapiens
ALYGSPSGEELGKKNQG

SEQ ID NO: 145 - protein, 17 aa, Homo sapiens
ACDPLEGAGGHNTDKLI

SEQ ID NO: 146 - protein, 9 aa, Influenza virus
GILGFVFTL

SEQ ID NO: 147 - DNA, 17 bp, synthetic construct
  misc_feature 1..17: note = Description of Artificial Sequence: Synthetic primer
  source 1..17: mol_type = other DNA
gcagctggagcaaactg

SEQ ID NO: 148 - DNA, 19 bp, synthetic construct
  misc_feature 1..19: note = Description of Artificial Sequence: Synthetic primer
  source 1..19: mol_type = other DNA
ctgaattatcggtcaccag

SEQ ID NO: 149 - DNA, 24 bp, synthetic construct
  misc_feature 1..24: note = Description of Artificial Sequence: Synthetic primer
  source 1..24: mol_type = other DNA
caaatatcctgtaaagttttcatc

SEQ ID NO: 150 - DNA, 31 bp, synthetic construct
  misc_feature 1..31: note = Description of Artificial Sequence: Synthetic primer
  source 1..31: mol_type = other DNA
gtttagagtttctattatatgtccttgcaac

SEQ ID NO: 151 - DNA, 22 bp, synthetic construct
  misc_feature 1..22: note = Description of Artificial Sequence: Synthetic primer
  source 1..22: mol_type = other DNA
gatatctcaggatcagctctcc

SEQ ID NO: 152 - DNA, 21 bp, synthetic construct
  misc_feature 1..21: note = Description of Artificial Sequence: Synthetic primer
  source 1..21: mol_type = other DNA
tacccgaagaccaaacaagac

SEQ ID NO: 153 - DNA, 19 bp, synthetic construct
  misc_feature 1..19: note = Description of Artificial Sequence: Synthetic primer
  source 1..19: mol_type = other DNA
tcacctctggggtcatatg

SEQ ID NO: 154 - DNA, 19 bp, synthetic construct
  misc_feature 1..19: note = Description of Artificial Sequence: Synthetic primer
  source 1..19: mol_type = other DNA
agaggaaaggaaatacggc

SEQ ID NO: 155 - DNA, 24 bp, synthetic construct
  misc_feature 1..24: note = Description of Artificial Sequence: Synthetic primer
  source 1..24: mol_type = other DNA
caacttggaagaaagaataatgtc

SEQ ID NO: 156 - DNA, 18 bp, synthetic construct
  misc_feature 1..18: note = Description of Artificial Sequence: Synthetic primer
  source 1..18: mol_type = other DNA
caccaagctagaggggtc

SEQ ID NO: 157 - DNA, 20 bp, synthetic construct
  misc_feature 1..20: note = Description of Artificial Sequence: Synthetic primer
  source 1..20: mol_type = other DNA
cttttctttccaatacaccc

SEQ ID NO: 158 - DNA, 21 bp, synthetic construct
  misc_feature 1..21: note = Description of Artificial Sequence: Synthetic primer
  source 1..21: mol_type = other DNA
tcdggaaagaacttttcaagg

SEQ ID NO: 159 - DNA, 18 bp, synthetic construct
  misc_feature 1..18: note = Description of Artificial Sequence: Synthetic primer
  source 1..18: mol_type = other DNA
acccaaatgttgcatcag

SEQ ID NO: 160 - DNA, 21 bp, synthetic construct
  misc_feature 1..21: note = Description of Artificial Sequence: Synthetic primer
  source 1..21: mol_type = other DNA
gtctctgacaatccaagaagg

SEQ ID NO: 161 - DNA, 16 bp, synthetic construct
  misc_feature 1..16: note = Description of Artificial Sequence: Synthetic primer
  source 1..16: mol_type = other DNA
tctgtgcaggtggcag

SEQ ID NO: 162 - DNA, 18 bp, synthetic construct
  misc_feature 1..18: note = Description of Artificial Sequence: Synthetic primer
  source 1..18: mol_type = other DNA
ccctggactgcacctatg

SEQ ID NO: 163 - DNA, 21 bp, synthetic construct
  misc_feature 1..21: note = Description of Artificial Sequence: Synthetic primer
  source 1..21: mol_type = other DNA
tgtatatttggaaccagttgc

SEQ ID NO: 164 - DNA, 20 bp, synthetic construct
  misc_feature 1..20: note = Description of Artificial Sequence: Synthetic primer
  source 1..20: mol_type = other DNA
gatcctgcctccttctactg

SEQ ID NO: 165 - DNA, 16 bp, synthetic construct
  misc_feature 1..16: note = Description of Artificial Sequence: Synthetic primer
  source 1..16: mol_type = other DNA
catcacgctgacccag

SEQ ID NO: 166 - DNA, 18 bp, synthetic construct
  misc_feature 1..18: note = Description of Artificial Sequence: Synthetic primer
  source 1..18: mol_type = other DNA
gctccactgaccagacag

SEQ ID NO: 167 - DNA, 22 bp, synthetic construct
  misc_feature 1..22: note = Description of Artificial Sequence: Synthetic primer
  source 1..22: mol_type = other DNA
cascttyttagtggagagatgg

SEQ ID NO: 168 - DNA, 24 bp, synthetic construct
  misc_feature 1..24: note = Description of Artificial Sequence: Synthetic primer
  source 1..24: mol_type = other DNA
aytctgtagtcttccagaaatcac

SEQ ID NO: 169 - DNA, 16 bp, synthetic construct
  misc_feature 1..16: note = Description of Artificial Sequence: Synthetic primer
  source 1..16: mol_type = other DNA
tccttggttctgcagg

SEQ ID NO: 170 - DNA, 15 bp, synthetic construct
  misc_feature 1..15: note = Description of Artificial Sequence: Synthetic primer
  source 1..15: mol_type = other DNA
tgcaggagggggaga

SEQ ID NO: 171 - DNA, 18 bp, synthetic construct
  misc_feature 1..18: note = Description of Artificial Sequence: Synthetic primer
  source 1..18: mol_type = other DNA
gcagcaggtgagacaaag

SEQ ID NO: 172 - DNA, 19 bp, synthetic construct
  misc_feature 1..19: note = Description of Artificial Sequence: Synthetic primer
  source 1..19: mol_type = other DNA
ctctgacagtctgggaagg

SEQ ID NO: 173 - DNA, 20 bp, synthetic construct
  misc_feature 1..20: note = Description of Artificial Sequence: Synthetic primer
  source 1..20: mol_type = other DNA
cagatgcaaggtcaagtgac

SEQ ID NO: 174 - DNA, 18 bp, synthetic construct
  misc_feature 1..18: note = Description of Artificial Sequence: Synthetic primer
  source 1..18: mol_type = other DNA
ggagaaggtccacagctc

SEQ ID NO: 175 - DNA, 18 bp, synthetic construct
  misc_feature 1..18: note = Description of Artificial Sequence: Synthetic primer
  source 1..18: mol_type = other DNA
aaggtccacagctccttc

SEQ ID NO: 176 - DNA, 18 bp, synthetic construct
  misc_feature 1..18: note = Description of Artificial Sequence: Synthetic primer
  source 1..18: mol_type = other DNA
caactgccaacaacaagg

SEQ ID NO: 177 - DNA, 21 bp, synthetic construct
  misc_feature 1..21: note = Description of Artificial Sequence: Synthetic primer
  source 1..21: mol_type = other DNA
gttctggtatgtgcagtatcc

SEQ ID NO: 178 - DNA, 19 bp, synthetic construct
  misc_feature 1..19: note = Description of Artificial Sequence: Synthetic primer
  source 1..19: mol_type = other DNA
tccttccacttgcagaaag

SEQ ID NO: 179 - DNA, 21 bp, synthetic construct
  misc_feature 1..21: note = Description of Artificial Sequence: Synthetic primer
  source 1..21: mol_type = other DNA
tctgstctgagatgcaatttt

SEQ ID NO: 180 - DNA, 22 bp, synthetic construct
  misc_feature 1..22: note = Description of Artificial Sequence: Synthetic primer
  modified_base 3: mod_base = OTHER; note = Inosine
  modified_base 5: mod_base = OTHER; note = Inosine
  source 1..22: mol_type = other DNA
ggntnmaggaacaaaggagaat

SEQ ID NO: 181 - DNA, 20 bp, synthetic construct
  misc_feature 1..20: note = Description of Artificial Sequence: Synthetic primer
  source 1..20: mol_type = other DNA
gtacaagcaaacagcaagtg

SEQ ID NO: 182 - DNA, 24 bp, synthetic construct
  misc_feature 1..24: note = Description of Artificial Sequence: Synthetic primer
  source 1..24: mol_type = other DNA
attattctctgaactttcagaagc

SEQ ID NO: 183 - DNA, 18 bp, synthetic construct
  misc_feature 1..18: note = Description of Artificial Sequence: Synthetic primer
  source 1..18: mol_type = other DNA
gtgcacttgccttgtagc

SEQ ID NO: 184 - DNA, 20 bp, synthetic construct
  misc_feature 1..20: note = Description of Artificial Sequence: Synthetic primer
  source 1..20: mol_type = other DNA
aatagtatggctttcctggc

SEQ ID NO: 185 - DNA, 23 bp, synthetic construct
  misc_feature 1..23: note = Description of Artificial Sequence: Synthetic primer
  source 1..23: mol_type = other DNA
tgaaagaattttgcatatggttc

SEQ ID NO: 186 - DNA, 21 bp, synthetic construct
  misc_feature 1..21: note = Description of Artificial Sequence: Synthetic primer
  source 1..21: mol_type = other DNA
gagatgactatagcagggtcg

SEQ ID NO: 187 - DNA, 18 bp, synthetic construct
  misc_feature 1..18: note = Description of Artificial Sequence: Synthetic primer
  source 1..18: mol_type = other DNA
taccacgtggtcaagctg

SEQ ID NO: 188 - DNA, 17 bp, synthetic construct
  misc_feature 1..17: note = Description of Artificial Sequence: Synthetic primer
  source 1..17: mol_type = other DNA
ggggttgtccagtctcc

SEQ ID NO: 189 - DNA, 17 bp, synthetic construct
  misc_feature 1..17: note = Description of Artificial Sequence: Synthetic primer
  source 1..17: mol_type = other DNA
gctgcagtcacccaaag

SEQ ID NO: 190 - DNA, 19 bp, synthetic construct
  misc_feature 1..19: note = Description of Artificial Sequence: Synthetic primer
  source 1..19: mol_type = other DNA
cctaggcacaaggtgacag

SEQ ID NO: 191 - DNA, 18 bp, synthetic construct
  misc_feature 1..18: note = Description of Artificial Sequence: Synthetic primer
  source 1..18: mol_type = other DNA
gcagtcctacaggaaggg

SEQ ID NO: 192 - DNA, 19 bp, synthetic construct
  misc_feature 1..19: note = Description of Artificial Sequence: Synthetic primer
  source 1..19: mol_type = other DNA
gagttacccagacacccag

SEQ ID NO: 193 - DNA, 17 bp, synthetic construct
  misc_feature 1..17: note = Description of Artificial Sequence: Synthetic primer
  source 1..17: mol_type = other DNA
gaagccaaaccaagcac

SEQ ID NO: 194 - DNA, 17 bp, synthetic construct
  misc_feature 1..17: note = Description of Artificial Sequence: Synthetic primer
  source 1..17: mol_type = other DNA
gattggtcaggaagggc

SEQ ID NO: 195 - DNA, 20 bp, synthetic construct
  misc_feature 1..20: note = Description of Artificial Sequence: Synthetic primer
  source 1..20: mol_type = other DNA
catctactggtaccgacagg

SEQ ID NO: 196 - DNA, 20 bp, synthetic construct
  misc_feature 1..20: note = Description of Artificial Sequence: Synthetic primer
  source 1..20: mol_type = other DNA
cagtatctaggccacaatgc

SEQ ID NO: 197 - DNA, 18 bp, synthetic construct
  misc_feature 1..18: note = Description of Artificial Sequence: Synthetic primer
  source 1..18: mol_type = other DNA
ggatggagtgtcaagctg

SEQ ID NO: 198 - DNA, 19 bp, synthetic construct
  misc_feature 1..19: note = Description of Artificial Sequence: Synthetic primer
  source 1..19: mol_type = other DNA
ctgcagttacacagaagcc

SEQ ID NO: 199 - DNA, 19 bp, synthetic construct
  misc_feature 1..19: note = Description of Artificial Sequence: Synthetic primer
  source 1..19: mol_type = other DNA
cagactccacgatacctgg

SEQ ID NO: 200 - DNA, 17 bp, synthetic construct
  misc_feature 1..17: note = Description of Artificial Sequence: Synthetic primer
  source 1..17: mol_type = other DNA
gctggaatgtggacagg

SEQ ID NO: 201 - DNA, 20 bp, synthetic construct
  misc_feature 1..20: note = Description of Artificial Sequence: Synthetic primer
  source 1..20: mol_type = other DNA
cccaaagtcttacagatccc

SEQ ID NO: 202 - DNA, 23 bp, synthetic construct
  misc_feature 1..23: note = Description of Artificial Sequence: Synthetic primer
  source 1..23: mol_type = other DNA
ctaacctctactggtactggcag

SEQ ID NO: 203 - DNA, 18 bp, synthetic construct
  misc_feature 1..18: note = Description of Artificial Sequence: Synthetic primer
  source 1..18: mol_type = other DNA
gacggctgttttccagac

SEQ ID NO: 204 - DNA, 20 bp, synthetic construct
  misc_feature 1..20: note = Description of Artificial Sequence: Synthetic primer
  source 1..20: mol_type = other DNA
ggtataaacagagcgctgag

SEQ ID NO: 205 - DNA, 20 bp, synthetic construct
  misc_feature 1..20: note = Description of Artificial Sequence: Synthetic primer
  source 1..20: mol_type = other DNA
ccagaaggtagcagagaccc

SEQ ID NO: 206 - DNA, 18 bp, synthetic construct
  misc_feature 1..18: note = Description of Artificial Sequence: Synthetic primer
  source 1..18: mol_type = other DNA
gtatccctggatgagctg

SEQ ID NO: 207 - DNA, 18 bp, synthetic construct
  misc_feature 1..18: note = Description of Artificial Sequence: Synthetic primer
  source 1..18: mol_type = other DNA
ccagcagattctcagtcc

SEQ ID NO: 208 - DNA, 19 bp, synthetic construct
  misc_feature 1..19: note = Description of Artificial Sequence: Synthetic primer
  source 1..19: mol_type = other DNA
gtactggtatcggcaggac

SEQ ID NO: 209 - DNA, 18 bp, synthetic construct
  misc_feature 1..18: note = Description of Artificial Sequence: Synthetic primer
  source 1..18: mol_type = other DNA
ggtatcagcagcccagag

SEQ ID NO: 210 - DNA, 18 bp, synthetic construct
  misc_feature 1..18: note = Description of Artificial Sequence: Synthetic primer
  source 1..18: mol_type = other DNA
gtgtgagccagtttcagg

SEQ ID NO: 211 - DNA, 19 bp, synthetic construct
  misc_feature 1..19: note = Description of Artificial Sequence: Synthetic primer
  source 1..19: mol_type = other DNA
gaagcaactctgtggtgtg

SEQ ID NO: 212 - DNA, 18 bp, synthetic construct
  misc_feature 1..18: note = Description of Artificial Sequence: Synthetic primer
  source 1..18: mol_type = other DNA
gaacagggaagctgacac

SEQ ID NO: 213 - DNA, 18 bp, synthetic construct
  misc_feature 1..18: note = Description of Artificial Sequence: Synthetic primer
  source 1..18: mol_type = other DNA
ggtaccgacaggattcag

SEQ ID NO: 214 - DNA, 18 bp, synthetic construct
  misc_feature 1..18: note = Description of Artificial Sequence: Synthetic primer
  source 1..18: mol_type = other DNA
ggacaatcagactgcctc

SEQ ID NO: 215 - DNA, 18 bp, synthetic construct
  misc_feature 1..18: note = Description of Artificial Sequence: Synthetic primer
  source 1..18: mol_type = other DNA
gcttggtatcgtcaatcg

SEQ ID NO: 216 - DNA, 17 bp, synthetic construct
  misc_feature 1..17: note = Description of Artificial Sequence: Synthetic primer
  source 1..17: mol_type = other DNA
gccaggaagcagagatg

SEQ ID NO: 217 - DNA, 20 bp, synthetic construct
  misc_feature 1..20: note = Description of Artificial Sequence: Synthetic primer
  source 1..20: mol_type = other DNA
gaggtgtatccctgaaaagg

SEQ ID NO: 218 - DNA, 19 bp, synthetic construct
  misc_feature 1..19: note = Description of Artificial Sequence: Synthetic primer
  source 1..19: mol_type = other DNA
gcacactgccttttactgg

SEQ ID NO: 219 - DNA, 21 bp, synthetic construct
  misc_feature 1..21: note = Description of Artificial Sequence: Synthetic primer
  source 1..21: mol_type = other DNA
gtactggtatcgacaagaccc

SEQ ID NO: 220 - DNA, 18 bp, synthetic construct
  misc_feature 1..18: note = Description of Artificial Sequence: Synthetic primer
  source 1..18: mol_type = other DNA
gatatggggcagatggtg

SEQ ID NO: 221 - DNA, 19 bp, synthetic construct
  misc_feature 1..19: note = Description of Artificial Sequence: Synthetic primer
  source 1..19: mol_type = other DNA
ctgttggccaggtagagtc

SEQ ID NO: 222 - DNA, 19 bp, synthetic construct
  misc_feature 1..19: note = Description of Artificial Sequence: Synthetic primer
  source 1..19: mol_type = other DNA
gccagagctcatgtttctc

SEQ ID NO: 223 - DNA, 21 bp, synthetic construct
  misc_feature 1..21: note = Description of Artificial Sequence: Synthetic primer
  source 1..21: mol_type = other DNA
gggtagccttttgtttgtttg

SEQ ID NO: 224 - DNA, 21 bp, synthetic construct
  misc_feature 1..21: note = Description of Artificial Sequence: Synthetic primer
  source 1..21: mol_type = other DNA
ctacactgagtgttcgagagg

SEQ ID NO: 225 - DNA, 15 bp, synthetic construct
  misc_feature 1..15: note = Description of Artificial Sequence: Synthetic primer
  source 1..15: mol_type = other DNA
ggttccacgccactc

SEQ ID NO: 226 - DNA, 18 bp, synthetic construct
  misc_feature 1..18: note = Description of Artificial Sequence: Synthetic primer
  source 1..18: mol_type = other DNA
tatgaaggagcctccctg

SEQ ID NO: 227 - DNA, 17 bp, synthetic construct
  misc_feature 1..17: note = Description of Artificial Sequence: Synthetic primer
  source 1..17: mol_type = other DNA
caagatttcaccgcacg

SEQ ID NO: 228 - DNA, 19 bp, synthetic construct
  misc_feature 1..19: note = Description of Artificial Sequence: Synthetic primer
  source 1..19: mol_type = other DNA
gctgactgttcaagaggga

SEQ ID NO: 229 - DNA, 17 bp, synthetic construct
  misc_feature 1..17: note = Description of Artificial Sequence: Synthetic primer
  source 1..17: mol_type = other DNA
ctccacattcctgagcc

SEQ ID NO: 230 - DNA, 16 bp, synthetic construct
  misc_feature 1..16: note = Description of Artificial Sequence: Synthetic primer
  source 1..16: mol_type = other DNA
actctgagcctgccct

SEQ ID NO: 231 - DNA, 15 bp, synthetic construct
  misc_feature 1..15: note = Description of Artificial Sequence: Synthetic primer
  source 1..15: mol_type = other DNA
acggctggccagaag

SEQ ID NO: 232 - DNA, 17 bp, synthetic construct
  misc_feature 1..17: note = Description of Artificial Sequence: Synthetic primer
  source 1..17: mol_type = other DNA
agagccacccttgacac

SEQ ID NO: 233 - DNA, 19 bp, synthetic construct
  misc_feature 1..19: note = Description of Artificial Sequence: Synthetic primer
  source 1..19: mol_type = other DNA
gcttygaggctgagttcag

SEQ ID NO: 234 - DNA, 19 bp, synthetic construct
  misc_feature 1..19: note = Description of Artificial Sequence: Synthetic primer
  source 1..19: mol_type = other DNA
gcacattgatttgggagtc

SEQ ID NO: 235 - DNA, 19 bp, synthetic construct
  misc_feature 1..19: note = Description of Artificial Sequence: Synthetic primer
  source 1..19: mol_type = other DNA
agagaaggtcgagcaacac

SEQ ID NO: 236 - DNA, 18 bp, synthetic construct
  misc_feature 1..18: note = Description of Artificial Sequence: Synthetic primer
  source 1..18: mol_type = other DNA
aagacccaagtggagcag

SEQ ID NO: 237 - DNA, 17 bp, synthetic construct
  misc_feature 1..17: note = Description of Artificial Sequence: Synthetic primer
  source 1..17: mol_type = other DNA
tgacccagacagaaggc

SEQ ID NO: 238 - DNA, 21 bp, synthetic construct
  misc_feature 1..21: note = Description of Artificial Sequence: Synthetic primer
  source 1..21: mol_type = other DNA
gcaagttaaacaaagctctcc

SEQ ID NO: 239 - DNA, 16 bp, synthetic construct
  misc_feature 1..16: note = Description of Artificial Sequence: Synthetic primer
  source 1..16: mol_type = other DNA
cagtccgtggaccagc

SEQ ID NO: 240 - DNA, 16 bp, synthetic construct
  misc_feature 1..16: note = Description of Artificial Sequence: Synthetic primer
  source 1..16: mol_type = other DNA
aacggctggagcagag

SEQ ID NO: 241 - DNA, 15 bp, synthetic construct
  misc_feature 1..15: note = Description of Artificial Sequence: Synthetic primer
  source 1..15: mol_type = other DNA
ggcgagcaggtggag

SEQ ID NO: 242 - DNA, 26 bp, synthetic construct
  misc_feature 1..26: note = Description of Artificial Sequence: Synthetic primer
  source 1..26: mol_type = other DNA
ggctacttcccttggtataagcaaga

SEQ ID NO: 243 - DNA, 20 bp, synthetic construct
  misc_feature 1..20: note = Description of Artificial Sequence: Synthetic primer
  source 1..20: mol_type = other DNA
agattccgtgactcaaacag

SEQ ID NO: 244 - DNA, 25 bp, synthetic construct
  misc_feature 1..25: note = Description of Artificial Sequence: Synthetic primer
  source 1..25: mol_type = other DNA
agaaggtrcagcagagcccagaatc

SEQ ID NO: 245 - DNA, 17 bp, synthetic construct
  misc_feature 1..17: note = Description of Artificial Sequence: Synthetic primer
  source 1..17: mol_type = other DNA
gagcrtccasgagggtg

SEQ ID NO: 246 - DNA, 18 bp, synthetic construct
  misc_feature 1..18: note = Description of Artificial Sequence: Synthetic primer
  source 1..18: mol_type = other DNA
ccagtggttcaaggagtg

SEQ ID NO: 247 - DNA, 16 bp, synthetic construct
  misc_feature 1..16: note = Description of Artificial Sequence: Synthetic primer
  source 1..16: mol_type = other DNA
ggcatcacagggaacg

SEQ ID NO: 248 - DNA, 20 bp, synthetic construct
  misc_feature 1..20: note = Description of Artificial Sequence: Synthetic primer
  source 1..20: mol_type = other DNA
atctcaccataaactgcacg

SEQ ID NO: 249 - DNA, 21 bp, synthetic construct
  misc_feature 1..21: note = Description of Artificial Sequence: Synthetic primer
  source 1..21: mol_type = other DNA
gcacccacatttctktcttac

SEQ ID NO: 250 - DNA, 22 bp, synthetic construct
  misc_feature 1..22: note = Description of Artificial Sequence: Synthetic primer
  source 1..22: mol_type = other DNA
gaaagaactgcactcttcaatg

SEQ ID NO: 251 - DNA, 21 bp, synthetic construct
  misc_feature 1..21: note = Description of Artificial Sequence: Synthetic primer
  source 1..21: mol_type = other DNA
aagatggaaggtttacagcac

SEQ ID NO: 252 - DNA, 21 bp, synthetic construct
  misc_feature 1..21: note = Description of Artificial Sequence: Synthetic primer
  source 1..21: mol_type = other DNA
tcagacagtgcctcaaactac

SEQ ID NO: 253 - DNA, 21 bp, synthetic construct
  misc_feature 1..21: note = Description of Artificial Sequence: Synthetic primer
  source 1..21: mol_type = other DNA
cagtgaaacatctctctctgc

SEQ ID NO: 254 - DNA, 19 bp, synthetic construct
  misc_feature 1..19: note = Description of Artificial Sequence: Synthetic primer
  source 1..19: mol_type = other DNA
aggctgtgactctggactg

SEQ ID NO: 255 - DNA, 20 bp, synthetic construct
  misc_feature 1..20: note = Description of Artificial Sequence: Synthetic primer
  source 1..20: mol_type = other DNA
gtccagtactccagacaacg

SEQ ID NO: 256 - DNA, 20 bp, synthetic construct
  misc_feature 1..20: note = Description of Artificial Sequence: Synthetic primer
  source 1..20: mol_type = other DNA
ccaccatgaactgcagttac

SEQ ID NO: 257 - DNA, 19 bp, synthetic construct
  misc_feature 1..19: note = Description of Artificial Sequence: Synthetic primer
  source 1..19: mol_type = other DNA
tgacagttccttccacctg

SEQ ID NO: 258 - DNA, 19 bp, synthetic construct
  misc_feature 1..19: note = Description of Artificial Sequence: Synthetic primer
  source 1..19: mol_type = other DNA
tgtgaccttggactgtgtg

SEQ ID NO: 259 - DNA, 21 bp, synthetic construct
  misc_feature 1..21: note = Description of Artificial Sequence: Synthetic primer
  source 1..21: mol_type = other DNA
cactctgtgtccaatgcttac

SEQ ID NO: 260 - DNA, 21 bp, synthetic construct
  misc_feature 1..21: note = Description of Artificial Sequence: Synthetic primer
  source 1..21: mol_type = other DNA
tctggtataggcaagatcctg

SEQ ID NO: 261 - DNA, 20 bp, synthetic construct
  misc_feature 1..20: note = Description of Artificial Sequence: Synthetic primer
  source 1..20: mol_type = other DNA
aacttggttctcaactgcag

SEQ ID NO: 262 - DNA, 21 bp, synthetic construct
  misc_feature 1..21: note = Description of Artificial Sequence: Synthetic primer
  source 1..21: mol_type = other DNA
ctgactctgtgaacaatttgc

SEQ ID NO: 263 - DNA, 21 bp, synthetic construct
  misc_feature 1..21: note = Description of Artificial Sequence: Synthetic primer
  source 1..21: mol_type = other DNA
aatcttcacatcaattccctg

SEQ ID NO: 264 - DNA, 21 bp, synthetic construct
  misc_feature 1..21: note = Description of Artificial Sequence: Synthetic primer
  source 1..21: mol_type = other DNA
tgcattattgatagccatacg

SEQ ID NO: 265 - DNA, 21 bp, synthetic construct
  misc_feature 1..21: note = Description of Artificial Sequence: Synthetic primer
  source 1..21: mol_type = other DNA
tgccttacactggtacagatg

SEQ ID NO: 266 - DNA, 19 bp, synthetic construct
  misc_feature 1..19: note = Description of Artificial Sequence: Synthetic primer
  source 1..19: mol_type = other DNA
tataagcaaaggcctggtg

SEQ ID NO: 267 - DNA, 18 bp, synthetic construct
  misc_feature 1..18: note = Description of Artificial Sequence: Synthetic primer
  source 1..18: mol_type = other DNA
cgacagattcactcccag

SEQ ID NO: 268 - DNA, 20 bp, synthetic construct
  misc_feature 1..20: note = Description of Artificial Sequence: Synthetic primer
  source 1..20: mol_type = other DNA
ttcacttgccttgtaaccac

SEQ ID NO: 269 - DNA, 21 bp, synthetic construct
  misc_feature 1..21: note = Description of Artificial Sequence: Synthetic primer
  source 1..21: mol_type = other DNA
ctcactgtgtactgcaactcc

SEQ ID NO: 270 - DNA, 21 bp, synthetic construct
  misc_feature 1..21: note = Description of Artificial Sequence: Synthetic primer
  source 1..21: mol_type = other DNA
atgcacctattcagtctctgg

SEQ ID NO: 271 - DNA, 20 bp, synthetic construct
  misc_feature 1..20: note = Description of Artificial Sequence: Synthetic primer
  source 1..20: mol_type = other DNA
agagtgaaacctccttccac

SEQ ID NO: 272 - DNA, 18 bp, synthetic construct
  misc_feature 1..18: note = Description of Artificial Sequence: Synthetic primer
  source 1..18: mol_type = other DNA
agaagcatggtgaagcac

SEQ ID NO: 273 - DNA, 19 bp, synthetic construct
  misc_feature 1..19: note = Description of Artificial Sequence: Synthetic primer
  source 1..19: mol_type = other DNA
acctggctatggtacaagc

SEQ ID NO: 274 - DNA, 19 bp, synthetic construct
  misc_feature 1..19: note = Description of Artificial Sequence: Synthetic primer
  source 1..19: mol_type = other DNA
cagcaggcagatgattctc

SEQ ID NO: 275 - DNA, 21 bp, synthetic construct
  misc_feature 1..21: note = Description of Artificial Sequence: Synthetic primer
  source 1..21: mol_type = other DNA
tcaaccacttcagacagactg

SEQ ID NO: 276 - DNA, 23 bp, synthetic construct
  misc_feature 1..23: note = Description of Artificial Sequence: Synthetic primer
  source 1..23: mol_type = other DNA
attatatcacgtggtaccaacag

SEQ ID NO: 277 - DNA, 21 bp, synthetic construct
  misc_feature 1..21: note = Description of Artificial Sequence: Synthetic primer
  source 1..21: mol_type = other DNA
ggaggcggaaatattaaagac

SEQ ID NO: 278 - DNA, 20 bp, synthetic construct
  misc_feature 1..20: note = Description of Artificial Sequence: Synthetic primer
  source 1..20: mol_type = other DNA
ttgtttatgctgagctcagg

SEQ ID NO: 279 - DNA, 20 bp, synthetic construct
  misc_feature 1..20: note = Description of Artificial Sequence: Synthetic primer
  source 1..20: mol_type = other DNA
tacacagacagctcctccac

SEQ ID NO: 280 - DNA, 19 bp, synthetic construct
  misc_feature 1..19: note = Description of Artificial Sequence: Synthetic primer
  source 1..19: mol_type = other DNA
tggtaccgacaagatccag

SEQ ID NO: 281 - DNA, 22 bp, synthetic construct
  misc_feature 1..22: note = Description of Artificial Sequence: Synthetic primer
  source 1..22: mol_type = other DNA
tatgagaagcagaaaggaagac

SEQ ID NO: 282 - DNA, 20 bp, synthetic construct
  misc_feature 1..20: note = Description of Artificial Sequence: Synthetic primer
  source 1..20: mol_type = other DNA
gtcaacaccttcagcttctc

SEQ ID NO: 283 - DNA, 19 bp, synthetic construct
  misc_feature 1..19: note = Description of Artificial Sequence: Synthetic primer
  source 1..19: mol_type = other DNA
atcagaggttttgaggctg

SEQ ID NO: 284 - DNA, 19 bp, synthetic construct
  misc_feature 1..19: note = Description of Artificial Sequence: Synthetic primer
  source 1..19: mol_type = other DNA
aaccaaggactccagcttc

SEQ ID NO: 285 - DNA, 21 bp, synthetic construct
  misc_feature 1..21: note = Description of Artificial Sequence: Synthetic primer
  source 1..21: mol_type = other DNA
tttgaggctgaatttaagagg

SEQ ID NO: 286 - DNA, 21 bp, synthetic construct
  misc_feature 1..21: note = Description of Artificial Sequence: Synthetic primer
  source 1..21: mol_type = other DNA
gaaaccacttctttccacttg

SEQ ID NO: 287 - DNA, 21 bp, synthetic construct
  misc_feature 1..21: note = Description of Artificial Sequence: Synthetic primer
  source 1..21: mol_type = other DNA
tgttgctcttgaagtccatag

SEQ ID NO: 288 - DNA, 19 bp, synthetic construct
  misc_feature 1..19: note = Description of Artificial Sequence: Synthetic primer
  source 1..19: mol_type = other DNA
tggtatcgacaagacctgg

SEQ ID NO: 289 - DNA, 20 bp, synthetic construct
  misc_feature 1..20: note = Description of Artificial Sequence: Synthetic primer
  source 1..20: mol_type = other DNA
ggaacaccagtgactctgag

SEQ ID NO: 290 - DNA, 20 bp, synthetic construct
  misc_feature 1..20: note = Description of Artificial Sequence: Synthetic primer
  source 1..20: mol_type = other DNA
gactccactctcaagatcca

SEQ ID NO: 291 - DNA, 19 bp, synthetic construct
  misc_feature 1..19: note = Description of Artificial Sequence: Synthetic primer
  source 1..19: mol_type = other DNA
cyactctgargatccagcc

SEQ ID NO: 292 - DNA, 21 bp, synthetic construct
  misc_feature 1..21: note = Description of Artificial Sequence: Synthetic primer
  source 1..21: mol_type = other DNA
cattctgaactgaacatgagc

SEQ ID NO: 293 - DNA, 18 bp, synthetic construct
  misc_feature 1..18: note = Description of Artificial Sequence: Synthetic primer
  source 1..18: mol_type = other DNA
cttggagctggrsgactc

SEQ ID NO: 294 - DNA, 21 bp, synthetic construct
  misc_feature 1..21: note = Description of Artificial Sequence: Synthetic primer
  source 1..21: mol_type = other DNA
attctactctgaaggtgcagc

SEQ ID NO: 295 - DNA, 20 bp, synthetic construct
  misc_feature 1..20: note = Description of Artificial Sequence: Synthetic primer
  source 1..20: mol_type = other DNA
ataacttccaatccaggagg

SEQ ID NO: 296 - DNA, 20 bp, synthetic construct
  misc_feature 1..20: note = Description of Artificial Sequence: Synthetic primer
  source 1..20: mol_type = other DNA
ctgtagccttgagatccagg

SEQ ID NO: 297 - DNA, 19 bp, synthetic construct
  misc_feature 1..19: note = Description of Artificial Sequence: Synthetic primer
  source 1..19: mol_type = other DNA
tgttcactggtaccgacag

SEQ ID NO: 298 - DNA, 20 bp, synthetic construct
  misc_feature 1..20: note = Description of Artificial Sequence: Synthetic primer
  source 1..20: mol_type = other DNA
cgattttctgctgaatttcc

SEQ ID NO: 299 - DNA, 20 bp, synthetic construct
  misc_feature 1..20: note = Description of Artificial Sequence: Synthetic primer
  source 1..20: mol_type = other DNA
ttcctctcactgtgacatcg

SEQ ID NO: 300 - DNA, 20 bp, synthetic construct
  misc_feature 1..20: note = Description of Artificial Sequence: Synthetic primer
  source 1..20: mol_type = other DNA
ttcactctgaagatccggtc

SEQ ID NO: 301 - DNA, 20 bp, synthetic construct
  misc_feature 1..20: note = Description of Artificial Sequence: Synthetic primer
  source 1..20: mol_type = other DNA
actctgacagtgaccagtgc

SEQ ID NO: 302 - DNA, 19 bp, synthetic construct
  misc_feature 1..19: note = Description of Artificial Sequence: Synthetic primer
  source 1..19: mol_type = other DNA
gcaatcctgtcctcagaac

SEQ ID NO: 303 - DNA, 21 bp, synthetic construct
  misc_feature 1..21: note = Description of Artificial Sequence: Synthetic primer
  source 1..21: mol_type = other DNA
gatggatacagtgtctctcga

SEQ ID NO: 304 - DNA, 20 bp, synthetic construct
  misc_feature 1..20: note = Description of Artificial Sequence: Synthetic primer
  source 1..20: mol_type = other DNA
cagagaagggagatctttcc

SEQ ID NO: 305 - DNA, 20 bp, synthetic construct
  misc_feature 1..20: note = Description of Artificial Sequence: Synthetic primer
  source 1..20: mol_type = other DNA
ttcyccctgatyctggagtc

SEQ ID NO: 306 - DNA, 21 bp, synthetic construct
  misc_feature 1..21: note = Description of Artificial Sequence: Synthetic primer
  source 1..21: mol_type = other DNA
tctgactgtgagcaacatgag

SEQ ID NO: 307 - DNA, 21 bp, synthetic construct
  misc_feature 1..21: note = Description of Artificial Sequence: Synthetic primer
  source 1..21: mol_type = other DNA
agaatctctcagcctccagac

SEQ ID NO: 308 - DNA, 18 bp, synthetic construct
  misc_feature 1..18: note = Description of Artificial Sequence: Synthetic primer
  source 1..18: mol_type = other DNA
cctgcagccagaagactc

SEQ ID NO: 309 - DNA, 19 bp, synthetic construct
  misc_feature 1..19: note = Description of Artificial Sequence: Synthetic primer
  source 1..19: mol_type = other DNA
tctgagctgaatgtgaacg

SEQ ID NO: 310 - DNA, 20 bp, synthetic construct
  misc_feature 1..20: note = Description of Artificial Sequence: Synthetic primer
  source 1..20: mol_type = other DNA
gtgtrcccaggatatgaacc

SEQ ID NO: 311 - DNA, 21 bp, synthetic construct
  misc_feature 1..21: note = Description of Artificial Sequence: Synthetic primer
  source 1..21: mol_type = other DNA
tggttatagtgtctccagagc

SEQ ID NO: 312 - DNA, 20 bp, synthetic construct
  misc_feature 1..20: note = Description of Artificial Sequence: Synthetic primer
  source 1..20: mol_type = other DNA
tcyactctgamgwtccagcg

SEQ ID NO: 313 - DNA, 19 bp, synthetic construct
  misc_feature 1..19: note = Description of Artificial Sequence: Synthetic primer
  source 1..19: mol_type = other DNA
gtaccaacagagcctggac

SEQ ID NO: 314 - DNA, 20 bp, synthetic construct
  misc_feature 1..20: note = Description of Artificial Sequence: Synthetic primer
  source 1..20: mol_type = other DNA
ttctgatggctcaaacacag

SEQ ID NO: 315 - DNA, 23 bp, synthetic construct
  misc_feature 1..23: note = Description of Artificial Sequence: Synthetic primer
  source 1..23: mol_type = other DNA
atycgttcaaatatggaaagaaa

(19) United States
(12) Patent Application Publication
(10) Pub. No.: US 2026/0062696 A1
(43) Pub. Date: Mar. 5, 2026

(54) CLONING AND EXPRESSION SYSTEM FOR T-CELL RECEPTORS

(71) Applicant: St. Jude Children's Research Hospital, Inc., Memphis, TN (US)

(72) Inventors: Paul Thomas, Memphis, TN (US); Xizhi Guo, Memphis, TN (US); Pradyot Dash, Memphis, TN (US)

(73) Assignee: St. Jude Children's Research Hospital, Inc., Memphis, TN (US)

(21) Appl. No.: 19/172,537

(22) Filed: Apr. 7, 2025

Related U.S. Application Data
(62) Division of application No. 15/780,938, now Pat. No. 12,297,427.
(60) Provisional application No. 62/263,318, filed on Dec. 4, 2015.

(51) Int. Cl. C12N 15/10; C07K 14/725; C12N 15/66; C40B 50/06

(52) U.S. Cl. CPC C12N 15/1031; C07K 14/7051; C12N 15/10; C12N 15/1003; C12N 15/66; C12N 2310/11; C40B 50/06

(57) ABSTRACT

The invention provides a method for rapid cloning of T-cell receptors (TCRs) (e.g., paired αβ and γδ TCR chains) and B-cell receptors (BCRs) (e.g., paired IgH or IgK or Igλ) from single cells by CDR3 substitution using single cell PCR products and Gibson Assembly techniques and a pre-generated TCR (or BCR) library in an expression vector.

### CROSS REFERENCE TO RELATED APPLICATIONS

This present application is a Divisional of U.S. application Ser. No. 15/780,938, filed Jun. 1, 2018, which is a U.S. National Phase of International Patent Application No. PCT/US2016/064735, filed on Dec. 2, 2016, which published as WO 2017/096239 A1 on Jun. 8, 2017, and claims priority to U.S. Provisional Patent Application Ser. No. 62/263,318, filed on Dec. 4, 2015, all of which are herein incorporated by reference in their entireties.

### STATEMENT AS TO FEDERALLY SPONSORED RESEARCH

The invention was made with government support under grant AI107625 awarded by the National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Apr. 7, 2025, is named 243734_000215_SL.xml and is 376,760 bytes in size.

### FIELD OF THE INVENTION

The invention is directed to methods for rapid cloning and expression of T-cell receptors (TCRs) and B-cell receptors (BCRs) and their use for drug screening, structural and functional studies and other applications. More particularly, the invention provides a method for rapid cloning of TCRs (e.g., paired αβ and γδ TCR chains) or BCRs (e.g., paired IgH or Igk or Igh) from single cells by CDR3 substitution using single cell PCR products and Gibson Assembly techniques and a pre-generated TCR (or BCR) library in an expression vector.

### BACKGROUND OF THE INVENTION

T cells play a vital role in the control of viral infections and tumors. T cells are activated by antigen presenting cells via interactions between peptide-major histocompatibility complex (pMHC) and TCRs. This interaction can induce proliferation and the development of effector functions, including cytokine production and cytotoxic activity. T cells can also infiltrate infected or transformed tissues, e.g. as tumor-infiltrating lymphocytes (TILs), to perform these effector functions^{1,2}. However, in some chronic viral infections and tumors, responding effector T cells progressively get exhausted and become dysfunctional^{3,4,5}. In addition, control of tumors and/or infection may require large numbers of highly reactive lymphocytes that cannot be achieved due to normal tolerance mechanisms. One effective method to overcome this barrier is the use of therapeutic adoptive transfer of lymphocytes^{2,6,7}.

Adoptive transfer of lymphocytes such as in vitro expanded or TCR-engineered antigen specific T cells has been successfully used to control viruses and tumors in patients^{8,9,10,11,12}. In vitro expansion of viral or tumor-specific T cells require significant time to prepare and the targets are not usually fully characterized. Lymphocytes expressing engineered TCRs and chimeric antigen receptors (CAR) target specific antigens, with CARs recognizing surface antigens through immunoglobulin-type interactions^{10,13} and TCRs recognizing tumor-associated pMHC complexes. CAR therapy directed against surface antigens requires a tumor-associated antigen that can be universally targeted (even on healthy, non-tumor tissue) without significant toxicity. Tumor-specific antigens that are targeted by TCRs represent an attractive alternative that can provide greater specificity and reduce non-tumor associated toxicities^{14,15,16}. Additionally, engineered T cells expressing high-affinity antigen receptors can be conditioned to overcome immune tolerance, which has been a major limitation for immunotherapyl,^{14,15,17}. Apart from the clinical applications, a robust system for the cloning and expression of TCRs is a valuable tool for the investigation of TCR structure and functions^{18,19,20}.

Techniques to rapidly profile and clone antigen-specific TCRs have improved and shortened the process of TCR-engineered immunotherapy^{21,22}. These approaches are useful contributions to the field and are able to handle large cell inputs very effectively. However, for certain applications, the reported methods still have some limitations. First, approaches that rely on deep sequencing and cloning of bulk sorted cells can still be limited by target cell numbers. In contrast, single cell approaches can utilize input sizes starting with a single cell but are less efficient at dealing with high cell number inputs (greater than 10,000 cells). As a result, single cell methods are best directed at defined samples such as antigen-specific responses or tissue-associated infiltrating cells. Second, for bulk sorting, pairing of TCR chains requires algorithmic imputation, which can have difficulty dealing with cells expressing two distinct TCR chains of one type (e.g. two TCRa chains), which are quite common. A recently reported algorithm has addressed this concern by pairing bulk processed TCRs using barcoded pools of cells²³. However, this method requires relatively large inputs to successfully pair and would likely not be appropriate for very small sample sizes as might be obtained from tissue biopsies or tetramer sorting of small populations.

Third, while currently reported methods are able to generate full length receptors either by synthesis or by 5′ RACE-associated approaches at the single cell level, these methods require expansion of the isolated cells prior to TCR isolation, which likely causes bias in the TCR repertoire in the subsequent analyses and/or can reduce efficiency. Lastly, the majority of antiviral and antitumor adoptive therapy has focused on αβ T cell clones due to their exquisite antigen specificity. However, γδ T cells have also been shown to mediate antiviral and antitumor effects and are novel candidates for therapeutic development^{24,25}.

### SUMMARY OF THE INVENTION

To date, there is little research about profiling and utilizing the TCRγδ repertoire for therapeutic purposes. Applying γδ T cells for immunotherapeutic applications may be a promising future approach in conjunction with traditional TCRαβ techniques. Therefore, it is important to establish a system to define the repertoire and functional activity for γδ T cells. Additionally, improving efficiencies for cloning αβ TCRs from single cells may have complementary uses in the lab and in the clinic.

There is a great need in the art to develop a rapid, efficient and accurate cloning and expression method and/or system for specific TCRs, and uses thereof for screening TCR-mediated therapeutics, as well as for other research and/or clinical applications. The present invention fulfils such needs, and provides such methods and platforms.

In one aspect, the invention provides a method for cloning a T cell receptor (TCR) from a single T cell, wherein said method comprises:
(a) performing RT-PCR with a primer mixture on a single T cell to obtain paired αβ or γδ TCR CDR3 DNA sequences comprising a partial variable (V) region, CDR3 region, and a partial constant (C) region,
(b) optionally sequencing the RT-PCR product obtained in step (a), and
(c) cloning the αβ or γδ TCR CDR3 DNA sequences obtained in step (a) into a corresponding TCRαβ or TCRγδ library.

In one embodiment, said T cell is a human or a mouse αβ or γδ T cell.

In one embodiment, the method comprises sorting of single T cells prior to step (a). In one specific embodiment, T cells are not stimulated prior to sorting.

In one embodiment, the primer mixture comprises sense primers comprising T-cell receptor gamma variable (TRGV) sequences and/or T cell receptor delta variable (TRDV) sequences and antisense primers comprising T-cell receptor gamma constant (TRGC) sequences and/or T-cell receptor delta constant (TRDC) sequences. In one specific embodiment, the primer mixture comprises 9 TRGV and 8 TRDV sense primers and single TRGC and TRDC antisense primers. In another specific embodiment, the primer mixture comprises 5 external and 5 internal TRGV and 13 external and 13 internal TRDV sense primers and single TRGC and TRDC antisense primers. In one embodiment, the TRGV and/or TRDV sense primers and the TRGC and/or TRDC antisense primers are selected from the primers listed in Table 1. In one embodiment, the TRGV and/or TRDV sense primers and the TRGC and/or TRDC antisense primers are selected from the primers listed in Table 6.

In one embodiment, the single cell RT-PCR of γδ or αβ TCR and sequencing are performed within not more than 2 days.

In one embodiment, the method further comprises cloning the resulting αβ or γδ TCR CDR3 DNA sequences into the TCRαβ and/or TCRγδ library constructed using the method described below.

In a related aspect, the invention provides a method for constructing a TCRαβ and/or TCRyd library in an expression vector, comprising:
(a) synthesizing multiple pairs of TRGV and TRDV DNA fragments or TRAV and TRBV DNA fragments with a 15-25 bp overlap to the vector sequence based on the amplified sequence of the TRGV/TRDV or TRAV/TRBV pairings, respectively, and
(b) performing a two- or three-way ligation with a linearized expression vector.

In one embodiment of the above library construction method, the expression vector is a retroviral or lentiviral expression vector. In one embodiment, the ligation in step (b) is performed using Gibson Assembly Cloning techniques. In one specific embodiment, Gibson Assembly Cloning techniques are optimized to clone synthesized TRGV/TRAV and TRDV/TRBV DNA fragments using g-blocks or other synthesized DNA fragments (e.g., long primers that are then annealed). In one embodiment, the TCRαβ and/or TCRγδ library is constructed after a single-cell amplification and synthesized paired TRGV/TRDV or TRAV/TRBV receptors based on the sequence data. In one embodiment, the TCRαβ and/or TCRγδ library is constructed in 5 to 10 days. In one embodiment, the TCRαβ and/or TCRγδ library is used for drug screening or identification of TCRαβ- and/or TCRγ8-specific ligands.

In one embodiment of the above library construction, the method comprises substituting CDR3 regions of the existing clones in the TCRαβ and/or TCRγδ library with (i) products of RT-PCR performed using a primer mixture on a single T cell to obtain paired αβ or γδ TCR CDR3 DNA sequences comprising a partial variable (V) region, CDR3 region, and a partial constant (C) region and (ii) a linker DNA for overlap extension of PCR cloning. In one specific embodiment, said T cell is a human or a mouse αβ or γδ T cell. In one specific embodiment, the method comprises sorting of single T cells prior to RT-PCR. In one specific embodiment, T cells are not stimulated prior to sorting. In one specific embodiment, the primer mixture comprises sense primers comprising T-cell receptor gamma variable (TRGV) and/or T cell receptor delta variable (TRDV) and antisense primers comprising T-cell receptor gamma constant (TRGC) and/or T-cell receptor delta constant (TRDC) sequences. In one specific embodiment, the primer mixture comprises 9 TRGV and 8 TRDV sense primers and single TRGC and TRDC antisense primers. In one specific embodiment, the primer mixture comprises 5 external and 5 internal TRGV and 13 external and 13 internal TRDV sense primers and single TRGC and TRDC antisense primers. In one specific embodiment, the TRGV and/or TRDV sense primers and the TRGC and/or TRDC antisense primers are selected from the primers listed in Table 1. In one specific embodiment, the TRGV and/or TRDV sense primers and the TRGC and/or TRDC antisense primers are selected from the primers listed in Table 6. In one specific embodiment, said linkers are overlapping with the non-variant sequences of the TCRα/γ and TCRβ/δ single cell RT-PCR products. In one specific embodiment, said linker sequences are selected from those listed in Table 3. In one specific embodiment, the resulting TCRαβ and/or TCRγδ chains with CDR3 substitutions are used for T cell-mediated immunotherapy.

In a related aspect, the invention provides a TCRαβ and/or TCRγδ library constructed using any of the above methods. In another related aspect, the invention provides a host cell (e.g., a Nur-77-GFP Jurkat 76 cell or a Nur-77-Luciferase Jurkat 76 cell) comprising said TCRαβ and/or TCRγδ library construct.

In another aspect, the invention provides a method for cloning a B cell receptor (BCR) from a single B cell, wherein said method comprises:
(a) performing RT-PCR with a primer mixture on a single B cell to obtain paired IgH or Igk or Igλ CDR3 DNA sequences comprising a partial variable (V) region, CDR3 region, and a partial constant (C) region,
(b) optionally sequencing the RT-PCR product obtained in step (a), and
(c) cloning the IgH or Igκ or Igλ CDR3 DNA sequences obtained in step (a) into a corresponding BCR library.

In one embodiment, said B cell is a human or a mouse B cell. In one embodiment, the method comprises sorting of single B cells prior to step (a).

These and other aspects of the present invention will be apparent to those of ordinary skill in the art in the following description, claims and drawings.

### BRIEF DESCRIPTION OF THE DRAWINGS

FIG. 1A shows a schematic of unbiased single-cell amplification of paired TCR CDR3 regions. Overview of the multiplex PCR protocol to amplify and sequence paired TCR CDR3 α/γ and CDR3 β/δ. After sorting single human αβ or γδ T cells into a 96-well plate, reverse transcription is performed to obtain single-cell cDNA. Taking human γδ T cells as an example, a first round of PCR is performed by using an external primer mixture of 9 TRGV and 8 TRDV sense and single TRGC and TRDC antisense primers following RT-PCR. The first-round PCR products are subjected to two separate second-round PCRs using a corresponding internal primers mix (9 sense TRGV, single antisense TRGC, and 8 sense TRDV, single antisense TRDC, respectively). The timeline of this process is shown on the left.

FIG. 1B shows an agarose gel electrophoresis image of TCR segments containing CDR3γ and CDR3δ is shown. Paired CDR3γ and CDR3δ products from the same cell were loaded in adjacent lanes. Negative control PCR reactions are shown in the boxed region and in the ladder lane, a 500 bp label is shown.

FIG. 1C shows the determination of paired TRGV/TRDV usageby multiplex PCR and sequencing (n=14 human apheresis rings), with the percentage of different TRGV/TRDV usage in each sample assessed (mean±SEM).

FIG. 2A shows a schematic of rapid cloning and expression of human TCRαβ or TCRγδ in a retroviral vector. A schematic diagram of TCR cloning using gBlock® synthesized DNA fragments and a linearized retroviral vector (pMICherry) is shown. Family specific TRGV and TRDV full length TCR chains were synthesized with a 15-20 bp overlap sequence (light diagonal line shading) in the 2A region. Together with a linearized pMICherry expression vector, a three-way ligation is performed by using Gibson Assembly® Cloning. The timeline of this process is presented on the left.

FIG. 2B shows expression of TCR constructs in the Jurkat 76 TCRα⁻β⁻ cell line. The vectors with human TRGV9/TRDV2 TCR genes and human influenza-specific TCRαβ genes were co-transfected with the human CD3 construct into the Jurkat 76 TCRα⁻β⁻ cell line. The flow cytometry results of transfected cells are shown.

FIG. 3A illustrates how Nur77-GFP Jurkat 76 TCRα⁻β⁻ cells can report the TCR signaling activation. Following co-transfection of a murine K^{b}PB1₇₀₃-specific TCR αβ derived from influenza-infected mice and a mouse CD3 construct, the K^{b}PB1₇₀₃⁺ TCRαβ⁺ NJ76 cells (PB1-NJ76) were stimulated either with influenza PB1₇₀₃ peptide alone or PB1₇₀₃ peptide-pulsed splenotytes for 4 hrs. Anti-mouse CD3/α-human CD28 stimulation was also done as a positive control. The GFP expression was assessed by flow cytometry.

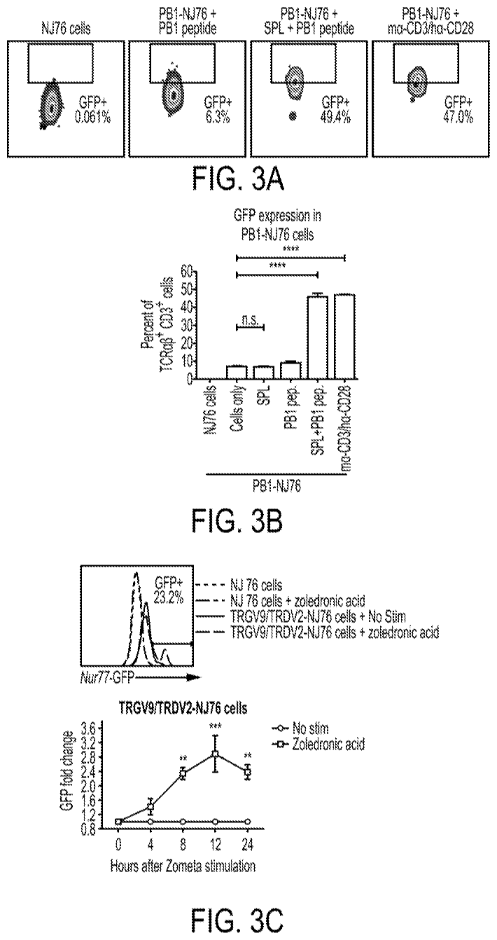

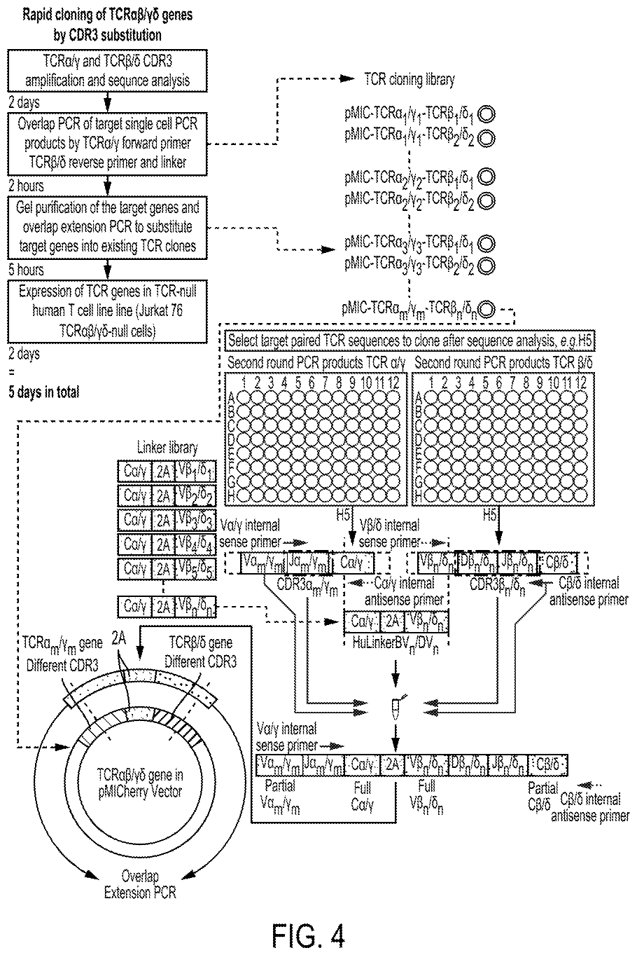

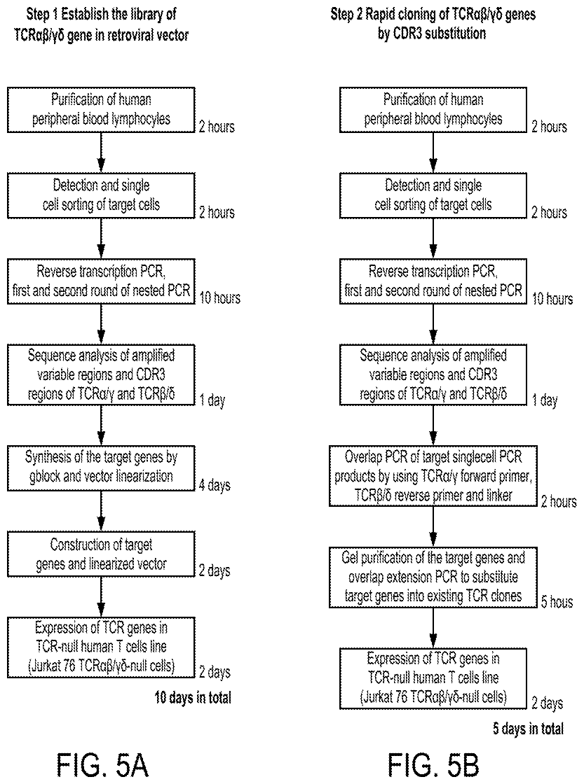

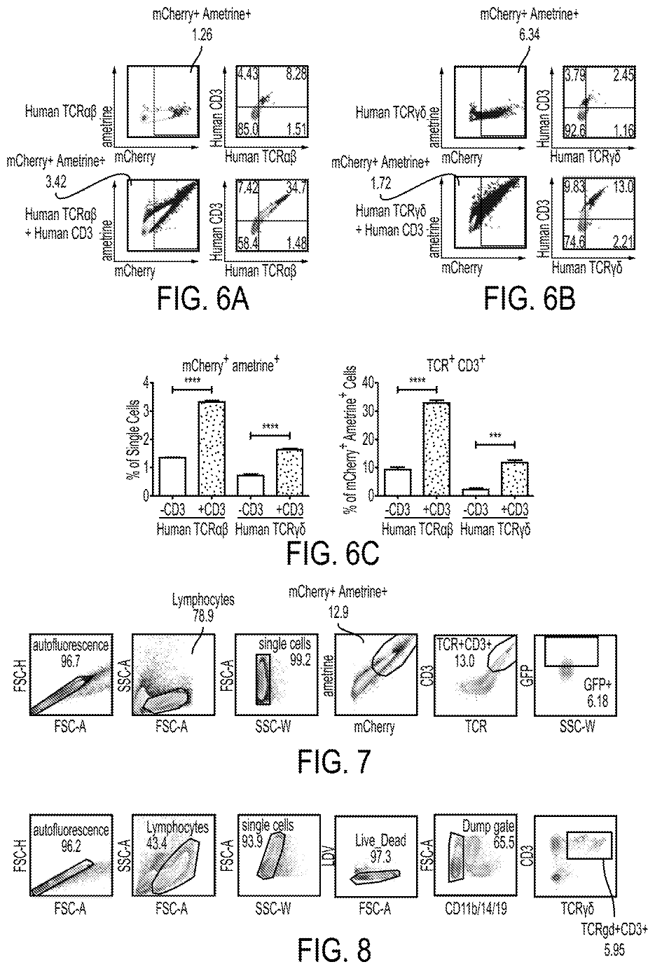

In FIG. 3B, the quantification of GFP expression in PB1-NJ76 cells is shown. Statistical differences were determined by One-way ANOVA.

In FIG. 3C, the transfected human TRGV9/TRDV2-NJ76 cells were pulsed with zoledronic acid (50 ug/ml) for 3 h at 37° C., and washed and incubated at 37° C. for 12 h. The GFP expression of stimulated NJ76 cells, non-stimulated TRGV9/TRDV2-NJ76 and stimulated TRGV9/TRDV2-NJ76 cells with zoledronic acid is shown in the top panel. Fold change of GFP expression in stimulated TRGV9/TRDV2-NJ76 cells with zoledronic acid compared to non-stimulated TRGV9/TRDV2-NJ76 is shown as a time course in the bottom panel. Statistical differences were determined by Two-way ANOVA; p<0.05 was considered statistically significant. Data are mean±SEM of two independent experiments. **p<0.01, ***p<0.001, ****p<0.0001, n.s. non-significant.

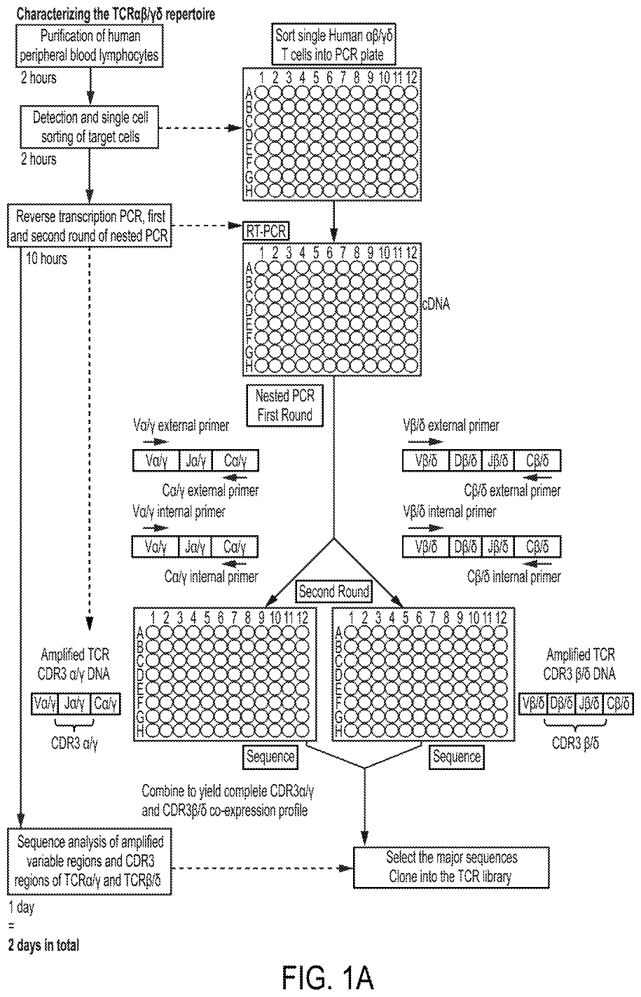

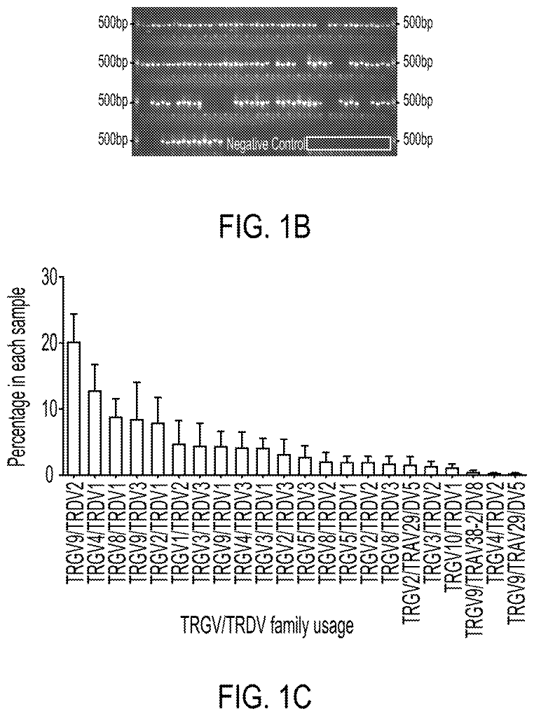

FIG. 4 shows a schematic strategy of CDR3 substitution by overlap extension PCR. Based on the library of TCRαβ and TCRγδ established by the described cloning platform, the strategy for CDR3 substitution using multiplex single cell PCR products and linker DNA is shown. After the sequence analysis of single-cell PCR (shown in FIG. 1A), the target pairs of TCRs are chosen from the respective second round paired PCR plates, which include TCRα/γₘ and TCRβ/δₙ (m represents a particular TRAV or TRGV subfamily; n represents a particular TRBV or TRDV subfamily). Beforehand, we generated a library of linker DNA by gBlock synthesis (IDT) (Table 3). The linker DNA consists of TRAC/TRGC-2A-TRBVn/TRDVn (n represents the TRB/DV subfamily) sequence. Using the single cell PCR products of α/γ and β/δ chains of the desired clonotypes and the relevant linker gBlock DNA, we carried out an overlap PCR with TRAm/GVm internal sense primer and TRB/DC internal antisense primer. The PCR products were visualized on an agarose gel, and subsequently purified to use as “mega-primer” for cloning into the existing construct from our TCR cloning library (pMIC-TCRα/γₘ-TCR β/δₙ) with the same TRGV and TRDV family usage but different CDR3s by overlap extension PCR. The timeline of the whole process is on the left.

FIGS. 5A and 5B depict the time lines of the two cloning platforms.

FIGS. 6A-6C show co-transfection of human CD3 can improve the expression of human TCR constructs. FIGS. 6A and 6B show a copmarison of single transfection of human TCR constructs and co-transfection of human TCR constructs and human CD3. FIG. 6C shows quantification of mCherry/ametrine and TCR/CD3 expression is shown. Statistical differences were determined by One-way ANOVA; p<0.05 was considered statistically significant. Data are mean±SEM of two independent experiments. ***p<0.001, ****p<0.0001.

FIG. 7 shows a gating strategy of TCR-transfected-NJ76 cells in flow cytometry. The data of TCR-transfected-NJ76 cells after stimulation in FIGS. 3A-3C were analyzed by applying the gating strategy to all the samples. The gating is flowing “autofluorescence gate—lymphocytes gate—single cell gate—mCherry+Ametrine+gate—TCR+CD3+gate—GFP+gate”.

FIG. 8 shows a gating strategy of Human TCRγ/δ⁺ CD3⁺ cells single cell sorting. Single cells of human TCRγδ⁺ CD3⁺ cells from PBMC samples were sorted into 96-well plate by applying the gating strategy of FIG. 7, above. The gating is flowing “autofluorescence gate—lymphocytes gate—single cell gate—live/dead gate—dump gate (CD11b/14/19)-TCRγδ⁺/CD3⁺ gate”.

### DETAILED DESCRIPTION OF THE INVENTION

The present invention provides a rapid, efficient and accurate cloning and expression method and system for specific TCRs (e.g., paired αβ and γδ TCR chains from single cells) which can be used for drug screening (e.g., for T cell-mediated anti-tumor or anti-infective immunotherapy), for structuring and functional analysis of TCRs, and other applications. The invention addresses the non-specific, labor-intensive and time-consuming issues of prior PCR-based cloning methods and provides a high-throughput, accurate and efficient method of TCR engineering for therapeutic and research applications.

In conjunction with single cell multiplex PCR techniques for TCRαβ or TCRγδ profiling 26.27, and Gibson Assembly® cloning of synthesized DNA, the invention provides rapid sequencing and cloning of specific TCRs in an expression vector (e.g., retroviral expression vector). By generating TCR libraries, the invention provides a cloning and expression method and system that is significantly accelerated by only requiring the substitution of the CDR3 region, resulting in TCR clones in appropriate expression vectors in as little as five days after cell isolation. The invention provides highly robust, inexpensive, efficient, and high-throughput means for TCR engineering for therapeutic and research applications.

In certain embodiments, the invention provides a method of single-cell amplification of paired TCR CDR3 α/γ and CDR3 β/δ regions comprising the steps of (a) sorting of single human αβ or γδ T cells; (b) performing RT-PCR to obtain a single-cell cDNA; and (c) amplifying the single-cell cDNA obtained in step (b) in a second round PCR with a primer mixture of TRGV and TRDV sense primers and TRGC and TRDC antisense primers. In one embodiment, the single-cell cDNAs are amplified with a primer mixture comprising nine (9) T-cell receptor gamma variable (TRGV) and/or eight (8) T-cell receptor delta variable (TRDV) sense primers and a single T-cell receptor gamma constant (TRGC) and/or T-cell receptor delta constant (TRDC) antisense primer. Non-limiting examples of external and internal sense primers targeting TRGV and TRDV and antisense TRGC and TRDC primers are listed in Table 1, below. Specific non-limiting examples of a method for sorting single cells, as well as conditions for RT-PCR and nested PCRs, are provided below (see Example 1, Materials & Methods).

In certain embodiments, the invention also provides a method of production of TCRαβ and TCRγδ library in an expression vector, comprising the steps of (a) synthesizing multiple pairs of TRGV and TRDV DNA fragments with a 15-20 bp overlap in the sequence in the 2A region based on the TRGV/TRDV usage in human apheresis ring samples, and (b) performing a three-way ligation with a linearized expression vector (e.g., a retroviral vector, which is convenient for the future applications, like transduction of cell lines and TCR-transgenic mice) using Gibson AssemblyR Cloning or another type of ligation, including, e.g., conventional T4-mediated ligation. In certain embodiments, the TCRαβ and/or TCRγδ library is human library comprising the human TCRαβ and TCRγδ. In certain embodiments, the production of TCRαβ and TCRγδ library is performed after the single-cell amplification and paired TRGV/TRDV usage based on the sequence data. Exemplary primers targeting the 2A regions of human CD3δ, γ, ε and ζ genes are provided in Tables 2A & 2B.

The Gibson Assembly kit is an enzyme and buffer mix designed to optimize the overlap ligation of g-block gene fragments. In certain embodiments, Gibson Assembly protocol is optimized to synthesize the TRGV and TRDV DNA fragments or genes using g-block technique, IDT DNA). However, the ligation reaction can be performed using other suitable ligases known and available in the art. Using the methods of the present invention, a TCR library can be constructed in as fast as 10 days. As long as the library is established, it can be applied to substitute CDR3 regions from a new patient sample, which can dramatically reduce the cloning time from 10 days to 5 days. This could be beneficial for both adoptive transfer therapy and personalized therapy.

The invention provides a rapid cloning method based on the TCR library constructed, e.g., the TCRαβ and TCRγδ library, comprising the step of generating full-length paired αβ or γδ TCR chains by CDR3 substitution using multiplex PCR products and a linker DNA (overlap extension PCR). In certain embodiments, the linkers contribute to simultaneously substitute CDR3 regions of both TCRα/γ and TCRβ/δ. The linkers are overlapping with the single cell PCR products, therefore, no additional PCR steps are needed. In certain embodiments, the TCRα/γ and TCRβ/δ single cell PCR products and the linkers are mixed together and an overlap PCRs are performed, and then the PCR products generated from the overlap PCRs are used to substitute the CDR3 regions in the cloning library. Exemplary human TCRγδ linker sequences are provided in Table 3, below.

The methods of the present inventions improve the speed and specificity for cloning paired TCRs. With single cell analyses, the cloned paired TCRs of the present invention are more “specific” than examining T cell receptors in bulk because both chains of the receptor are from the same cell. The invention allows rapid cloning of TCRs that are responding to an infection or a tumor, which could be useful in developing targeted cell therapies, e.g., by reintroducing those receptors into the patient's cells to assist in mounting a more effective response. The TCR sequences themselves can also be useful for understanding what targets the response is against. Although there is no simple way of decoding the target from the TCR sequence, databases and/or computer algorithms can be developed to evaluate the relationship on the antigens and the elicited T-cell responses via various TCRs. Furthermore, the invention provides an efficient and accurate TCR expression system for TCRs in a reporter cell line, which can be used for screening specific antigens directly. TCR sequencing can also be used for the detection of minimal residual disease in leukemia and lymphoma. Many tumors have rearranged their TCRs. In these cases, the specific TCR sequence in the tumor becomes a lineage “barcode” for the tumor itself. The methods of the invention can provide TCR sequencing information to determine the presence and/or quantity of tumor cells.

### Definitions

In describing the present invention, the following terms will be employed, and are intended to be defined as indicated below.

“CDR3 region” or “the third complementarity determining region” is defined herein as the region from codon positions 105 to the end of the V-REGION in germline gDNA or cDNA, codon positions 105 to 117 in V-DOMAIN of rearranged gDNA or in cDNA (all the position numbers are according to the IMGT unique numbering; see Lefranc, M.-P., The Immunologist, 7, 132-136 (1999) and www.imgt.org/IMGTScientificChart/Nomenclature/IMGT-FRCDRdefinition.html).

As used herein the term “partial variable region” or “partial V region” is determined by the position of the internal forward primers targeting the amplified V-region of interest (e.g., α, β, γ, δ; see, e.g., Tables 8-11) until the codon position 105 (all the position numbers are according to the IMGT unique numbering; see Lefranc, M.-P., The Immunologist, 7, 132-136 (1999) and www.imgt.org/IMGTScientificChart/Nomenclature/IMGT-FRCDRdefinition.html).

As used herein the term “partial constant region” or “partial C region” refers to a region that includes the first codon of the C region until a position defined by the reverse internal primer used to amplify the C region of interest (e.g., α, β, γ, δ; see Tables 8-11).

The term “corresponding TCR library” means the same variable (V) family usage.

It must be noted that, as used in this specification and the appended claims, the singular forms “a,” “an” and “the” include plural referents unless the content clearly dictates otherwise.

The terms “subject,” “individual,” and “patient,” are used interchangeably herein and refer to any mammalian subject for whom diagnosis, prognosis, treatment, or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, and hamsters; primates, and transgenic animals.

The term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, preferably up to +10%, more preferably up to +5%, and more preferably still up to +1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A practical Approach, Volumes I and II (D. N. Glover ed. 1985); Oligonucleotide Synthesis (MJ. Gait ed. 1984); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; B. Perbal, A practical Guide To Molecular Cloning (1984); F. M. Ausubel et al. (eds.), Current Protocols in Molecular Biology, John Wiley & Sons, Inc. (1994); among others.

### Method and Platform of the Invention

The present invention provides several useful techniques for the analysis of TCR biology, including a single cell based protocol for γδ TCR amplification, a rapid protocol for TCR cloning and expression, and a novel platform for functional characterization of TCR clones (see FIGS. 5A and 5B). The invention provides an accurate and efficient method to approach rapid TCR cloning at the single cell level, which can improve the development of multiple applications, including TCR-mediated immunotherapy.

The most prominent recent immunotherapy approaches involve T cell checkpoint blockade inhibitors⁵¹. However, these therapies depend on the presence of significant numbers of anti-tumor T cell responses. The ex vivo expansion of tumor infiltrating lymphocytes has also been successful, but is time consuming⁵². The method of the present invention significantly accelerates the amount of time needed to generate large numbers of anti-tumor T cells, by allowing the efficient transduction of identified anti-tumor receptors. The key to the application of the invention of directed T cell immunotherapy is the rapid and accurate isolation and cloning of paired TCRs. Thus far, various methods have been developed for the cloning of TCR genes by traditional PCR, but the acquisition and expression of TCRs is often labor-intensive, time-consuming, expensive, and non-specific. The present invention provides efficient acquisition of TCR gene products for cloning based on single cell isolation, with an amplification success rate of isolated paired single cell TCRγ and TCRδ CDR3 products of 71,25±18.75% based on total sorted single cells in each sample.

The invention also provides a platform for screening TCR activation after cloning. By inserting the Nur77 reporter into a Jurkat 76 TCRα-β-cell line, a useful system for monitoring specific TCR activation was generated, as demonstrated by stimulation of PB1-NJ76 by its cognate influenza-derived peptide and stimulation of TRGV9/TRDV2 T cells with, e.g., zoledronic acid.

In certain embodiments, the invention employs the insertion of Nur77-GFP BAC DNA into the Jurkat cell line as a reporter cell line. Prior approaches to detect T cell activation include CD69 expression on the cell surface²², and detection of IFNγ, IL-2, and TNFα protein levels in cell culture supernatants by ELISA^{21,22}. These conventional methods have some shortcomings. For instance, CD69 is common activation marker of T cells, so it cannot show the specific activation of T cells through TCR signaling-bystander activation can occur. Detection of IFNγ, IL-2 and TNFα by ELISA is time-consuming (2 days) and expensive, and Detection of IFNγ, IL-2 and TNFα by real-time is labor-intensive (requires RNA isolation), time-consuming (at least a day).

The Nur77-GFP system has been demonstrated to reflect specific TCR triggering after stimulation^{40,41,42}, instead of activation by other receptors on the cell surface, like TLRs, NKG2A/2D, or other inhibitory receptors. Furthermore, GFP is directly assayable by flow cytometry without any secondary processing. The Nur77-GFP system allows rapid and accurate detection of specific T cell stimulation in a high-throughput manner. In certain embodiments, the invention provides that zoledronic acid induces the GFP expression of TRGV9/TRDV2 Nur77-GFP cells, demonstrating that the invention platform is functional, and zoledronic acid can be used in combination with TRGV9/TRDV2-expressing cells as a positive control for the test platform and that peptides can be used in combination with their cognate TRBV/TRAV-expressing cells (as demonstrated in FIG. 3B) as a control for the test platform.

Therefore, the present invention provides a novel platform and/or system that can be used to test different molecules directly by stimulating the Nur77-GFP Jurkat cell line, and characterizing and quantifying the stimulation based on GFP expression by flow cytometry. The platform of the present invention is faster, easier and more inexpensive (no need to stain) to perform, and can be used to screen TCR-activating or modifying drugs in a high-throughput screening manner as compared to prior art platforms.

In other embodiments, a Nur77-Luciferase reporter, which has a lower signal to noise ratio, is included in the Nur77-GFP Jurkat cell line to decrease the background for certain types of drug screening. Insertion of Nur77-Luciferase BAC DNA into the Jurkat cell line instead of Nur77-GFP can be undertaken, followed by luciferase detection in various assays that include high-throughput screening platforms. The luciferase detection may be conducted with between 5×10⁴ to 2.5×10⁵ cells per well, or about 1×10⁵ cells per well, for highly sensitive detection.

Another application of the Nur77-GFP system is to screen for compounds that inhibit T cell activation. The Nur77-GFP system of the invention can be used for drug screening, clinical applications, and basic immunology research.

Considering the variability of CDR3 sequences and TCR variable regions (approximately 1018 combinations in human TCRγδ cells and 10¹⁶ combinations for TCRαβ cells⁵³) and the complexity of cloning all the different clones de novo, the invention provides a method using overlap extension PCR of a linker molecule with amplified single cell CDR3 products and a constructed γδ (or αβ) TCR library to rapidly (less than 5 days) generate diverse TCR clones. Setting up a TCR clone library with all possible combination of TCRγ/TCRô or TCRβ/TCRα pairings and a DNA linker library allows the achievement of superior speed by overcoming the need to synthesize the hypervariable CDR3 portion of the DNA. In the method of the present invention, as soon as the sequence information of the TCR CDR3 region from the single-cell PCR and their family usage are known, the relevant clone can be picked up from the library with the required TRBV and TRAV families to use as a backbone for the final construct. The single-cell PCR products of TCRβ/γ and TCRα/δ can then be linked together by using two terminal primers and a DNA linker by overlapping PCR. Next, this target gene can be substituted directly into the existing clones and the irrelevant CDR3s can be replaced with the specific ones while preserving the family usage. In certain embodiments, new CDR3s of TRGV9-TRDV2 are put into the existing cloning of TRGV9-TRDV2 vector with different CDR3s by overlapping extension PCR. With the method of the present invention, cloning TCR genes can be achieved within 5 days.

As compared to prior art methods^{21,22}, important advantages of the methods of the present invention are as follows:
1. The method of the present invention provides a way to build a library of clones as a one-time necessity that can serve as a backbone to the second, rapid TCR cloning method.
2. The method of the present invention does not require stimulation of PBMCs (e.g., with PHA/IL-2), which saves at least 1 day (and potential variability and survival issues) and helps avoid bias in the TCR repertoire in the subsequent analyses (as stimulation of PBMCs may cause unnecessary selection of TCR repertoire due to expansion of a subset of T cells, as well as a high rate and/or non-physiological level of dual TCRα and dual TCRβ expression in a single cell due to clonal expansion under the stimulation).
3. The method of the present invention is substantially faster than the reported approaches and relies on more robust processes. Once the library is made, the invention platform only needs 5 days to clone TCRs. Prior approaches are more labor intensive because they clone the full-length TCR de novo. In contrast, the method of the present invention uses the single-cell PCR products to clone any pairs of TCRs, and the libraries constructed using these clones can be continuously used.
4. The method of the present invention can yield an average 71% of TCR pairs from human samples (compare with an average 34% yield reported in 22 or 147 T cells in samples from 61 patients identified in 21).
5. While in prior methods²² TCRα/γ and TCRβ/δ were inserted into different vectors to transfect cells, which may cause the biased expression of TCRs, in the method of the present invention TCRα/γ and TCRβ/δ are inserted under the same promoter separated by a 2A sequence to ensure their equal level of expression.
6. In certain embodiments, the method of the present invention uses Jurkat 76 cell line (the TCR-negative human T cell line) to express human TCR clones. Such human cell line likely more accurately mimics human T cells and is more amenable to adoptive transfer therapy than mouse cell lines used in prior art.
7. The single-cell technique used in the method of the present invention allows to examine small number of cells (as low as 1) which is important e.g., in solid tumors where the numbers of TILs are limiting.

### Uses of the Methods of the Invention

The rapid TCR cloning method of the present invention is very useful for immunotherapy. Tumor-specific T cells have been characterized by broad non-specific surface phenotypes that can be used to isolate, clone, and express potential tumor-targeted clones⁵⁴. The recent advancement of tumor sequencing has allowed for identification of tumor neoantigens and overexpressed self-antigens^{55,56,57,58}. Combining these technologies will allow characterization and tailoring of anti-tumor therapy.

T cell transfers have also been used for the treatment of opportunistic infections in immunosuppressed patients, particularly after hematopoietic stem cell transplant. The reactivation of herpes viruses like human cytomegalovirus and Epstein-Barr virus is a clinical dilemma that cannot always be addressed with antivirals^{24,59,60}. Analogous to TIL therapies, ex vivo expansion of antiviral T cell specificities can be clinically useful, but suffers from similar workflow limitations. By generating a library of specific TCR constructs reactive against a range of viruses and HLA types, TCR-directed therapies could be used prophylactically or immediately at the earliest signs of reactivation.

In addition to these therapeutic applications, the method of the present invention significantly improves the workflow for cloning and expressing TCRs for study in vitro. This can include the characterization of biochemical features of the TCR-peptide-MHC interaction, or, in the case where ligands have not been identified transduced cell lines can be used for the screening of novel antigens. This is particularly useful in the context of γδ T cells, where very few ligands have been identified and confirmed^{18,61}. The GFP reporter line engineered can be used directly in high-throughput screening platforms; alternative reporters (such as, e.g., luciferase) can be easily substituted as well.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1

### Materials and Methods

Subjects and peripheral blood mononuclear cell (PBMC) samples. Samples were obtained using research protocols approved by St. Jude Children's Research Hospital's institutional review boards (IRB) (Memphis, TN). Peripheral whole blood was collected from heparinized apheresis rings from healthy immunocompetent individuals not taking immunomodulatory pharmaceutical agents. PBMCs were isolated via density gradient centrifugation (GE Healthcare Ficoll-Paque PLUS), and red blood cells (RBCs) were removed using RBC lysis buffer (8.3 g NH₄Cl, 1 g KHCO₃, and 1 ml 0.1% Phenol Red in 1 L distilled water). Isolated PBMCs were frozen in −80° C. for future use. All PBMCs used in the paper were stored frozen. Compared to fresh PBMC data from healthy apheresis rings, frozen PBMCs did not have a significantly lower success rate for single-cell amplification.

Single cell sorting and staining. PBMCs were treated with human FcR blocking reagent (Miltenyi Biotec) on ice for 20 minutes. Human TCRγδ cells were isolated by staining with PE-conjugated anti-human TCRγ/δ (Biolegend, clone: B1), FITC-conjugated anti-human CD3 (Biolegend, clone: OKT3), a dump gate consisting of APC-conjugated anti-human CD11b/CD14/CD19 (Biolegend, CD11b clone: ICRF44; Biolegend, CD14 clone: HCD14; Biolegend, CD19 clone: HIB19) and Live/Dead Violet exclusion dye (Invitrogen, L34955) on ice for 30 minutes. After staining, TCRγ/δ CD3 cells were sorted directly into a 96-well PCR plate (Biorad) with a sorter (Model sy3200, Sony Biotech Synergy sorter) by the following gating strategy: autofluorescence gate—lymphocytes gate—single cell gate—live/dead gate-dump gate (CD11b/14/19)—TCRγδ/CD3 gate (FIG. 8). The last 2 columns of the plate were left empty for use as PCR negative controls. After sorting, plates were stored at −80° C. until downstream processing. Human αβ T cells were also isolated using the same method²⁷, which consisted of APC-conjugated anti-human CD14/CD19/CD11b (Biolegend, CD14 clone: HCD14; Biolegend, CD19 clone: HIB19; Biolegend, CD11b clone: ICRF44), PE-conjugated anti-human TCRα/β (Biolegend, clone: IP26), FITC-conjugated anti-human CD3 (Biolegend, clone: OKT3) and Live/Dead Violet L34955 (Invitrogen). To increase PCR efficiency, the plates were pre-loaded with mixture of RT-PCR by SuperScript® VILO cDNA synthesis kit and single cells were sorted directly into these plates.

Reverse transcription, multiplex, nested single cell PCR, and sequencing. cDNA from TCRγδ and TCRαβ mRNA was reverse transcribed directly from the sorted and stored single cells in the PCR plate without any RNA extraction step using the iScript cDNA Synthesis Kit (Bio-Rad) in a 2.5 μl reaction mix as per the method described previously²⁶. The cDNA synthesis was carried out by incubating at 25° C. for 5 min, 42° C. for 30 min, and 80° C. for 5 min (For the first round and second round of PCR). Alternatively, the SuperScript® VILO cDNA synthesis kit was used which produces a higher success rate for single cell PCR by incubating the reaction mixture at 25° C. for 10 min, 42° C. for 60 min, and 80° C. for 5 min. The TCRαβ transcripts from each cell were amplified by a multiplex nested PCR strategy as described previously^{26,27}. For amplification of TCRγδ transcripts, the overall strategy was similar to the published TCRαβ amplification (95° C. for 2 min, followed by 35 cycles of 95° C. for 20 s, 53° C. for 20 s, and 72° C. for 45 s, followed by final extension of 72° C. for 7 min.), except for the primers described in Table 1.

TABLE 1
Primers targeting human T cells receptor
gamma (TRGV) and delta (TRDV) genes

| TRGV gene(s) | | |
|---|---|---|
| targeted by | External | Internal |
| primer | primer sequence | primer sequence |
| HuTRGV3.5 | 5′TCTTCCAACTTGGAAGGG3′ | 5′GGTCATCTGCTGAAATCAC3′ |
| | (SEQ ID NO: 1) | (SEQ ID NO: 2) |
| HuTRGV7 | 5′TCTTCCAACTTGCAAGGG3′ | 5′GGTCATCTGCTGTAATCACTTG3′ |
| | (SEQ ID NO: 3) | (SEQ ID NO: 4) |
| HuTRGVA | 5′GGGTCATCCTGTTTCCAG3′ | 5′TACCTAAGGACCTGTGTAGAGG3′ |
| | (SEQ ID NO: 5) | (SEQ ID NO: 6) |
| HuTRGVB | 5′TGGCCTCCCAAAGTACTG3′ | 5′TCCTCTTTCTATGTCCCAGG3′ |
| | (SEQ ID NO: 7) | (SEQ ID NO: 8) |
| HuTRGV8 | 5′CCAACTTGGAAGGGAGAAC3′ | 5′AAAATGCCGTCTACACCC3′ |
| | (SEQ ID NO: 9) | (SEQ ID NO: 10) |
| HuTRGV9 | 5′CCAGGTCACCTAGAGCAAC3′ | 5′TGTCCATTTCATATGACGG3′ |
| | (SEQ ID NO: 11) | (SEQ ID NO: 12) |
| HuTRGV10 | 5′TTATCAAAAGTGGAGCAGTTC3′ | 5′CAGCTATCCATTTCCACGG3′ |
| | (SEQ ID NO: 13) | (SEQ ID NO: 14) |
| HuTRGV11 | 5′GAACAACCTGAAATATCTATTTCC3′ | 5′CATATCTTGGAAGGCATCC3′ |
| | (SEQ ID NO: 15) | (SEQ ID NO: 16) |
| HuTRGV1.2.4.6 | 5′GGGTCATCTGCTGAAATCAC3′ | 5′CCAGGAGGGGAAGGC3′ |
| | (SEQ ID NO: 17) | (SEQ ID NO: 18) |
| HuTRGC | 5′GGTGTTCCCCTCCTGG3′ | 5′CCCAGAATCGTGTTGCT3′ |
| | (SEQ ID NO: 19) | (SEQ ID NO: 20) |
| HuTRDV1 | 5′GCCCAGAAGGTTACTCAAG3′ | S′AGCAAAGAGATGATTTTCCTTA3′ |
| | (SEQ ID NO: 21) | (SEQ ID NO: 22) |
| HuTRDV2 | 5′ATTGAGTTGGTGCCTGAAC3′ | 5′TATATCAACTGGTACAGGAAGACC3′ |
| | (SEQ ID NO: 23) | (SEQ ID NO: 24) |
| HuTRDV3 | 5′TGTGACAAAGTAACCCAGAGTTC3′ | 5′GGTACTGCTCTGCACTTACGAC3′ |
| | (SEQ ID NO: 25) | (SEQ ID NO: 26) |
| HuTRDV4/ | 5′CAAACCCAACCAGGAATG3′ | 5′AGGAAAAGGAGGCTGTGAC3′ |
| TRAV14 | (SEQ ID NO: 27) | (SEQ ID NO: 28) |
| HuTRDV5/ | 5′GCAAGTTAAGCAAAATTCACC3′ | 5′CTGCTGAAGGTCCTACATTC3′ |
| TRAV29 | (SEQ ID NO: 29) | (SEQ ID NO: 30) |
| HuTRDV6/ | 5′TTGATAGTCCAGAAAGGAGG3′ | 5′CGTTTGACTACTTTCCATGG3′ |
| TRAV23 | (SEQ ID NO: 31) | (SEQ ID NO: 32) |
| HuTRDV7/ | 5′GACAAGGTGGTACAAAGCC3′ | 5′ATCTCTGGTTGTCCACGAG3′ |
| TRAV36 | (SEQ ID NO: 33) | (SEQ ID NO: 34) |
| HuTRDV8/ | 5′CAGTCACTCAGTCTCAACCAG3′ | 5′TCTGGTACAAGCAGCCTC3′ |
| TRAV38-2 | (SEQ ID NO: 35) | (SEQ ID NO: 36) |
| HuTRDC | 5′CTTCATATTTACCAAGCTTGACAG3′ | 5′GATGACAATAGCAGGATCAAAC3′ |
| | (SEQ ID NO: 37) | (SEQ ID NO: 38) |

Nine (9) TRGV external sense primers, nine (9) TRGV internal sense primers, eight (8) TRDV external sense primers and eight (8) TRDV internal sense primers targeted for individual TRGV and TRDV families were designed based on the sequences derived from the IMGT database⁶². For the antisense primer, single TRGC external, TRGC internal, TRDC external, and TRDC internal primers complementary to the published TRGC and TRDC sequences in IMGT were designed. Human TRAV14/DV4, TRAV23/DV6, TRAV29/DV5, TRAV36/DV7, and TRAV38-2/DV8 are shared primers in TRAV and TRDV primer sets.

The primers were synthesized by IDT and stored at −20° C. at a stock concentration of 100 μM in TE with low EDTA (pH8.0). The primers for each category (sense external, sense internal of TRGV and TRDV) were combined so that the final concentration of each primer in the mixture was 10 μM. The antisense primers were diluted to 10 μM. The PCR conditions for the TCRγδ nested PCR were 95° C. for 2 min, followed by 35 cycles of 95° C. for 20 s, 53° C. for 20 s, and 72° C. for 45 s, followed by final extension of 72° C. for 7 min. The PCR products were run on a 2% agarose gel to check for the success rate of the PCR as well as contamination following which the products were purified by a modified Exonuclease I-rShrimp alkaline phosphatase (ExoSAP-IT®) method⁶³ to eliminate unincorporated primers and dNTPs for high quality DNA sequencing. 1 μl of the single cell PCR product was added into the mixture of 4.6 μl of Tris-Cl (50 mM, pH8.0), 0.2 μl of Exonuclease I and 0.2 μl of rShimp alkaline phosphatase and was incubated at 37° C. for 15 min and 80° C. for 15 min. The purified PCR products were sequenced using the relevant TRAC, TRBC, TRGC or TRDC primer. A schematic of the PCR strategy is shown in FIG. 1A.

gBlock® gene fragments, Gibson Assembly® and Transformation. The gBlock gene fragments encoding the library of TRGVs and TRDVs were obtained from Integrated DNA Technologies (IDT). The expression vector pMICherry (10 μg), which was modified from the parental pMIGII⁶⁴ by changing GFP to an mCherry reporter, was double digested by EcoR I (20 units) and Xho I (20 units) restriction enzymes (New England Biolabs) at 37° C. for 3 h as per manufacturer's instruction. Agarose gel purified-linearized pMICherry vector (100 ng) and 2× TCR gBlock inserts were ligated in a three-way ligation, including the TCRγ gene, TCRδ gene, and linearized vector by using the Gibson Assembly Cloning kit (New England Biolabs) per manufacturer's instructions. Two microliters of the ligation mixture was transformed into DH5α Competent E. coli (New England Biolabs) per manufacturer's instructions.

Generation of human CD3 constructs. Human CD3 δ, γ, ζ and (genes were amplified from human PBMC cDNA using the primers in Table 2a. All the genes were linked together by overlap PCR with species-specific 2A regions inserted³⁵. The types and amino acid sequences of the 2As used are shown in Table 2b. The CD3 gene complex was then cloned into an MSCV-based retroviral vector that contains an IRES^{31,32,65} and ametrine as a reporter gene.

TABLE 2a
2A Primers targeting human
CD3δ, γ, ε and ζ genes

| TRGV gene(s) | |
|---|---|
| targeted | |
| by primer | Primer sequence |
| CD3δ | 5′CCCTCACTCCTTCTCTAGGCGCCGGAAT |
| sense | TCGCCAGGATGGAACATAGCACG3′ |
| | (SEQ ID NO: 39) |
| CD3δ | 5′CCACGTCTCCCGCCAACTTGAGAAGGTC |
| antisense | AAAATTCAAAGTCTGTTTCACCGGTCCCTT |
| | GTTCCGAGCC3′ (SEQ ID NO: 40) |
| CD3γ | 5′GAATTTTGACCTTCTCAAGTTGGCGGGA |
| sense | GACGTGGAGTCCAACCCAGGGCCCATGGAA |
| | CAGGGGAAG3′ (SEQ ID NO: 41) |
| CD3γ | 5′CCTCGACGTCACCGCATGTTAGCAGACT |
| antisense | TCCTCTGCCCTCAGATCTTCTATTCCTCCT |
| | CAAC3′ (SEQ ID NO: 42) |
| CD3ε | 5′CAGAGGAAGTCTGCTAACATGCGGTGAC |
| sense | GTCGAGGAGAATCCTGGCCCAATGCAGTCG |
| | GGCACTC3′ (SEQ ID NO: 43) |
| CD3ε | 5′GTTTTCTTCCACGTCTCCTGCTTGCTTT |
| antisense | AACAGAGAGAAGTTCGTGGCGGATCCTCCG |
| | ATGCGTCTCTG3′ (SEQ ID NO: 44) |
| CD3ζ | 5′CTCTCTGTTAAAGCAAGCAGGAGACGTG |
| sense | GAAGAAAACCCCGGTCCCATGAAGTGGAAA |
| | GTG3′ (SEQ ID NO: 45) |
| CD3ζ | 5′GAGGGAGAGGGGCGGAATTGATCCTCGA |
| antisense | GCAATTGTTAGCGAGGGGCCAG3′ |
| | (SEQ ID NO: 46) |

TABLE 2b
Types and sequences of 2A regions

| | 2A amino acid | |
|---|---|---|
| 2A Type | sequence | Separation |
| F2A (foot- | VKQTLNFDLLKLAGD | CD3δ and |
| and-mouth | VESNPGP | CD3γ |
| disease virus) | (SEQ ID NO: 47) | |
| T2A (Thosea | EGRGSLLTCGDVEEN | CD3γ and |
| asigna virus) | PGP | CD3ε |
| | (SEQ ID NO: 48) | |
| P2A (porcine | ATNFSLLKQAGDVEE | CD3ε and |
| teschovirus-1) | NPGP | CD3ζ |
| | (SEQ ID NO: 49) | |

DNA Isolation, Cell Culture and Transfection. Recombinant pMICherry plasmids with full length TCRαβ or TCRγδ inserts were isolated in small scale by using a NucleoSpin® Plasmid kit (Clontech) and in large scale for transfection using a Plasmid Midi kit (Qiagen) per the manufacturer's instructions. The Neon® Transfection System was used to transfect 10 μg TCRαβ or γδ DNA in the pMICherry vector into the human Jurkat 76 TCRα⁻β⁻ cell line (2×10⁷ cells/mL, 100 μl)⁶⁶, followed by three pulses with a voltage of 1,350V and a width of 10 ms. The transfected cells were cultured for 48 h before being assayed for TCRαβ or TCRγδ expression on the surface by FACS analysis. The human Jurkat 76 cells TCRα⁻β⁻ cell line was cultured in complete-RPMI 1640 medium, which is RPMI 1640 with 10% of fetal bovine serum, 1% Penicillin Streptomycin, and 1% L-glutamine at 37° C. and 5% CO₂.

Immunofluorescent and flow cytometric analysis For surface staining, cells (1-5×10⁵) were harvested from culture and washed with FACS buffer (PBS with 1% of BSA and 0.1% sodium azide) prior to staining. The cells were treated with human FcR blocking reagent (Miltenyi Biotec) on ice for 20 min, and cells were then treated with various fluorescent conjugated antibodies against cell surface markers in FACS buffer. Human γδ T cells were stained with APC-conjugated anti-human TCRγ/δ (Biolegend, clone: B1) or APC-conjugated anti-human TCRα/β (Biolegend, clone: IP26) and Pacific Blue-conjugated anti-human CD3 (Biolegend, clone: OKT3). For influenza-specific tetramer staining, cells (1-5×10⁵) were stained with APC-conjugated Influenza-M1 tetramer (Beckman Coulter, HLA-A*0201, GILGFVFTL (SEQ ID NO: 146)) in FACS buffer at room temperature for 1 h prior to surface staining with the same staining antibodies described.

Modification of the CDR3 region by two-step overlap extension PCR cloning The substitution of the CDR3 was carried out by an overlap extension PCR cloning protocol²⁸. A schematic diagram of the procedure is shown in FIG. 4. Briefly, a library of linker DNA was generated by gBlock synthesis at IDT (Table 3). The linker DNA consists of TRGC-2A-TRDVx (X represents the TRDV family) sequence. Using the single cell PCR products of γ and δ chains of the desired clonotype and the relevant linker gBock DNA we carried out an overlap PCR. The PCR reaction was set up and carried out as follows: 12.5 μl 2× Phusion® high-fidelity DNA polymerase (New England Biolabs), 0.25 μl of 100×DMSO, 1 μl of 10 μM TRGV internal sense primer, 1 μl of TRDV internal antisense primer (Table 1), Ing of linker DNA, and deionized H₂O up to 25 μl. The PCR program was 98° C. for 30 s; 34 cycles of each at 98° C. for 10 s, 58° C. for 30 s, 72° C. for 1 min; then finally 72° C. for 10 min. The PCR products were visualized on a 1% agarose gel, and purified from the gel to use for cloning into the existing construct with the same TRGV and TRDV family usage. The reaction conditions used were as follows: 20 ng of a TCR construct in pMICherry vector with identical TRGV and TRDV but an irrelevant CDR3γ and 8, with 50 ng of the first step PCR products, 12.5 μl of 2× Phusion high-fidelity DNA polymerase, 0.25 μl of 100×DMSO, and deionized H₂O up to 25 μl. The PCR conditions used were 98° C. for 30 s; 17 cycles of each at 98° C. for 10 s, 65° C. for 30 s, 72° C. for 4 min; then finally 72° C. for 10 min. The PCR products were incubated with 1 μl DpnI enzyme (New England Biolabs) at 37° C. for 1 h, and 2-3 μl of the digested products transformed into NovaBlue Singles® competent cells (EMD Millipore).

TABLE 3
Human TCRγδ Linker DNA library
(lower case: TRGC sequence;
bold: 2A sequence; italic: TRDVx sequence

| Linker | |
|---|---|
| DNA Name | Sequence |
| HuLinkerDV1 | 5′catacctttgtcttcttgagaaatttttcccagata |
| | ttattaagatacattggcaagaaaagaagagcaacacg |
| | attctgggatcccaggaggggaacaccatgaagactaa |
| | cgacacatacatgaaatttagctggttaacggtgccag |
| | aagagtcactggacaaagaacacagatgtatcgtcaga |
| | catgagaataataaaaacggaattgatcaagaaattat |
| | ctttcctccaataaagacagatgtcatcacaatggatc |
| | ccaaagacaattggtcaaaagatgcaaatgatacacta |
| | ctgctgcagctcacaaacacctctgcatattacatgta |
| | cctcctcctgctcctcaagagtgtggtctattttgcca |
| | tcatcacctgctgtctgcttggaagaacggctttctgc |
| | tgcaatggagagaaatcaGCCACGAACTTCTCTCTGTT |
| | AAAGCAAGCAGGAGACGTGGAAGAAAACCCCGGTCCCA |
| | TGCTGTTCTCCAGCCTGCTGTGTGTATTTGTGGCCTTC |
| | AGCTACTCTGGATCAAGTGTGGCCCAGAAGGTTACTCA |
| | AGCCCAGTCATCAGTATCCATGCCAGTGAGGAAAGCAG |
| | TCACCCTGAACTGCCTGTATGAAACAAGTTGGTGGTCA |
| | TATTATATTTTTTGGTACAAGCAACTTCCCAGCAAAGA |
| | GATGATTTTCCTTATTCGCC3′ (SEQ ID NO: 50) |
| HuLinkerDV2 | 5′catacattgtatatgagaaatttttcccagatatta |
| | ttaagatacattggcaagaaaagaagagcaacacgatt |
| | ctgggatcccaggaggggaacaccatgaagactaacga |
| | cacatacatgaaatttagctggttaacggtgccagaag |
| | agtcactggacaaagaacacagatgtatcgtcagacat |
| | gagaataataaaaacggaattgatcaagaaattatctt |
| | tcctccaataaagacagatgtcatcacaatggatccca |
| | aagacaattggtcaaaagatgcaaatgatacactactg |
| | ctgcagctcacaaacacctctgcatattacatgtacct |
| | cctcctgctcctcaagagtgtggtctattttgccatca |
| | tcacctgctgtctgcttggaagaacggctttctgctgc |
| | aatggagagaaatcaGCCACGAACTTCTCTCTGTTAAA |
| | GCAAGCAGGAGACGTGGAAGAAAACCCCGGTCCCATGC |
| | AGAGGATCTCCTCCCTCATCCATCTCTCTCTCTTCTGG |
| | GCAGGAGTCATGTCAGCCATTGAGTTGGTGCCTGAACA |
| | CCAAACAGTGCCTGTGTCAATAGGGGTCCCTGCCACCC |
| | TCAGGTGCTCCATGAAAGGAGAAGCGATCGGTAACTAC |
| | TATATCAACTGGTACAGGAAGACCCAAGG3′ |
| | (SEQ ID NO: 51) |
| HuLinkerDV3 | 5′catacctttgtcttcttgagaaatttttcccagata |
| | ttattaagatacattggcaagaaaagaagagcaacacg |
| | attctgggatcccaggaggggaacaccatgaagactaa |
| | cgacacatacatgaaatttagctggttaacggtgccag |
| | aagagtcactggacaaagaacacagatgtatcgtcaga |
| | catgagaataataaaaacggaattgatcaagaaattat |
| | ctttcctccaataaagacagatgtcatcacaatggatc |
| | ccaaagacaattggtcaaaagatgcaaatgatacacta |
| | ctgctgcagctcacaaacacctctgcatattacatgta |
| | cctcctcctgctcctcaagagtgtggtctattttgcca |
| | tcatcacctgctgtctgcttggaagaacggctttctgc |
| | tgcaatggagagaaatcaGCCACGAACTTCTCTCTGTT |
| | AAAGCAAGCAGGAGACGTGGAAGAAAACCCCGGTCCCA |
| | TGATTCTTACTGTGGGCTTTAGCTTTTTGTTTTTCTAC |
| | AGGGGCACGCTGTGTGACAAAGTAACCCAGAGTTCCCC |
| | GGACCAGACGGTGGCGAGTGGCAGTGAGGTGGTACTGC |
| | TCTGCACTTACGACACTG3′ (SEQ ID NO: 52) |
| HuLinkerDV4 | 5′catacctttgtcttcttgagaaatttttcccagata |
| | ttattaagatacattggcaagaaaagaagagcaacacg |
| | attctgggatcccaggaggggaacaccatgaagactaa |
| | cgacacatacatgaaatttagctggttaacggtgccag |
| | aagagtcactggacaaagaacacagatgtatcgtcaga |
| | catgagaataataaaaacggaattgatcaagaaattat |
| | ctttcctccaataaagacagatgtcatcacaatggatc |
| | ccaaagacaattggtcaaaagatgcaaatgatacacta |
| | ctgctgcagctcacaaacacctctgcatattacatgta |
| | cctcctcctgctcctcaagagtgtggtctattttgcca |
| | tcatcacctgctgtctgcttggaagaacggctttctgc |
| | tgcaatggagagaaatcaGCCACGAACTTCTCTCTGTT |
| | AAAGCAAGCAGGAGACGTGGAAGAAAACCCCGGTCCCA |
| | TGTCACTTTCTAGCCTGCTGAAGGTGGTCACAGCTTCA |
| | CTGTGGCTAGGACCTGGCATTGCCCAGAAGATAACTCA |
| | AACCCAACCAGGAATGTTCGTGCAGGAAAAGGAGGCTG |
| | TGACTCTGG3′ (SEQ ID NO: 53) |
| HuLinkerDV5 | 5′catacctttgtcttcttgagaaatttttcccagata |
| | ttattaagatacattggcaagaaaagaagagcaacacg |
| | attctgggatcccaggaggggaacaccatgaagactaa |
| | cgacacatacatgaaatttagctggttaacggtgccag |
| | aagagtcactggacaaagaacacagatgtatcgtcaga |
| | catgagaataataaaaacggaattgatcaagaaattat |
| | ctttcctccaataaagacagatgtcatcacaatggatc |
| | ccaaagacaattggtcaaaagatgcaaatgatacacta |
| | ctgctgcagctcacaaacacctctgcatattacatgta |
| | cctcctcctgctcctcaagagtgtggtctattttgcca |
| | tcatcacctgctgtctgcttggaagaacggctttctgc |
| | tgcaatggagagaaatcaGCCACGAACTTCTCTCTGTT |
| | AAAGCAAGCAGGAGACGTGGAAGAAAACCCCGGTCCCA |
| | TGGCCATGCTCCTGGGGGCATCAGTGCTGATTCTGTGG |
| | CTTCAGCCAGACTGGGTAAACAGTCAACAGAAGAATGA |
| | TGACCAGCAAGTTAAGCAAAATTCACCATCCCTGAGCG |
| | TCCAGGAAGGAAGAATTTCTATTCTGAACTGTGACTAT |
| | ACTAACAGCATGTTTGATTATTTCCTATGGTACAAAAA |
| | ATACCCTGCTGAAGGTCCTACATTCCTGATATC3′ |
| | (SEQ ID NO: 54) |
| HuLinkerDV6 | 5′catacctttgtcttcttgagaaatttttcccagata |
| | ttattaagatacattggcaagaaaagaagagcaacacg |
| | attctgggatcccaggaggggaacaccatgaagactaa |
| | cgacacatacatgaaatttagctggttaacggtgccag |
| | aagagtcactggacaaagaacacagatgtatcgtcaga |
| | catgagaataataaaaacggaattgatcaagaaattat |
| | ctttcctccaataaagacagatgtcatcacaatggatc |
| | ccaaagacaattggtcaaaagatgcaaatgatacacta |
| | ctgctgcagctcacaaacacctctgcatattacatgta |
| | cctcctcctgctcctcaagagtgtggtctattttgcca |
| | tcatcacctgctgtctgcttggaagaacggctttctgc |
| | tgcaatggagagaaatcaGCCACGAACTTCTCTCTGTT |
| | AAAGCAAGCAGGAGACGTGGAAGAAAACCCCGGTCCCA |
| | TGGACAAGATCTTAGGAGCATCATTTTTAGTTCTGTGG |
| | CTTCAACTATGCTGGGTGAGTGGCCAACAGAAGGAGAA |
| | AAGTGACCAGCAGCAGGTGAAACAAAGTCCTCAATCTT |
| | TGATAGTCCAGAAAGGAGGGATTTCAATTATAAACTGT |
| | GCTTATGAGAACACTGCGTTTGACTACTTTCCATGGTA |
| | CC3′ (SEQ ID NO: 55) |
| HuLinkerDV7 | 5′catacctttgtcttcttgagaaatttttcccagata |
| | ttattaagatacattggcaagaaaagaagagcaacacg |
| | attctgggatcccaggaggggaacaccatgaagactaa |
| | cgacacatacatgaaatttagctggttaacggtgccag |
| | aagagtcactggacaaagaacacagatgtatcgtcaga |
| | catgagaataataaaaacggaattgatcaagaaattat |
| | ctttcctccaataaagacagatgtcatcacaatggatc |
| | ccaaagacaattggtcaaaagatgcaaatgatacacta |
| | ctgctgcagctcacaaacacctctgcatattacatgta |
| | cctcctcctgctcctcaagagtgtggtctattttgcca |
| | tcatcacctgctgtctgcttggaagaacggctttctgc |
| | tgcaatggagagaaatcaGCCACGAACTTCTCTCTGTT |
| | AAAGCAAGCAGGAGACGTGGAAGAAAACCCCGGTCCCA |
| | TGATGAAGTGTCCACAGGCTTTACTAGCTATCTTTTGG |
| | CTTCTACTGAGCTGGGTGAGCAGTGAAGACAAGGTGGT |
| | ACAAAGCCCTCTATCTCTGGTTGTCCACGAGGGAG3′ |
| | (SEQ ID NO: 56) |
| HuLinkerDV8 | 5′catacattgtatatgagaaatttttcccagatatta |
| | ttaagatacattggcaagaaaagaagagcaacacgatt |
| | ctgggatcccaggaggggaacaccatgaagactaacga |
| | cacatacatgaaatttagctggttaacggtgccagaag |
| | agtcactggacaaagaacacagatgtatcgtcagacat |
| | gagaataataaaaacggaattgatcaagaaattatctt |
| | tcctccaataaagacagatgtcatcacaatggatccca |
| | aagacaattggtcaaaagatgcaaatgatacactactg |
| | ctgcagctcacaaacacctctgcatattacatgtacct |
| | cctcctgctcctcaagagtgtggtctattttgccatca |
| | tcacctgctgtctgcttggaagaacggctttctgctgc |
| | aatggagagaaatcaGCCACGAACTTCTCTCTGTTAAA |
| | GCAAGCAGGAGACGTGGAAGAAAACCCCGGTCCCATGG |
| | CATGCCCTGGCTTCCTGTGGGCACTTGTGATCTCCACC |
| | TGTCTTGAATTTAGCATGGCTCAGACAGTCACTCAGTC |
| | TCAACCAGAGATGTCTGTGCAGGAGGCAGAGACCGTGA |
| | CCCTGAGCTGCACATATGACACCAGTGAGAGTGATTAT |
| | TATTTATTCTGGTACAAGCAGCCTCCCAG3′ |
| | (SEQ ID NO: 57) |

Nur77-GFP Jurkat 76 TCRα⁻β⁻ cell line. To characterize the functionality of TCRαβ or γδ clones, we established the Nur77-GFP Jurkat 76 TCRα⁻β⁻ cell line (NJ76 cells). After linearization of a Nur77-GFP BAC clone (constructed based on pTARBAC)⁴⁰ by mixing 10 μg BAC DNA, 2 μl 10× reaction buffer, 10 units of PI-SceI restriction enzyme (New England Biolabs), and nuclease-free water to make the volume up to 20 μl with incubation at 37° C. for 3 h and inactivation at 65° C. for 20 min, 80 μl of nuclease-free water, 15 μl of sterile sodium acetate (3M, pH7.0), and 300 μl of ethanol were added to the reaction mixture, which was then centrifuged at 12,000 g for 30 min at 4° C. The resulting DNA pellet was washed with 75% ethanol, dried in the air, and resuspended by Tris-EDTA buffer (pH 8.0). The Neon® Transfection System following the manufacturer's instruction was used to transfect the linearized BAC DNA into the human Jurkat 76 TCRα⁻β⁻ cell line (2×10⁷ cells/mL, 100 μl), and pulsed three times with a voltage of 1,350V and a width of 10 ms. Cells were then cultured in complete-RPMI 1640 medium containing 500 μg/ml Geneticin (Invitrogen) for selection.

Stimulation of K^{b}PB1₇₀₃⁺ TCRαβ⁺ NJ76 cells (PB1-NJ76) by flu peptide PB1. NJ76 cell transfected with a murine K^{b}PB1₇₀₃-specific TCRαβ derived from influenza-infected mice and transfected cells were incubated with mouse splenocytes (cell number ratio of PB1-NJ76/splenocytes is 2:1), the influenza PB1₇₀₃₋₇₁₁ peptide (1 μM/ml), mouse splenocytes and peptide, and mouse α-CD3 (2C11; 10 μg/ml) and human α-CD28 (CD28.2; 10 μg/ml) in c-RPMI 1640 medium at 37° C. for 4 h. The GFP expression in the mouse TCRαβ⁺ CD3⁺ cell population was quantified by flow cytometry.

Stimulation of TRGV9 TRDV2-NJ76 cells by Zoledronic acid. NJ76 cells transfected with a TRGV9/TRDV2 clone were incubated with 50 μg/ml zoledronic acid (Zometa, Novartis) in c-RPMI 1640 medium at 37° C. for 3 h, washed three times and incubated for 12 h. The GFP expression in the TCRγδ⁺ CD3⁺ cell population was quantified by flow cytometry.

### Example 2

### Paired TCRγδ Analysis of Human PBMC Samples at the Single Cell Level

A strategy was developed to characterize the paired TCRγδ repertoire in humans. The primers were designed for all non-pseudogene TRGV and TRDV regions along with antisense primers for their respective constant regions. Two sets of primers (external and internal) were designed in order to perform a nested PCR (Table 1). The PCR products were examined by agarose gel electrophoresis before sequencing (FIG. 1B). The average success rate for obtaining paired CDR3γ and CDR3δ sequences at the single cell level from human PBMC samples by the present method is 71,25±18.75%. The TRGV/TRDV family usage was determined from the multiplex PCR products (FIG. 1C). On average, 20% of the sequences from the analysis of 14 human PBMCs were TRGV9/TRDV2. This technique along with the established mouse and human aß single cell multiplex PCR offers a rapid method (turnaround time ˜3 days per 160 cells) for characterizing paired TCRγδ chains at the single cell level. The data of paired TRGV/TRDV family usage percentage in each human sample are shown in Table 4.

Table 4: TRGV/TRDV repertoire among 14 human samples. The percentage of paired TRGV/TRDV usage was analyzed from the sequencing results of each 14 human PBMC samples. Average values, standard deviation and standard error were reported.

TABLE 4

| — | Mean | Std. Deviation | Std. Error |
|---|---|---|---|
| TRGV9/TRDV2 | 20.06 | 16.11 | 4.306 |
| TRGV4/TRDV1 | 12.71 | 15 | 4.008 |
| TRGV8/TRDV1 | 8.714 | 10.63 | 2.842 |
| TRGV9/TRDV3 | 8.357 | 21.25 | 5.68 |
| TRGV2/TRDV1 | 7.836 | 14.61 | 3.906 |
| TRGV1/TRDV2 | 4.643 | 13.51 | 3.61 |
| TRGV3/TRDV3 | 4.35 | 13.09 | 3.498 |
| TRGV9/TRDV1 | 4.279 | 8.702 | 2.326 |
| TRGV4/TRDV3 | 4.071 | 9.059 | 2.421 |
| TRGV3/TRDV1 | 4.029 | 5.646 | 1.509 |
| TRGV2/TRDV3 | 3.071 | 8.801 | 2.352 |
| TRGV5/TRDV3 | 2.643 | 6.744 | 1.802 |
| TRGV8/TRDV2 | 1.971 | 5.388 | 1.44 |
| TRGV5/TRDV1 | 1.871 | 3.633 | 0.9711 |
| TRGV2/TRDV2 | 1.871 | 3.633 | 0.9711 |
| TRGV8/TRDV3 | 1.643 | 4.557 | 1.218 |
| TRGV2/TRAV29/DV5 | 1.492 | 4.706 | 1.305 |
| TRGV3/TRDV2 | 1.279 | 2.922 | 0.7808 |
| TRGV10/TRDV1 | 1.071 | 2.31 | 0.6175 |
| TRGV9/TRAV38-2/DV8 | 0.3571 | 1.336 | 0.3571 |
| TRGV4/TRDV2 | 0.1857 | 0.6949 | 0.1857 |
| TRGV9/TRAV29/DV5 | 0.1714 | 0.6414 | 0.1714 |

### Example 3

### Establishment of Human TCRαβ and TCRγδ Retroviral Expression Clone Library

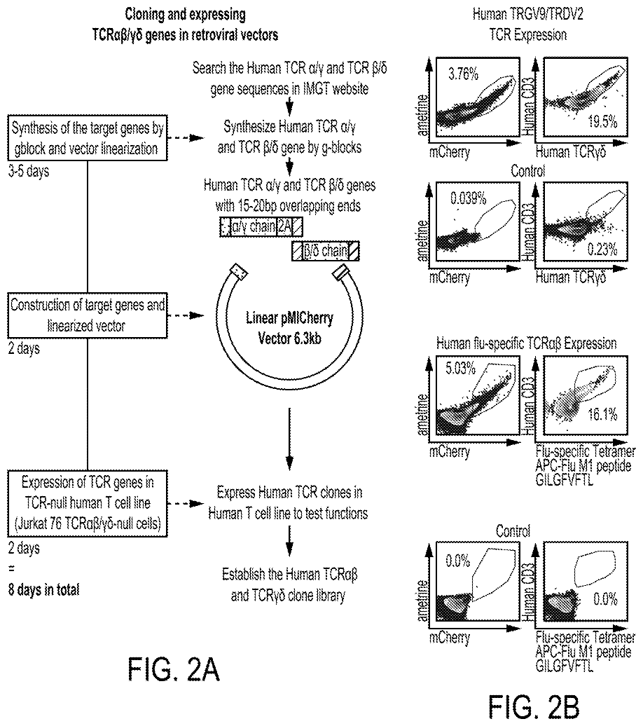

Many of the downstream applications of paired TCRαβ or TCRγδ sequence analysis require cloning and expression of the antigen specific receptors for immunological studies such as structural and functional characterization, biochemical characterization, epitope identification, and gene therapy^{18,29,30}). Thus, a rapid cloning method was developed to improve on conventional restriction enzyme-mediated ligation techniques, which can be cumbersome and time consuming. In addition, use of restriction enzymes for cloning becomes problematic because of the potential occurrences of restriction sites in some variable regions and the non-germline CDR3 sequences of the TCR chains. The vector used for TCR expression is pMICherry, which has been successfully used to construct TCR clones for the generation of retrogenic mice^{31,32}. A schematic diagram of the cloned TCR chains in the pMICherry vector is shown in FIG. 2A.

To clone full length TCR chains, a TRGV9/TRDV2 clone was chosen to demonstrate the feasibility of the cloning system, since the TRGV9/TRDV2 clonotype is dominant in the TCRγδ repertoire analysis from human PBMCs (FIG. 1C). Similarly, a human TCRαβ pair was chosen derived from an influenza-specific CD8 T cell from an infected individual. Using the IMGT-reported human TRGV, TRDV, TRGC, and TRDC sequences for TCRγδ or human TRAV, TRBV, TRAC and TRBC sequences for TCRαβ and the single cell CDR3γ and 8 or CDR3α and β sequence data full length TCRγ and 8 chains and TCRα and β chains were constructed and joined by the 2A “self-cleaving” site in silico. 2A oligopeptides can interact with the ribosomal exit tunnel to terminate sequence translation at the final codon (Pro) of the 2A sequence, and reinitiate translation of the following sequence 3.3. Multi-cistronic 2A based retroviral vectors have been used for TCR:CD3 structural and functional studies^{32,34,38}. The entire sequence of TCRγ-2A-TCRδ along with an 25 bp overhang complementary to the ends of the linearized pMICherry vector were synthesized in two fragments of approximately 1 kb each as gBlockR DNA fragments (IDT) with an internal 25 bp overlap in the 2A segment. By using Gibson AssemblyR Master Mix, the two gBlocks spanning the TCRγ-2A-TCRδ were ligated with the linearized vector in a three-way ligation. The process of cloning is shown in FIG. 2A. After this cloning procedure, an average of 70.9% of the colonies picked after transformation was entirely matched with target sequences. The others contained either point mutations resulting from the cloning process or no inserts. More than 30 different TCR constructs have been cloned by using this system, including, mouse and human TCR αβ and TCRγδ. The cloning system is highly reproducible.

To test the functionality of the TCR clones that were made following the method described in FIG. 2A, the human TRGV9/TRDV2 construct was transfected into the Jurkat 76 TCRα⁻β⁻ cell line and checked for the cell surface expression by anti-TCRγδ and anti-CD3 antibody staining and flow cytometry. Although Jurkat cells have endogenous CD3, the expression of TCRγδ was not robust. Since γδ T cells do not develop in CD3-deficient mice and patients³⁹ the human CD3 complex was cloned into an MSCV vector (pMIAmetrine) and co-transfected it with the human TCR constructs. mCherry and ametrine are the reporter genes in the pMICherry vector with human TCR genes and the pMIAmetrine vector with human CD3 genes, respectively. It is demonstrated that co-transfection of the human CD3 construct with the human TCRγδ and αβ chains can improve the surface expression level of TCR in a Jurkat cell line (FIG. 6). 3.76% of cells were double positive for mCherry and ametrine, and 19.5% of double-positive cells were TCRγδ and CD3 positive, which proved the functionality of the cloning and expression platform of the invention (top panel, FIG. 2B). The expression of the influenza virus-specific TCRαβ was analyzed by staining the transfected cells with APC-conjugated influenza-M1 tetramer (HLA-A*0201, GILGFVFTL (SEQ ID NO: 146)) and CD3 antibody. The FACS plot shows 5.03% of transfected cells were double positive for mCherry and ametrine, 16.1% of which were positive for tetramer staining (bottom panel, FIG. 2B).

### Example 4

Effective TCR Activation Reporting by Nur77-GFP Jurkat 76 TCRα⁻β⁻ Cells

An important application of TCR cloning and expression is to screen molecules that can activate or inhibit TCR signaling. Thus far, the common methods to detect TCR activation are using ELISA to detect cytokines (e.g. IFNγ) in the cell culture medium^{21,22}, intracellular staining to report cytokine production by flow cytometry, or qRT-PCR to quantify the mRNA expression of cytokines, which are time-consuming, labor-intensive, and expensive. In the present invention, a TCR activation reporter cell line, Nur77-GFP Jurkat 76 TCRα⁻β⁻ (NJ76 cells) was established by stably transducing Nur77-GFP BAC DNA into Jurkat 76 TCRα⁻β⁻ cells. The Nur77-GFP reporting system has been demonstrated to reflect specific TCR signal strength by GFP expression^{40,41,42}.

To test the functionality of NJ76 cells in reporting TCR activation, NJ76 cells were transfected with a murine K^{b}PB1₇₀₃-specific TCR αβ derived from influenza-infected mice along with a mouse CD3 construct. The K^{b}PB1₇₀₃⁺TCRαβ⁺ NJ76 cells (PB1-NJ76) were incubated with mouse splenocytes, the influenza PB1₇₀₃ peptide, splenotytes and peptide, or mouse α-CD3/human α-CD28 as a positive control for 4 hours and GFP expression in transfected NJ76 cells was detected by flow cytometry (FIG. 3A). The quantification of GFP levels is shown in FIG. 3B. The results show that PB1-NJ76 cells can robustly express GFP after specific peptide stimulation (PB1) with antigen-presenting cells. The gating strategy for GFP detection is shown in FIG. 7.

This TCR-activation reporting system has also been tested for TCRγδ signaling. Zoledronic acid (Zometa, Novartis) is an aminobisphosphonate that has demonstrated antitumor effects via inhibition of tumor growth and angiogenesis and induction of malignant cell apoptosis in humans^{43,44,45}. In addition, zoledronic acid can specifically stimulate and expand human TRGV9/TRDV2 cells^{46,47,48,49}. Since it can result in the accumulation of upstream metabolites in the mevalonate pathway, e.g. IPP, which induce the expansion of γδ T cells in vitro and in vivo, zoledronic acid pre-treatment can increase the cytolysis of some cancer cell lines by γδ T cells⁵⁰. After transfection of the human TRGV9/TRDV2 vector and human CD3 vector into NJ76 cells, the transfected TRGV9/TRDV2-NJ76 cells were pulsed with 50 μg/ml of zoledronic acid for 3 hours, and washed and incubated the cells at 37° C. for 12 h. The GFP expression level was quantified in transfected TRGV9/TRDV2-NJ76 cells and control cells by flow cytometry. The Nur77-GFP expression level is shown in FIG. 3C (top panel). The transfected TRGV9/TRDV2-NJ76 cells showed a significantly higher level of GFP expression, which demonstrates that zoledronic acid can trigger γδ TCR signaling for the stimulation and expansion of γδ T cells. The fold change of GFP expression over time in stimulated TRGV9/TRDV2-NJ76 cells with zoledronic acid and non-stimulated TRGV9/TRDV2-NJ76 is shown in FIG. 3C (bottom panel).

### Example 5

Effective TCR Activation Reporting by Nur77-Luc Jurkat 76 TCRα⁻β⁻ Cells

Nur77-GFP BAC DNA was modified by using recombineering to substitute the GFP with firefly luciferase. In particular, recombineering was undertaken to insert a cassette with Luciferase-SV40 pA-PGK-Neo-bGHpA into the Nur77 gene. A clone with such insertion (Nur77-Luc BAC DNA) was isolated and sequenced. TCRα⁻β⁻ Jurkat 76 cells were transfected with the Nur77-Luc BAC DNA under a selectively complete RPMI medium with 500 μg/ml of Genetcin, yielding Nur77-Luciferase TCRα β-Jurkat cells.

To determine optimum conditions for luciferase assays, tests of various numbers of Nur77-Luciferase TCRα⁻β⁻ Jurkat cells per well were undertaken. In the tests, the cells were subjected to four hours of PMA/lonomycin stimulation followed by measurement in a luciferase reader. These values are shown in Table 5 under the column “Stimulated”. As a control, the luciferase reader was used to measure cells that were not stimulated. See Table 5, “Unstimulated” column. By comparison, the medium itself gave values of only 0 to 80 in the luciferase reader.

TABLE 5
Luciferase assay of various amounts of Nur77-Luciferase
TCRα⁻β⁻ Jurkat cells

| Cell number | Unstimulated | Stimulated |
|---|---|---|
| 1 × 10⁴ | 2640 | 9600 |
| 2.5 × 10⁴ | 4400 | 19680 |
| 5 × 10⁴ | 8640 | 37360 |
| 1 × 10⁵ | 16400 | 62400 |
| 2.5 × 10⁵ | 34560 | 97840 |
| 5 × 10⁵ | 41520 | 49520 |
| 1 × 10⁶ | 39440 | 32880 |

When assaying luciferase, using a cell number of 1×10⁵ provides a four-fold rise in luciferase activity as compared with the unstimulated cells.

### Example 6

### Rapid TCR Cloning by CDR3 Substitution Using Overlap Extension PCR and TCR Library

For TCRs, the only hypervariable regions are the CDR3 regions. Thus, cloning full length TCRs de novo for each application may expend unnecessary resources. To improve on this, a library was generated containing potential TRGV and TRDV “backbone” combinations that only require the swapping of individual CDR3 regions directly from PCR products. For example, in TRGV9/TRDV2 cells from PBMCs of healthy donors, the CDR3s of both γ and δ chains were found to be highly diverse (Table 4). To rapidly generate an array of TRGV9/TRDV2 clones with diverse CDR3γ and δ, a DNA linker, whose ends overlap with the TRGC and TRDV2 of the single cell PCR products, was designed. Similarly for other combinations of the TRGV/TRDV family several DNA linkers were designed. These DNA linkers contain the TRGC region, 2A and one of the TRDV regions, as is shown in FIG. 4. By overlap PCR with the single cell PCR products, DNA linkers, TRGV sense primers, and TRDC antisense primers, any pair of TCRγδ can be connected together. Next, the first-step PCR products were used as a mega primer with the appropriate clone from the library (e.g., TRGV9/TRDV2 with an irrelevant CDR3) as a template for the second-step overlap extension PCR. By using this substitution method, different γδ TCRs with matched CDR3s from the human single cell PCR products were successfully cloned. The same approach could be used with αβ TCRs, although the clone library would be larger. This CDR3 substitution approach can shorten the cloning process to within 5 days (FIG. 4).

TABLE 6
CDR3 amino acid sequences of paired human
TRGV9/TRDV2 cells isolated from PBMCs (n = 14)

| Paired amino acid sequence | Paired amino acid sequence | |
|---|---|---|
| in TRGV9-CDR3 region | in TRDV2-CDR3 region | Frequency |
| ALFIQELGKKIKV (SEQ ID NO: 58) | ACDVLGDTEGRLI (SEQ ID NO: 59) | 2 |
| ALWDGPYYKKL (SEQ ID NO: 60) | ACDTVFTGGYSSWDTRQMF (SEQ ID NO: 61) | 2 |
| ALWDIPPGQELGKKIKV (SEQ ID NO: 62) | ACDTLGETSSWDTRQMF (SEQ ID NO: 63) | 2 |
| ALWEAQELGKKIKV (SEQ ID NO: 64) | ACDSGGYSSWDTRQMF (SEQ ID NO: 65) | 2 |
| ALWEARQELGKKIKV (SEQ ID NO: 66) | ACDTLFPGGSATDKLI (SEQ ID NO: 67) | 2 |
| ALWEGTRGQELGKKIKV (SEQ ID NO: 68) | ACDTVGAHTDKLI (SEQ ID NO: 69) | 2 |
| ALWEVGDQELGKKIKV (SEQ ID NO: 70) | ACDPLNTGGSFSLYTDKLI (SEQ ID NO: 71 | 2 |
| ALWEVHSELGKKIKV (SEQ ID NO: 72) | ACDTGGFRSSWDTRQMF (SEQ ID NO: 73) | 2 |
| ALWEVHSELGKKIKV (SEQ ID NO: 72) | ACDTGGFRSSWDTRQMF (SEQ ID NO: 73) | 2 |
| ALWEVLELGKKIKV (SEQ ID NO: 74) | ACDTVGMGIRLGDKLI (SEQ ID NO: 75) | 2 |
| ALWEVLVGELGKKIKV (SEQ ID NO: 76) | ACDILGINTDKLI (SEQ ID NO: 77) | 2 |
| ALWEVPELGKKIKV (SEQ ID NO: 78) | ACERLGDYVPDKLI (SEQ ID NO: 79) | 2 |
| ALWEVQELGKKIKV (SEQ ID NO: 80) | ACDRLLGDTDKLI (SEQ ID NO: 81) | 2 |
| ALWEVQELGKKIKV (SEQ ID NO: 80) | ACDTVAPRIGGLKYTDKLI (SEQ ID NO: 82) | 2 |
| ALWEVQELGKKIKV (SEQ ID NO: 80) | ACDTVGGPYTDKLI (SEQ ID NO: 83) | 2 |
| ALWEVQELGKKIKV (SEQ ID NO: 80) | ACDTVGGTAQ (SEQ ID NO: 84) | 2 |
| ALWEVQELGKKIKV (SEQ ID NO: 80) | ACDTVSGGSTPTWYTDKLI (SEQ ID NO: 85) | 2 |
| ALWEVQELGKKIKV (SEQ ID NO: 80) | ACDTVSIFTGDTTDKLI (SEQ ID NO: 86) | 2 |
| ALWEVRELGKKIKV (SEQ ID NO: 87) | ACDTILIFSPTGGDTDKLI (SEQ ID NO: 88) | 2 |
| ALWEVRELGKKIKV (SEQ ID NO: 87) | ACVPLGDWTDKLI (SEQ ID NO: 89) | 2 |
| ALWEVRKQELGKKIKV (SEQ ID NO: 90) | ACDTLGDDFDKLI (SEQ ID NO: 91) | 2 |
| ALWEVTHNRQELGKKIKV (SEQ ID NO: 92) | ACDTLLGTEAWDTRQMF (SEQ ID NO: 93) | 2 |
| ALWGGAAGAYYKKL (SEQ ID NO: 94) | ACDGKTTDTDKLI (SEQ ID NO: 95) | 2 |
| ALWGGELGKKIKV (SEQ ID NO: 96) | ACDLLGDTRYTDKLI (SEQ ID NO: 97) | 2 |
| ALWVQELGKKIKV (SEQ ID NO: 98) | ACVGITGDTDKLI (SEQ ID NO: 99) | 2 |
| ALWEAHQELGKKIKV (SEQ ID NO: 100) | ACDSLGDSVDKLI (SEQ ID NO: 101) | 1 |
| ALWEANKKL (SEQ ID NO: 102) | ACDLLRGAGGQIDKLI (SEQ ID NO: 103) | 1 |
| ALWEAQELGKKIKV (SEQ ID NO: 104) | ACDTVGGAFDTDKLI (SEQ ID NO: 105) | 1 |
| ALWEATGLGKKIKV (SEQ ID NO: 106) | ACDMGDTRSWDTRQMF (SEQ ID NO: 107) | 1 |
| ALWEDLELGKKIKV (SEQ ID NO: 108) | ACDTVSWGKNTDKLI (SEQ ID NO: 109) | 1 |
| ALWEKEELGKKIKV (SEQ ID NO: 110) | ACDTGDWGSSWDTRQMF (SEQ ID NO: 111) | 1 |
| ALWEKELGKKIKV (SEQ ID NO: 112) | ACDILDSTGGTDLTAQLF (SEQ ID NO: 113) | 1 |
| ALWEMTQELGKKIKV (SEQ ID NO: 114) | ACDTVRNTGGYAFAGIDKLI (SEQ ID NO: 115) | 1 |
| ALWEPQELGKKIKV (SEQ ID NO: 116) | ACDKVLGDSSWDTRQMF (SEQ ID NO: 117) | 1 |
| ALWESKELGKKIKV (SEQ ID NO: 118) | ACEGLGATQSSWDTRQMF (SEQ ID NO: 119) | 1 |
| ALWEVGELGKKIKV (SEQ ID NO: 120) | ACDKLLGDNELI (SEQ ID NO: 121) | 1 |
| ALWEVHKLGKKIKV (SEQ ID NO: 122) | ACDSLLGKGTDKLI (SEQ ID NO: 123) | 1 |
| ALWEVKELGKKIKV (SEQ ID NO: 124) | ACDTLRGSADKLI (SEQ ID NO: 125) | 1 |
| ALWEVLQQELGKKIKV (SEQ ID NO: 126) | ACDTVPARHTDKLI (SEQ ID NO: 127) | 1 |
| ALWEVPVLGKKIKV (SEQ ID NO: 128) | ACDTADRSSYTDKLI (SEQ ID NO: 129) | 1 |
| ALWEVQELGKKIKV (SEQ ID NO: 80) | ACDTLLGDPSSSWDTRQMF (SEQ ID NO: 130) | 1 |
| ALWEVQELGKKIKV (SEQ ID NO: 80) | ACDTLSGGYARTDKLI (SEQ ID NO: 131) | 1 |
| ALWEVQELGKKIKV (SEQ ID NO: 80) | ACDTVGILGDTGLGLI (SEQ ID NO: 132) | 1 |
| ALWEVRELGKKIKV (SEQ ID NO: 87) | ACDTIVSGYDGYDKLI (SEQ ID NO: 133) | 1 |
| ALWEVRELGKKIKV (SEQ ID NO: 87) | ACSILGDKTSDKLI (SEQ ID NO: 134) | 1 |
| ALWEVRQELGKKIKV (SEQ ID NO: 135) | ACDTVSQRGGYSDKLI (SEQ ID NO: 136) | 1 |
| ALWEVRVQELGKKIKV (SEQ ID NO: 137) | ACDPLERVGGPANTDKLI (SEQ ID NO: 138) | 1 |
| ALWEVTELGKKIKV (SEQ ID NO: 139) | ACDVLGDTGDDKLI (SEQ ID NO: 140) | 1 |
| ALWGRELGKKIKV (SEQ ID NO: 141) | ACDTVGSNTDKLI (SEQ ID NO: 142) | 1 |
| ALWVQELGKKIKV (SEQ ID NO: 98) | ACDVLGDTEADKLI (SEQ ID NO: 143) | 1 |
| ALYGSPSGEELGKKNQG (SEQ ID NO: 144) | ACDPLEGAGGHNTDKLI (SEQ ID NO: 145) | 1 |

TABLE 7
Sequences of primers to mouse
sequences used in nested RT-PCR.
(All primers are forward primers except two
sequences indicated with “reverse” which represents
reverse primers. External primers were used in
the first round while internal ones were used in the
second round of PCR.)

| Primer | | SEQ | | SEQ |
|---|---|---|---|---|
| name | External | ID | Internal | ID |
| TRGV1-3 | GCAGCTGGAGCAAACTG | 147 | CTGAATTATCGGTCACCAG | 148 |
| TRGV4 | CAAATATCCTGTAAAGT | 149 | GTTTAGAGTTTCTATTATA | 150 |
| | TTTCATC | | TGTCCTTGCAAC | |
| TRGV5 | GATATCTCAGGATCAGC | 151 | TACCCGAAGACCAAACAAG | 152 |
| | TCTCC | | AC | |
| TRGV6 | TCACCTCTGGGGTCATA | 153 | AGAGGAAAGGAAATACGGC | 154 |
| | TG | | | |
| TRGV7 | CAACTTGGAAGAAAGAA | 155 | CACCAAGCTAGAGGGGTC | 156 |
| | TAATGTC | | | |
| TRGC | CTTTTCTTTCCAATACA | 157 | TCDGGAAAGAACTTTTCAA | 158 |
| (reverse) | CCC | | GG | |
| TRDV1 | ACCCAAATGTTGCATCA | 159 | GTCTCTGACAATCCAAGAA | 160 |
| | G | | GG | |
| TRDV2 | TCTGTGCAGGTGGCAG | 161 | CCCTGGACTGCACCTATG | 162 |
| TRDV4 | TGTATATTTGGAACCAG | 163 | GATCCTGCCTCCTTCTACT | 164 |
| | TTGC | | G | |
| TRDV5 | CATCACGCTGACCCAG | 165 | GCTCCACTGACCAGACAG | 166 |
| TRDV6/ | CASCTTYTTAGTGGAGA | 167 | AYTCTGTAGTCTTCCAGAA | 168 |
| TRAV15 | GATGG | | ATCAC | |
| TRDV7/ | TCCTTGGTTCTGCAGG | 169 | TGCAGGAGGGGGAGA | 170 |
| TRAV13 | | | | |
| TRDV8/ | GCAGCAGGTGAGACAAA | 171 | CTCTGACAGTCTGGGAAGG | 172 |
| TRAV14 | G | | | |
| TRDV9/ | CAGATGCAAGGTCAAGT | 173 | GGAGAAGGTCCACAGCTC | 174 |
| TRAV6-1/ | GAC | | | |
| 6-2 | | | | |
| TRDV9/ | AAGGTCCACAGCTCCTT | 175 | CAACTGCCAACAACAAGG | 176 |
| TRAV6-3/ | C | | | |
| 6-4 | | | | |
| TRDV9/ | GTTCTGGTATGTGCAGT | 177 | TCCTTCCACTTGCAGAAAG | 178 |
| TRAV6-5/ | ATCC | | | |
| 6-7 | | | | |
| TRDV10/ | TCTGSTCTGAGATGCAA | 179 | GGITIMAGGAACAAAGGAG | 180 |
| TRAV4 | TTTT | | AAT | |
| TRDV11/ | GTACAAGCAAACAGCAA | 181 | ATTATTCTCTGAACTTTCA | 182 |
| TRAV16 | GTG | | GAAGC | |
| TRDV12/ | GTGCACTTGCCTTGTAG | 183 | AATAGTATGGCTTTCCTGG | 184 |
| TRAV21 | C | | C | |
| TRDC | TGAAAGAATTTTGCATA | 185 | GAGATGACTATAGCAGGGT | 186 |
| (reverse) | TGGTTC | | CG | |

Tables 8-11: “Partial” V and C regions amplified depending on the target sequence. (Column “Region” discloses nucleotide sequence positions based on the IMGT reference database, see Lefranc, M.-P., The Immunologist, 7, 132-136 (1999) and www.imgt.org/IMGTScientificChart/Nomenclature/IMGT-FRCDRdefinition.html.)

TABLE 8
Human γδ primer list with positions.

| | SEQ | | |
|---|---|---|---|
| | ID | | |
| Primer name | NO: | Primer Sequence | Region |
| HuTRDV4/ | 27 | CAAACCCAACCAGGA | 76 . . . 93 |
| TRAV14Ext | | ATG | |
| HuTRDV6/ | 31 | TTGATAGTCCAGAAA | 115 . . . 134 |
| TRAV2Ext | | GGAGG | |
| HuTRDV/ | 29 | GCAAGTTAAGCAAAA | 84 . . . 104 |
| TRAV29Ext | | TTCACC | |
| HuTRDV7/ | 33 | GACAAGGTGGTACAA | 67 . . . 85 |
| TRAVExt | | AGCC | |
| HuTRDV8/ | 35 | CAGTCACTCAGTCTC | 68 . . . 88 |
| TRAVExt | | AACCAG | |
| HuTRDV1Ext | 21 | GCCCAGAAGGTTACT | 61 . . . 79 |
| | | CAAG | |
| HuTRDV2Ext | 23 | ATTGAGTTGGTGCCT | 61 . . . 79 |
| | | GAAC | |
| HuTRDV3Ext | 25 | TGTGACAAAGTAACC | 52 . . . 74 |
| | | CAGAGTTC | |
| HuTRDCExt | 37 | CTTCATATTTACCAA | 196-173 |
| | | GCTTGACAG | |
| HuTRDV4/ | 28 | AGGAAAAGGAGGCTG | 101 . . . 119 |
| TRAV14Int | | TGAC | |
| HuTRDV6/ | 32 | CGTTTGACTACTTTC | 170 . . . 189 |
| TRAV2Int | | CATGG | |
| HuTRDV/ | 30 | CTGCTGAAGGTCCTA | 197 . . . 216 |
| TRAV29Int | | CATTC | |
| HuTRDV7/ | 34 | ATCTCTGGTTGTCCA | 90 . . . 108 |
| TRAVInt | | CGAG | |
| HuTRDV8/ | 36 | TCTGGTACAAGCAGC | 164 . . . 181 |
| TRAVInt | | CTC | |
| HuTRDV1Int | 22 | AGCAAAGAGATGATT | 184 . . . 205 |
| | | TTCCTTA | |
| HuTRDV2Int | 24 | TATATCAACTGGTAC | 157 . . . 180 |
| | | AGGAAGACC | |
| HuTRDV3Int | 26 | GGTACTGCTCTGCAC | 108 . . . 129 |
| | | TTACGAC | |
| HuTRDCInt | 38 | GATGACAATAGCAGG | 150-129 |
| | | ATCAAAC | |
| HuTRGV10Ext | 13 | TTATCAAAAGTGGAG | 52 . . . 72 |
| | | CAGTTC | |
| HuTRGV11Ext | 15 | GAACAACCTGAAATA | 61 . . . 84 |
| | | TCTATTTCC | |
| HuTRGV3.5Ext | 1 | TCTTCCAACTTGGAA | 55 . . . 72 |
| | | GGG | |
| HuTRGV8Ext | 9 | CCAACTTGGAAGGGA | 59 . . . 77 |
| | | GAAC | |
| HuTRGV1.2.4.6Ext | 17 | GGGTCATCTGCTGAA | 100 . . . 119 |
| | | ATCAC | |
| HuTRGV9Ext | 11 | cCAGGTCACCTAGAG | 61 . . . 79 |
| | | CAAC | |
| HuTRGVAExt | 5 | GGGTCATCCTGTTTC | 26 . . . 43 |
| | | CAG | |
| HuTRGVBExt | 7 | TGGCCTCCCAAAGTA | 18 . . . 35 |
| | | CTG | |
| HuTRGCExt | 19 | GGTGTTCCCCTCCTG | 186-171 |
| | | G | |
| HuTRGV10Int | 14 | CAGCTATCCATTTCC | 73 . . . 91 |
| | | ACGG | |
| HuTRGV11Int | 16 | CATATCTTGGAAGGC | 108 . . . 126 |
| | | ATCC | |
| HuTRGV3.5Int | 2 | GGTCATCTGCTGAAA | 101 . . . 119 |
| | | TCAC | |
| HuTRGV1.2.4.6Int | 18 | CCAGGAGGGGAAGGC | 168 . . . 182 |
| HuTRGV8Int | 10 | AAAATGCCGTCTACA | 83 . . . 100 |
| | | CCC | |
| HuTRGV9Int | 12 | TGTCCATTTCATATG | 209 . . . 227 |
| | | ACGG | |
| HuTRGCInt | 20 | CCCAGAATCGTGTTG | 167-151 |
| | | CT | |

TABLE 9
Mouse αβ primer list with positions
(V region external and C region reverse primers
and their positions).

| | SEQ | | |
|---|---|---|---|
| | ID | | |
| Primer name | NO: | Primer Sequence | Region |
| mTRBV1Ext | 187 | TACCACGTGGTCAAGCT | 101 . . . 118 |
| | | G | |
| mTRBV12Ext | 188 | GGGGTTGTCCAGTCTCC | 94 . . . 110 |
| mTRBV13Ext | 189 | GCTGCAGTCACCCAAAG | 55 . . . 71 |
| mTRBV16Ext | 190 | CCTAGgcACAAGGTGAC | 73 . . . 91 |
| | | AG | |
| mTRBV14Ext | 191 | GCAGTCCTACAGGAAGG | 88 . . . 105 |
| | | G | |
| mTRBV15Ext | 192 | GAGTTACCCAGACACCC | 65 . . . 83 |
| | | AG | |
| mTRBV17Ext | 193 | GAAGCCAAACCAAGCAC | 168 . . . 184 |
| mTRBV19Ext | 194 | GATTGGTCAGGAAGGGC | 99 . . . 115 |
| mTRAV2Ext | 195 | cATcTACTGGTACCGAC | 159 . . . 178 |
| | | AGG | |
| mTRBV2Ext | 196 | CAGTATCTAGGCCACAA | 130 . . . 149 |
| | | TGC | |
| mTRBV20Ext | 197 | GGATGGAGTGTCAAGCT | 101 . . . 118 |
| | | G | |
| mTRBV23Ext | 198 | CTGCAGTTACACAGAAG | 62 . . . 80 |
| | | CC | |
| mTRBV24Ext | 199 | CAGACTCCACGATACCT | 73 . . . 91 |
| | | GG | |
| mTRBV29Ext | 200 | GCTGGAATGTGGACAGG | 117 . . . 133 |
| mTRBV3Ext | 201 | CCCAAAGTCTTACAGAT | 61 . . . 80 |
| | | CCC | |
| mTRBV31Ext | 202 | CTAACCTCTACTGGTAC | 143 . . . 165 |
| | | TGGCAG | |
| mTRBV4Ext | 203 | GACGGCTGTTTTCCAGA | 60 . . . 77 |
| | | C | |
| mTRBV5Ext | 204 | GGTATAAACAGAGCGCT | 155 . . . 174 |
| | | GAG | |
| Cba Rev Ext | 205 | CCAGAAGGTAGCAGAGA | 252-233 |
| | | CCC | |
| mTRBV1Int | 206 | GTATCCCTGGATGAGCT | 150 . . . 167 |
| | | G | |
| mTRBV12Int | 207 | CCAGCAGATTCTCAGTC | 269 . . . 286 |
| | | C | |
| mTRBV13Int | 208 | GTACTGGTATCGGCAGG | 147 . . . 165 |
| | | AC | |
| mTRBV14Int | 209 | GGTATCAGCAGCCCAGA | 158 . . . 175 |
| | | G | |
| mTRBV15Int | 210 | GTGTGAGCCAGTTTCAG | 123 . . . 140 |
| | | G | |
| mTRBV16Int | 211 | GAAGCAACTCTGTGGTG | 109 . . . 127 |
| | | TG | |
| mTRBV17Int | 212 | GAACAGGGAAGCTGACA | 219 . . . 236 |
| | | C | |
| mTRBV19Int | 213 | GGTACCGACAGGATTCA | 167 . . . 184 |
| | | G | |
| mTRBV2Int | 214 | GGACAATCAGACTGCCT | 222 . . . 239 |
| | | C | |
| mTRBV20Int | 215 | GCTTGGTATCGTCAATC | 142 . . . 159 |
| | | G | |
| mTRBV23Int | 216 | GCCAGGAAGCAGAGATG | 104 . . . 120 |
| mTRBV26Int | 217 | GAggTGTATCCCTGAAA | 120 . . . 139 |
| | | AGG | |
| mTRBV24Int | 218 | GCACACTGCCTTTTACT | 141 . . . 159 |
| | | GG | |
| mTRBV29Int | 219 | GTACTGGTATCGACAAG | 153 . . . 173 |
| | | ACCC | |
| mTRBV3Int | 220 | GATATGGGGCAGATGGT | 97 . . . 114 |
| | | G | |
| mTRBV31Int | 221 | CTGTTGGCCAGGTAGAG | 206 . . . 224 |
| | | TC | |
| mTRBV5Int | 222 | GCCAGAGCTCATGTTTC | 180 . . . 198 |
| | | TC | |
| Cbb Rev Int | 223 | GGGTAGCCTTTTGTTTG | 88-68 |
| | | TTTG | |
| mTRAV10.10aInt | 224 | CTACACTGAGTGTTCGA | 89 . . . 109 |
| | | GAGG | |
| mTRAV12Int | 225 | GGTTCCACGCCACTC | 242 . . . 256 |
| mTRAV13Int | 170 | TGCAGGAGGGGGAGA | 98 . . . 112 |
| mTRAV14Int | 172 | CTCTGACAGTCTGGGAA | 113 . . . 131 |
| | | GG | |
| mTRAV15Int | 168 | AYTCTGTAGTCTTCCAG | 251 . . . 274 |
| | | AAATCAC | |
| mTRAV16Int | 182 | ATTATTCTCTGAACTTT | 248 . . . 271 |
| | | CAGAAGC | |
| mTRAV17Int | 226 | TATGAAGGAGCCTCCCT | 97 . . . 114 |
| | | G | |
| mTRAV18Int | 227 | CAAGATTTCACCGCACG | 103 . . . 119 |
| mTRAV19Int | 228 | GCTGACTGTTCAAGAGG | 108 . . . 126 |
| | | GA | |
| mTRAV1Int | 229 | CTCCACATTCCTGAGCC | 237 . . . 253 |
| mTRAV21Int | 184 | AATAGTATGGCTTTCCT | 220 . . . 239 |
| | | GGC | |
| mTRAV2Int | 230 | ACTCTGAGCCTGCCCT | 265 . . . 280 |
| mTRAV4Int | 180 | GGiTiMAGGAACAAAGG | 210 . . . 231 |
| | | AGAAT | |
| mTRAV5-15-4Int | 315 | ATYCGTTCAAATATGGA | 211 . . . 233 |
| | | AAGAAA | |
| mTRAV6-16-2Int | 174 | GGAGAAGGTCCACAGCT | 178 . . . 195 |
| | | C | |
| mTRAV6-36-4Int | 176 | CAACtGCCAACAACAAG | 209 . . . 226 |
| | | G | |
| mTRAV6-36-4Int-1 | 176 | CAACtGCCAACAACAAG | 209 . . . 226 |
| | | G | |
| mTRAV6-56-7Int | 178 | TCCTTCCACTTGCAGAA | 271 . . . 289 |
| | | AG | |
| mTRAV6-6Int | 231 | ACGGCTGGCCAGAAG | 217 . . . 231 |
| mTRAV8Int | 232 | AGAGCCACCCTTGACAC | 244 . . . 260 |
| mTRAV9Int | 233 | GCTTYGAGGCTGAGTTC | 239 . . . 257 |
| | | AG | |
| mTRAC Rev Int | 234 | GCACATTGATTTGGGAG | 100-82 |
| | | TC | |
| mTRAV1010aExt | 235 | AGAGAAGGTCGAGCAAC | 66 . . . 84 |
| | | AC | |
| mTRAV11Ext | 236 | AAGACCCAAGTGGAGCA | 64 . . . 81 |
| | | G | |
| mTRAV12Ext | 237 | TGACCCAGACAGAAGGC | 68 . . . 84 |
| mTRAV13Ext | 169 | TCCTTGGTTCTGCAGG | 88 . . . 103 |
| mTRAV14Ext | 171 | GCAGCAGGTGAGACAAA | 87 . . . 104 |
| | | G | |
| mTRAV19Ext | 238 | gcAAGttAaAcAAAGCT | 286 . . . 306 |
| | | CTCC | |
| mTRBV31Ext | 202 | ctaACcTCtacTGGTAC | 176 . . . 198 |
| | | TGGCAG | |
| mTRAV15Ext | 167 | CASCTTYTTAGTGGAGA | 175 . . . 196 |
| | | GATGG | |
| mTRAV16Ext | 181 | GTACAAGCAAACAGCAA | 168 . . . 187 |
| | | GTG | |
| mTRAV17Ext | 239 | CAGTCCGTGGACCAGC | 61 . . . 76 |
| mTRAV6-56-7Ext | 177 | gTTCTGGTAtGTGCAGT | 156 . . . 176 |
| | | ATCC | |
| mTRAV18Ext | 240 | AACGGCTGGAGCAGAG | 59 . . . 74 |
| mTRAV2Ext | 195 | caTcTACTGGTACCGAC | 147 . . . 166 |
| | | AGG | |
| mTRAV21Ext | 183 | GTGCACTTGCCTTGTAG | 103 . . . 120 |
| | | C | |
| mTRAV3Ext | 241 | GGCGAGCAGGTGGAG | 64 . . . 78 |
| mTRAV5-15-4Ext | 242 | GgcTACTTCcCtTGGTA | 154 . . . 179 |
| | | TAAGCAAGA | |
| mTRAV4Ext | 179 | TcTGSTCTGAGATGCAA | 113 . . . 133 |
| | | TTTT | |
| mTRAV6-36-4Ext | 175 | AAGGTCCACAGCTCCTT | 182 . . . 199 |
| | | C | |
| mTRAV6-36-4Ext-1 | 175 | AAGGTCCACAGCTCCTT | 182 . . . 199 |
| | | C | |
| mTRAV6-6Ext | 243 | AGATTCCGTGACTCAAA | 60 . . . 79 |
| | | CAG | |
| mTRAV7Ext | 244 | AGAAGGTRCAGCAGAGC | 65 . . . 89 |
| | | CCAGAATC | |
| mTRAV8Ext | 245 | GAGCRTCCASGAGGGTG | 93 . . . 109 |
| mTRAV9Ext | 246 | CCAGTGGTTCAAGGAGT | 217 . . . 234 |
| | | G | |
| mTRAC Rev Ext | 247 | GGCATCACAGGGAACG | 276-261 |

TABLE 10
Human αβ primer list with positions
(internal forward V region and reverse C region
specific primers and their positions).

| | SEQ | | |
|---|---|---|---|
| | ID | | |
| Primer name | NO: | Primer Sequence | Region |
| huTRAV34int | 248 | aTcTCaccATAAACTGCACG | 101 . . . 120 |
| huTRAV1int | 249 | GCACCCACATTTCTKTCTTAC | 175 . . . 195 |
| huTRAV10int | 250 | GAAAGAACTGCACTCTTCAATG | 110 . . . 131 |
| huTRAV12-1 | 251 | AAGATGGAAGGTTTACAGCAC | 230 . . . 250 |
| huTRAV13-1int | 252 | TCAGACAGTGCCTCAAACTAC | 133 . . . 153 |
| huTRAV13-2int | 253 | CAGTGAAACATCTCTCTCTGC | 266 . . . 286 |
| huTRAV14int | 254 | AGGCTGTGACTCTGGACTG | 110 . . . 128 |
| huTRAV16int | 255 | GTCCAGTACTCCAGACAACG | 166 . . . 185 |
| huTRAV17int | 256 | CCACCATGAACTGCAGTTAC | 116 . . . 135 |
| huTRAV18int | 257 | TGACAGTTCCTTCCACCTG | 261 . . . 279 |
| huTRAV19int | 258 | TGTGACCTTGGACTGTGTG | 114 . . . 132 |
| huTRAV2int | 259 | CACTCTGTGTCCAATGCTTAC | 145 . . . 165 |
| huTRAV20int | 260 | TCTGGTATAGGCAAGATCCTG | 164 . . . 184 |
| huTRAV21int | 261 | AACTTGGTTCTCAACTGCAG | 109 . . . 128 |
| huTRAV22int | 262 | CTGACTCTGTGAACAATTTGC | 137 . . . 157 |
| huTRBV3int | 263 | aATctTcaCAtCAATTCCCTG | 185 . . . 205 |
| huTRAV23int | 264 | TGCATTATTGATAGCCATACG | 216 . . . 236 |
| huTRAV24int | 265 | TGCCTTACACTGGTACAGATG | 159 . . . 179 |
| huTRAV25int | 266 | TATAAGCAAAGGCCTGGTG | 157 . . . 175 |
| huTRAV26-1int | 267 | CGACAGATTCACTCCCAG | 160 . . . 177 |
| huTRAV26-2int | 268 | TTCACTTGCCTTGTAACCAC | 104 . . . 123 |
| huTRAV27int | 269 | CTCACTGTGTACTGCAACTCC | 109 . . . 129 |
| huTRAV29int | 30 | CTGCTGAAGGTCCTACATTC | 197 . . . 216 |
| huTRAV3int | 270 | ATGCACCTATTCAGTCTCTGG | 123 . . . 143 |
| huTRAV8-24int | 271 | AGAGtgAAACCTCCTTCCAC | 263 . . . 282 |
| huTRAV30int | 272 | AGAAGCATGGTGAAGCAC | 170 . . . 187 |
| huTRAV35int | 273 | ACCTGGCTATGGTACAAGC | 145 . . . 163 |
| huTRAV36int | 34 | ATCTCTGGTTGTCCACGAG | 90 . . . 108 |
| huTRAV38int | 274 | CAGCAGGCAGATGATTCTC | 183 . . . 201 |
| huTRAV39int | 275 | TCAACCACTTCAGACAGACTG | 130 . . . 150 |
| huTRAV4int | 276 | ATTATATCACGTGGTACCAACAG | 143 . . . 165 |
| huTRAV40int | 277 | GGAGGCGGAAATATTAAAGAC | 226 . . . 246 |
| huTRAV41int | 278 | TTGTTTATGCTGAGCTCAGG | 202 . . . 221 |
| huTRAV5int | 279 | TACACAGACAGCTCCTCCAC | 133 . . . 152 |
| huTRAV6int | 280 | TGGTACCGACAAGATCCAG | 163 . . . 181 |
| huTRAV7int | 281 | TATGAGAAGCAGAAAGGAAGAC | 226 . . . 247 |
| huTRAV8-1int | 282 | GTCAACACCTTCAGCTTCTC | 179 . . . 198 |
| huTRAV8-7int | 283 | ATCAgaGGtTTTGAGGCTG | 235 . . . 253 |
| huTRAV8-6int | 284 | AACcAAGGACTCCAGCTTC | 178 . . . 196 |
| huTRAV8-3int | 285 | TTTGAGGCTGAATTTAAGAGG | 244 . . . 264 |
| huTRAV9-1 | 286 | GAAACCACTTCTTTCCACTTG | 262 . . . 282 |
| hu TRAC Rev INT | 287 | TGTTGCTCTTGAAGTCCATAG | 181-160 |

| | SEQ | | |
|---|---|---|---|
| | ID | | |
| Primer name | NO: | Primer Sequence | Region |
| huTRBV10-1int | 288 | TGGTATCGACAAGACCTGG | 157 . . . 175 |
| huTRBV10-2int | 288 | TGGTATCGACAAGACCTGG | 157 . . . 175 |
| huTRBV10-3int | 289 | GGAACACCAGTGACTCTGAG | 103 . . . 122 |
| huTRBV11int | 290 | GACTCCACTCTCAAGATCCA | 277 . . . 296 |
| huTRBV12int | 291 | CYACTCTgARGATCCAGCC | 281 . . . 299 |
| huTRBV13int | 292 | CATTCTGAACTGAACATGAGC | 304 . . . 324 |
| huTRBV5-1 | 293 | CTTGGAGCTGGRSGACTC | 327 . . . 344 |
| huTRBV14int | 294 | ATTCTACTCTGAAGGTGCAGC | 278 . . . 298 |
| huTRBV15int | 295 | ATAACTTCCAATCCAGGAGG | 242 . . . 261 |
| huTRAV4int | 276 | aTTATaTcacgTGGTACCAACAG | 146 . . . 168 |
| huTRBV16int1 | 296 | CTGTAGCCTTGAGATCCAGG | 279 . . . 298 |
| huTRBV17int | 297 | TGTTCACTGGTACCGACAG | 150 . . . 168 |
| huTRBV18int | 298 | CGATTTTCTGCTGAATTTCC | 247 . . . 266 |
| huTRBV19int | 299 | TTCCTCTCACTGTGACATCG | 278 . . . 297 |
| huTRBV2int | 300 | TTCACTCTGAAGATCCGGTC | 280 . . . 299 |
| huTRBV20int | 301 | ACTCTGACAGTGACCAGTGC | 307 . . . 326 |
| huTRBV23int | 302 | GCAATCCTGTCCTCAGAAC | 289 . . . 307 |
| huTRBV24int | 303 | GATGGATACAGTGTCTCTCGA | 241 . . . 261 |
| huTRAV6int | 280 | TGGTAcCgACAAGATCCAG | 157 . . . 175 |
| huTRBV25int | 304 | CAGAGAAGGGAGATCTTTCC | 221 . . . 240 |
| huTRBV2728int | 305 | TTCYCCCTGATYCTGGAGTC | 277 . . . 296 |
| huTRBV29int | 306 | TCTGACTGTGAGCAACATGAG | 276 . . . 296 |
| huTRBV3int | 263 | AATCTTCACATCAATTCCCTG | 280 . . . 300 |
| huTRBV30int | 307 | AGAATCTCTCAGCCTCCAGAC | 236 . . . 256 |
| huTRBV4int | 308 | CCTGCAGCCAGAAGACTC | 297 . . . 314 |
| huTRBV5-5 | 309 | TCTGAGCTGAATGTGAACG | 277 . . . 295 |
| huTRBV6-1 | 310 | GTGTRCCCAGGATATGAACC | 123 . . . 142 |
| huTRBV6-4int | 311 | TGGTTATAGTGTCTCCAGAGC | 243 . . . 263 |
| huTRBV7-1 | 312 | TCYACTCTGAMGWTCCAGCG | 280 . . . 299 |
| huTRBV9int | 313 | GTACCAACAGAGCCTGGAC | 159 . . . 177 |
| huTRBC Rev Int | 314 | TTCTGATGGCTCAAACACAG | 54-35 |

TABLE 11
Mouse γδ primer list with positions.

| | SEQ | | SEQ | | |
|---|---|---|---|---|---|
| | ID | | ID | | |
| Primer name | NO: | External | NO: | Internal | Region |
| TRGV1-3 | 147 | GCAGCTGGAGCAAACTG | 148 | CTGAATTATCGGTCACCAG | 68-86 |
| TRGV4 | 149 | CAAATATCCTGTAAAGTTTTCA | 150 | GTTTAGAGTTTCTATTATATGTCCTT | 251-281 |
| | | TC | | GCAAC | |
| TRGV5 | 151 | GATATCTCAGGATCAGCTCTCC | 152 | TACCCGAAGACCAAACAAGAC | 81-101 |
| TRGV6 | 153 | TCACCTCTGGGGTCATATG | 154 | AGAGGAAAGGAAATACGGC | 137-155 |
| TRGV7 | 155 | CAACTTGGAAGAAAGAATAAT | 156 | CACCAAGCTAGAGGGGTC | 87-104 |
| | | GTC | | | |
| TRGC | 157 | CTTTTCTTTCCAATACACCC | 158 | TCDGGAAAGAACTTTTCAAGG | 118-98 |
| (reverse) | | | | | |
| TRDV1 | 159 | ACCCAAATGTTGCATCAG | 160 | GTCTCTGACAATCCAAGAAGG | 87-107 |
| TRDV2 | 161 | TCTGTGCAGGTGGCAG | 162 | CCCTGGACTGCACCTATG | 119-136 |
| TRDV4 | 163 | TGTATATTTGGAACCAGTTGC | 164 | GATCCTGCCTCCTTCTACTG | 106-125 |
| TRDV5 | 165 | CATCACGCTGACCCAG | 166 | GCTCCACTGACCAGACAG | 71-88 |
| TRDV6/ | 167 | CASCTTYTTAGTGGAGAGATG | 168 | AYTCTGTAGTCTTCCAGAAATCAC | 251-274 |
| TRAV15 | | G | | | |
| TRDV7/ | 169 | TCCTTGGTTCTGCAGG | 170 | TGCAGGAGGGGGAGA | 98-112 |
| TRAV13 | | | | | |
| TRDV8/ | 171 | GCAGCAGGTGAGACAAAG | 172 | CTCTGACAGTCTGGGAAGG | 113-131 |
| TRAV14 | | | | | |
| TRDV9/ | 173 | CAGATGCAAGGTCAAGTGAC | 174 | GGAGAAGGTCCACAGCTC | 178-195 |
| TRAV6-1/6-2 | | | | | |
| TRDV9/ | 175 | AAGGTCCACAGCTCCTTC | 176 | CAACTGCCAACAACAAGG | 209-226 |
| TRAV6-3/6-4 | | | | | |
| TRDV9/ | 177 | GTTCTGGTATGTGCAGTATCC | 178 | TCCTTCCACTTGCAGAAAG | 271-289 |
| TRAV6-5/6-7 | | | | | |
| TRDV10/ | 179 | TCTGSTCTGAGATGCAATTTT | 180 | GGITIMAGGAACAAAGGAGAAT | 210-231 |
| TRAV4 | | | | | |
| TRDV11/ | 181 | GTACAAGCAAACAGCAAGTG | 182 | ATTATTCTCTGAACTTTCAGAAGC | 248-271 |
| TRAV16 | | | | | |
| TRDV12/ | 183 | GTGCACTTGCCTTGTAGC | 184 | AATAGTATGGCTTTCCTGGC | 220-239 |
| TRAV21 | | | | | |
| TRDC | 185 | TGAAAGAATTTTGCATATGGTT | 186 | GAGATGACTATAGCAGGGTCG | 151-131 |
| (reverse) | | C | | | |

### REFERENCES

1. Restifo, N P, Dudley, M E and Rosenberg, S A (2012). Adoptive immunotherapy for cancer: harnessing the T cell response. Nat. Rev. Immunol. 12:269-281.
2. Rosenberg, S A and Restifo, N P (2015). Adoptive cell transfer as personalized immunotherapy for human cancer. Science 348:62-68.
3. Wherry, E J, Ha, S-J, Kaech, S M, Haining, W N, Sarkar, S, Kalia, V, et al. (2007). Molecular Signature of CD8+ T Cell Exhaustion during Chronic Viral Infection. Immunity 27:670-684.
4. Baitsch, L, Baumgaertner, P, Devêvre, E, Raghav, S K, Legat, A, Barba, L, et al. (2011). Exhaustion of tumor-specific CD8+ T cells in metastases from melanoma patients. J. Clin. Invest. 121:2350-2360.
5. Wherry, E J and Kurachi, M (2015). Molecular and cellular insights into T cell exhaustion. Nat. Rev. Immunol. 15:486-499.
6. Besser, M J, Shapira-Frommer, R, Itzhaki, O, Treves, A J, Zippel, D B, Levy, D, et al. (2013). Adoptive Transfer of Tumor-Infiltrating Lymphocytes in Patients with Metastatic Melanoma: Intent-to-Treat Analysis and Efficacy after Failure to Prior Immunotherapies. Clin. Cancer Res. 19:4792-4800.
7 Klaver, Y, Kunert, A, Sleijfer, S, Debets, R and Lamers, C H (2015). Adoptive T-cell therapy: a need for standard immune monitoring. Immunotherapy 7:513-533.
8. Lamers, C H J, Willemsen, R, van Elzakker, P, van Steenbergen-Langeveld, S, Broertjes, M, Oosterwijk-Wakka, J, et al. (2011). Immune responses to transgene and retroviral vector in patients treated with ex vivo-engineered T cells. Blood 117:72-82.
9. Chapuis, A G, Thompson, J A, Margolin, K A, Rodmyre, R, Lai, I P, Dowdy, K, et al. (2012). Transferred melanoma-specific CD8+ T cells persist, mediate tumor regression, and acquire central memory phenotype. Proc. Natl. Acad. Sci. U.S.A 109:4592-4597.
10. Han, E Q, Li, X, Wang, C, Li, T and Han, S (2013). Chimeric antigen receptor-engineered T cells for cancer immunotherapy: progress and challenges. J. Hematol. Oncol. J Hematol Oncol 6:47.
11. Hinrichs, C S, Borman, Z A, Cassard, L, Gattinoni, L, Spolski, R, Yu, Z, et al. (2009). Adoptively transferred effector cells derived from naïve rather than central memory CD8+ T cells mediate superior antitumor immunity. Proc. Natl. Acad. Sci. U.S.A 106:17469-17474.
12. Kershaw, M H, Westwood, J A, Parker, L L, Wang, G, Eshhar, Z, Mavroukakis, S A, et al. (2006). A Phase I Study on Adoptive Immunotherapy Using Gene-Modified T Cells for Ovarian Cancer. Clin. Cancer Res. 12:6106-6115.
13. Ramos, C A and Dotti, G (2011). Chimeric antigen receptor (CAR)-engineered lymphocytes for cancer therapy. Expert Opin. Biol. Ther. 11:855-873.
14. Varela-Rohena, A, Carpenito, C, Perez, E E, Richardson, M, Parry, R V, Milone, M, et al. (2008). Genetic engineering of T cells for adoptive immunotherapy. Immunol. Res. 42:166-181.
15. Schmitt, T M, Ragnarsson, G B and Greenberg, P D (2009). T Cell Receptor Gene Therapy for Cancer. Hum. Gene Ther. 20:1240-1248.
16. Stauss, H J, Morris, E C and Abken, H (2015). Cancer gene therapy with T cell receptors and chimeric antigen receptors. Curr. Opin. Pharmacol. 24:113-118.
17. Schmitt, T M, Stromnes, I M, Chapuis, A G and Greenberg, P D (2015). New Strategies in Engineering T-cell Receptor Gene-Modified T Cells to More Effectively Target Malignancies. Clin. Cancer Res.: clincanres.0860.2015doi: 10.1158/1078-0432.CCR-15-0860.
18. Vavassori, S, Kumar, A, Wan, G S, Ramanjaneyulu, G S, Cavallari, M, Daker, S El, et al. (2013). Butyrophilin 3A1 binds phosphorylated antigens and stimulates human γδ T cells. Nat. Immunol. 14:908-916.
19. Birnbaum, M E, Mendoza, J L, Sethi, D K, Dong, S, Glanville, J, Dobbins, J, et al. (2014). Deconstructing the peptide-MHC specificity of T cell recognition. Cell 157:1073-1087.
20. Luoma, A M, Castro, C D, Mayassi, T, Bembinster, L A, Bai, L, Picard, D, et al. (2013). Crystal structure of V81 T cell receptor in complex with CD1d-sulfatide shows MHC-like recognition of a self-lipid by human γδ T cells. Immunity 39:1032-1042.
21. Linnemann, C, Heemskerk, B, Kvistborg, P, Kluin, R J C, Bolotin, D A, Chen, X, et al. (2013). High-throughput identification of antigen-specific TCRs by TCR gene capture. Nat. Med. 19:1534-1541.
22. Kobayashi, E, Mizukoshi, E, Kishi, H, Ozawa, T, Hamana, H, Nagai, T, et al. (2013). A new cloning and expression system yields and validates TCRs from blood lymphocytes of patients with cancer within 10 days. Nat. Med. 19:1542-1546.
23. Howie, B, Sherwood, A M, Berkebile, A D, Berka, J, Emerson, R O, Williamson, D W, et al. (2015). High-throughput pairing of T cell receptor a and B sequences. Sci. Transl. Med. 7: 301ra131.
24. Perko, R, Kang, G, Sunkara, A, Leung, W, Thomas, P G and Dallas, M H (2015). Gamma Delta T Cell Reconstitution Is Associated with Fewer Infections and Improved Event-Free Survival after Hematopoietic Stem Cell Transplantation for Pediatric Leukemia. Biol. Blood Marrow Transplant. 21:130-136.
25. Silva-Santos, B, Serre, K and Norell, H (2015). γδ T cells in cancer. Nat. Rev. Immunol. advance online publication.
26. Dash, P, McClaren, J L, Oguin, T H, Rothwell, W, Todd, B, Morris, M Y, et al. (2011). Paired analysis of TCRα and TCRβ chains at the single-cell level in mice. J. Clin. Invest. 121:288-295.
27. Wang, G C, Dash, P, McCullers, J A, Doherty, P C and Thomas, P G (2012). T cell receptor αβ diversity inversely correlates with pathogen-specific antibody levels in human cytomegalovirus infection. Sci. Transl. Med. 4: 128ra42.
28. Bryksin, A V and Matsumura, I (2010). Overlap extension PCR cloning: a simple and reliable way to create recombinant plasmids. BioTechniques 48:463-465.
29. Uldrich, A P, Le Nours, J, Pellicci, D G, Gherardin, N A, McPherson, K G, Lim, R T, et al. (2013). CD1d-lipid antigen recognition by the γδ TCR. Nat. Immunol. 14:1137-1145.
30. Yoshio Ogawa, M N (2013). Gamma-delta T Cells may Function as Carrier Vehicles in Adenovirus Vector-based Gene Therapy. J. Cancer Sci. Ther. 05.
31. (2006). Generation of T-cell receptor retrogenic mice: Article: Nature Protocols. Nat Protoc. 1:406-417.
32. Bettini, M L, Bettini, M, Nakayama, M, Guy, C S and Vignali, D A A (2013). Generation of T cell receptor-retrogenic mice: improved retroviral-mediated stem cell gene transfer. Nat. Protoc. 8:1837-1840.
33. Sharma, P, Yan, F, Doronina, V A, Escuin-Ordinas, H, Ryan, M D and Brown, J D (2012). 2A peptides provide distinct solutions to driving stop-carry on translational recoding. Nucleic Acids Res. 40:3143-3151.
34. Holst, J, Szymczak-Workman, A L, Vignali, K M, Burton, A R, Workman, C J and Vignali, D A A (2006). Generation of T-cell receptor retrogenic mice. Nat. Protoc. 1:406-417.
35. Szymczak, A L, Workman, C J, Wang, Y, Vignali, K M, Dilioglou, S, Vanin, E F, et al. (2004). Correction of multi-gene deficiency in vivo using a single ‘self-cleaving’ 2A peptide-based retroviral vector. Nat. Biotechnol. 22:589-594.
36. Bettini, M L, Bettini, M and Vignali, D A A (2012). T-cell receptor retrogenic mice: a rapid, flexible alternative to T-cell receptor transgenic mice. Immunology 136:265-272.
37. Day, E B, Guillonneau, C, Gras, S, La Gruta, N L, Vignali, DAA, Doherty, P C, et al. (2011). Structural basis for enabling T-cell receptor diversity within biased virus-specific CD8+ T-cell responses. Proc. Natl. Acad. Sci. U.S.A 108:9536-9541.
38. Bartok, I, Holland, S J, Kessels, H W, Silk, J D, Alkhinji, M and Dyson, J (2010). T cell receptor CDR3 loops influence alphabeta pairing. Mol. Immunol. 47:1613-1618.
39. Siegers, G M, Swamy, M, Fernández-Malavé, E, Minguet, S, Rathmann, S, Guardo, A C, et al. (2007). Different composition of the human and the mouse γδ T cell receptor explains different phenotypes of CD3γ and CD3δ immunodeficiencies. J. Exp. Med. 204:2537-2544.
40. Moran, A E, Holzapfel, K L, Xing, Y, Cunningham, N R, Maltzman, J S, Punt, J, et al. (2011). T cell receptor signal strength in Treg and iNKT cell development demonstrated by a novel fluorescent reporter mouse. J. Exp. Med. 208:1279-1289.
41. Gao, P, Han, X, Zhang, Q, Yang, Z, Fuss, I J, Myers, T G, et al. (2014). Dynamic changes in E-protein activity regulate T reg cell development. J. Exp. Med. 211:2651-2668.
42. Au-Yeung, B B, Zikherman, J, Mueller, J L, Ashouri, J F, Matloubian, M, Cheng, D A, et al. (2014). A sharp T-cell antigen receptor signaling threshold for T-cell proliferation. Proc. Natl. Acad. Sci. 111: E3679-E3688.
43. Peng, H, Sohara, Y, Moats, R A, Nelson, M D, Groshen, S G, Ye, W, et al. (2007). The Activity of Zoledronic Acid on Neuroblastoma Bone Metastasis Involves Inhibition of Osteoclasts and Tumor Cell Survival and Proliferation. Cancer Res. 67:9346-9355.
44. Bäckman, U, Svensson, Å, Christofferson, R H and Azarbayjani, F (2008). The Bisphosphonate, Zoledronic Acid Reduces Experimental Neuroblastoma Growth by Interfering with Tumor Angiogenesis. Anticancer Res. 28:1551-1557.
45. Gnant, M and Clezardin, P (2012). Direct and indirect anticancer activity of bisphosphonates: A brief review of published literature. Cancer Treat. Rev. 38:407-415.
46. Di Carlo, E, Bocca, P, Emionite, L, Cilli, M, Cipollone, G, Morandi, F, et al. (2013). Mechanisms of the Antitumor Activity of Human Vγ9Vδ2 T Cells in Combination With Zoledronic Acid in a Preclinical Model of Neuroblastoma. Mol. Ther. 21:1034-1043.
47. Goto, H, Matsuda, K, Srikoon, P, Kariya, R, Hattori, S, Taura, M, et al. (2013). Potent antitumor activity of zoledronic acid-induced Vγ9Vδ2 T cells against primary effusion lymphoma. Cancer Lett. 331:174-182.
48. Sprini, D, Di Stefano, L, Rini, G B, Cianferotti, L and Napoli, N (2014). Vγ9Vδ2 T lymphocytes activation in osteoporotic patients treated with bisphosphonates. Clin. Cases Miner. Bone Metab. 11:126-128.
49. Wada, I, Matsushita, H, Noji, S, Mori, K, Yamashita, H, Nomura, S, et al. (2014). Intraperitoneal injection of in vitro expanded Vγ9Vδ2 T cells together with zoledronate for the treatment of malignant ascites due to gastric cancer. Cancer Med. 3:362-375.
50. Suzuki, T, Terao, S, Acharya, B, Naoe, M, Yamamoto, S, Okamura, H, et al. (2010). The Antitumour Effect of γδ T-Cells is Enhanced by Valproic Acid-induced Up-regulation of NKG2D Ligands. Anticancer Res. 30:4509-4513.
51. Pardoll, D M (2012). The blockade of immune checkpoints in cancer immunotherapy. Nat. Rev. Cancer 12:252-264.
52. Dudley, M E, Wunderlich, J R, Shelton, T E, Even, J and Rosenberg, S A (2003). Generation of Tumor-Infiltrating Lymphocyte Cultures for Use in Adoptive Transfer Therapy for Melanoma Patients. J. Immunother. Hagerstown Md 1997 26:332-342.
53. Chien, Y, Meyer, C and Bonneville, M (2014). γδ T Cells: First Line of Defense and Beyond. Annu. Rev. Immunol. 32:121-155.
54. Gros, A, Robbins, P F, Yao, X, Li, Y F, Turcotte, S, Tran, E, et al. (2014). PD-1 identifies the patient-specific CD8+ tumor-reactive repertoire infiltrating human tumors. J. Clin. Invest. 124:2246-2259.
55. Downing, J R, Wilson, R K, Zhang, J, Mardis, E R, Pui, C-H, Ding, L, et al. (2012). The Pediatric Cancer Genome Project. Nat. Genet. 44:619-622.
56. Robinson, G, Parker, M, Kranenburg, T A, Lu, C, Chen, X, Ding, L, et al. (2012). Novel mutations target distinct subgroups of medulloblastoma. Nature 488:43-48.
57. Zhang, J, Benavente, C A, McEvoy, J, Flores-Otero, J, Ding, L, Chen, X, et al. (2012). A novel retinoblastoma therapy from genomic and epigenetic analyses. Nature 481:329-334.
58. Zhang, J, Ding, L, Holmfeldt, L, Wu, G, Heatley, S L, Payne-Turner, D, et al. (2012). The genetic basis of early T-cell precursor acute lymphoblastic leukaemia. Nature 481:157-163.
59. Barrett, A J and Bollard, C M (2015). The coming of age of adoptive T-cell therapy for viral infection after stem cell transplantation. Ann. Transl. Med. 3.
60. Saglio, F, Hanley, P J and Bollard, C M (2014). The time is now: moving toward virus-specific T cells after allogeneic hematopoietic stem cell transplantation as the standard of care. Cytotherapy 16:149-159.
61. Vantourout, P and Hayday, A (2013). Six-of-the-best: unique contributions of γδ T cells to immunology. Nat. Rev. Immunol. 13:88-100.
62. Lefranc, M-P, Giudicelli, V, Ginestoux, C, Jabado-Michaloud, J, Folch, G, Bellahcene, F, et al. (2009). IMGT, the international ImMunoGeneTics information system. Nucleic Acids Res. 37: D1006-1012.
63. Bell, J (2008). A simple way to treat PCR products prior to sequencing using ExoSAP-IT. BioTechniques 44:834.
64. Workman, C J, Dugger, K J and Vignali, D A A (2002). Cutting Edge: Molecular Analysis of the Negative Regulatory Function of Lymphocyte Activation Gene-3. J. Immunol. 169:5392-5395.
65. Persons, D A, Allay, J A, Allay, E R, Smeyne, R J, Ashmun, R A, Sorrentino, B P, et al. (1997). Retroviral-Mediated Transfer of the Green Fluorescent Protein Gene Into Murine Hematopoietic Cells Facilitates Scoring and Selection of Transduced Progenitors In Vitro and Identification of Genetically Modified Cells In Vivo. Blood 90:1777-1786.
66. Heemskerk, MHM, Hoogeboom, M, Paus, R A de, Kester, MGD, van der Hoorn, MAWG, Goulmy, E, et al. (2003). Redirection of antileukemic reactivity of peripheral T lymphocytes using gene transfer of minor histocompatibility antigen HA-2-specific T-cell receptor complexes expressing a conserved alpha joining region. Blood 102:3530-3540.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

LIST OF SEQUENCES
HuTRGV3.5
External Primer:
(SEQ ID NO: 1)
5′TCTTCCAACTTGGAAGGG3′
Internal Primer:
(SEQ ID NO: 2)
5′GGTCATCTGCTGAAATCAC3′
HuTRGV7
External Primer:
(SEQ ID NO: 3)
5′TCTTCCAACTTGCAAGGG3′
Internal Primer:
(SEQ ID NO: 4)
5′GGTCATCTGCTGTAATCACTTG3′
HuTRGVA
External Primer:
(SEQ ID NO: 5)
5′GGGTCATCCTGTTTCCAG3′
Internal Primer:
(SEQ ID NO: 6)
5′TACCTAAGGACCTGTGTAGAGG3′
HuTRGVB
External Primer:
(SEQ ID NO: 7)
5′TGGCCTCCCAAAGTACTG3′
Internal Primer:
(SEQ ID NO: 8)
5′TCCTCTTTCTATGTCCCAGG3′
HuTRGV8
External Primer:
(SEQ ID NO: 9)
5′CCAACTTGGAAGGGAGAAC3′
Internal Primer:
(SEQ ID NO: 10)
5′AAAATGCCGTCTACACCC3′
HuTRGV9
External Primer:
(SEQ ID NO: 11)
5′CCAGGTCACCTAGAGCAAC3′
Internal Primer:
(SEQ ID NO: 12)
5′TGTCCATTTCATATGACGG3′
HuTRGV10
External Primer:
(SEQ ID NO: 13)
5′TTATCAAAAGTGGAGCAGTTC3′
Internal Primer:
(SEQ ID NO: 14)
5′CAGCTATCCATTTCCACGG3′
HuTRGV11
External Primer:
(SEQ ID NO: 15)
5′GAACAACCTGAAATATCTATTTCC3′
Internal Primer:
(SEQ ID NO: 16)
5′CATATCTTGGAAGGCATCC3′
HuTRGV1.2.4.6
External Primer:
(SEQ ID NO: 17)
5′GGGTCATCTGCTGAAATCAC3′
Internal Primer:
(SEQ ID NO: 18)
5′CCAGGAGGGGAAGGC3′
HuTRGC
External Primer:
5′GGTGTTCCCCTCCTGG3′
Internal Primer:
(SEQ ID NO: 20)
5′CCCAGAATCGTGTTGCT3′
HuTRDV1
External Primer:
(SEQ ID NO: 21)
5′GCCCAGAAGGTTACTCAAG3′
Internal Primer:
(SEQ ID NO: 22)
5′AGCAAAGAGATGATTTTCCTTA3′
HuTRDV2
External Primer:
(SEQ ID NO: 23)
5′ATTGAGTTGGTGCCTGAAC3′
Internal Primer:
(SEQ ID NO: 24)
5′TATATCAACTGGTACAGGAAGACC3′
HuTRDV3
External Primer:
(SEQ ID NO: 25)
5′TGTGACAAAGTAACCCAGAGTTC3′
Internal Primer:
(SEQ ID NO: 26)
5′GGTACTGCTCTGCACTTACGAC3′
HuTRDV4/TRAV14
External Primer:
(SEQ ID NO: 27)
5′CAAACCCAACCAGGAATG3′
Internal Primer:
(SEQ ID NO: 28)
5′AGGAAAAGGAGGCTGTGAC3′
HuTRDV5/TRAV29
External Primer:
(SEQ ID NO: 29)
5′GCAAGTTAAGCAAAATTCACC3′
Internal Primer:
(SEQ ID NO: 30)
5′CTGCTGAAGGTCCTACATTC3′
HuTRDV6/TRAV23
External Primer:
(SEQ ID NO: 31)
5′TTGATAGTCCAGAAAGGAGG3′
Internal Primer:
(SEQ ID NO: 32)
5′CGTTTGACTACTTTCCATGG3′
HuTRDV7/TRAV36
External Primer:
(SEQ ID NO: 33)
5′GACAAGGTGGTACAAAGCC3′
Internal Primer:
(SEQ ID NO: 34)
5′ATCTCTGGTTGTCCACGAG3′
HuTRDV8/TRAV38-2
External Primer:
(SEQ ID NO: 35)
5′CAGTCACTCAGTCTCAACCAG3′
Internal Primer:
(SEQ ID NO: 36)
5′TCTGGTACAAGCAGCCTC3′
HuTRDC
External Primer:
(SEQ ID NO: 37)
5′CTTCATATTTACCAAGCTTGACAG3′
Internal Primer:
(SEQ ID NO: 38)
5′GATGACAATAGCAGGATCAAAC3′
CD38δ sense
(SEQ ID NO: 39)
5′CCCTCACTCCTTCTCTAGGCGCCGGAATTCGCCAGGATGGAACATAGCACG3′
CD3δ antisense
(SEQ ID NO: 40)
5′CCACGTCTCCCGCCAACTTGAGAAGGTCAAAATTCAAAGTCTGTTTCACCGGTCCC
TTGTTCCGAGCC3′
CD3γ sense
(SEQ ID NO: 41)
5′GAATTTTGACCTTCTCAAGTTGGCGGGAGACGTGGAGTCCAACCCAGGGCCCATGG
AACAGGGGAAG3′
CD3γ antisense
(SEQ ID NO: 42)
5′CCTCGACGTCACCGCATGTTAGCAGACTTCCTCTGCCCTCAGATCTTCTATTCCTCCT
CAAC3′
CD3ε sense
(SEQ ID NO: 43)
5′CAGAGGAAGTCTGCTAACATGCGGTGACGTCGAGGAGAATCCTGGCCCAATGCAG
TCGGGCACTC3′
CD3ε antisense
(SEQ ID NO: 44)
5′GTTTTCTTCCACGTCTCCTGCTTGCTTTAACAGAGAGAAGTTCGTGGCGGATCCTCC
GATGCGTCTCTG3′
CD3ζ sense
(SEQ ID NO: 45)
5′CTCTCTGTTAAAGCAAGCAGGAGACGTGGAAGAAAACCCCGGTCCCATGAAGTGG
AAAGTG3′
CD3ζ antisense
(SEQ ID NO: 46)
5′GAGGGAGAGGGGCGGAATTGATCCTCGAGCAATTGTTAGCGAGGGGCCAG3′
2A amino acid sequence F2A (foot-and-mouth disease virus)
(SEQ ID NO: 47)
VKQTLNFDLLKLAGDVESNPGP
2A amino acid sequence T2A (Thosea asigna virus)
(SEQ ID NO: 48)
EGRGSLLTCGDVEENPGP
2A amino acid sequence P2A (porcine teschovirus-1)
(SEQ ID NO: 49)
ATNFSLLKQAGDVEENPGP
HuLinkerDV1
(SEQ ID NO: 50)
5′catacctttgtcttcttgagaaatttttcccagatattattaagatacattggcaagaaaagaagagcaacacgattctgggatcccaggagg
ggaacaccatgaagactaacgacacatacatgaaatttagctggttaacggtgccagaagagtcactggacaaagaacacagatgtatcgt
cagacatgagaataataaaaacggaattgatcaagaaattatctttcctccaataaagacagatgtcatcacaatggatcccaaagacaattg
gtcaaaagatgcaaatgatacactactgctgcagctcacaaacacctctgcatattacatgtacctcctcctgctcctcaagagtgtggtctattt
tgccatcatcacctgctgtctgcttggaagaacggctttctgctgcaatggagagaaatcaGCCACGAACTTCTCTCTGTT
AAAGCAAGCAGGAGACGTGGAAGAAAACCCCGGTCCCATGCTGTTCTCCAGCCTGC
TGTGTGTATTTGTGGCCTTCAGCTACTCTGGATCAAGTGTGGCCCAGAAGGTTACTCA
AGCCCAGTCATCAGTATCCATGCCAGTGAGGAAAGCAGTCACCCTGAACTGCCTGTA
TGAAACAAGTTGGTGGTCATATTATATTTTTTGGTACAAGCAACTTCCCAGCAAAGAG
ATGATTTTCCTTATTCGCC3′
HuLinkerDV2
(SEQ ID NO: 51)
5′catacctttgtcttcttgagaaatttttcccagatattattaagatacattggcaagaaaagaagagcaacacgattctgggatcccaggagg
ggaacaccatgaagactaacgacacatacatgaaatttagctggttaacggtgccagaagagtcactggacaaagaacacagatgtatcgt
cagacatgagaataataaaaacggaattgatcaagaaattatctttcctccaataaagacagatgtcatcacaatggatcccaaagacaattg
gtcaaaagatgcaaatgatacactactgctgcagctcacaaacacctctgcatattacatgtacctcctcctgctcctcaagagtgtggtctattt
tgccatcatcacctgctgtctgcttggaagaacggctttctgctgcaatggagagaaatcaGCCACGAACTTCTCTCTGTT
AAAGCAAGCAGGAGACGTGGAAGAAAACCCCGGTCCCATGCAGAGGATCTCCTCCC
TCATCCATCTCTCTCTCTTCTGGGCAGGAGTCATGTCAGCCATTGAGTTGGTGCCTGA
ACACCAAACAGTGCCTGTGTCAATAGGGGTCCCTGCCACCCTCAGGTGCTCCATGAA
AGGAGAAGCGATCGGTAACTACTATATCAACTGGTACAGGAAGACCCAAGG3′
HuLinkerDV3
(SEQ ID NO: 52)
5′catacctttgtcttcttgagaaatttttcccagatattattaagatacattggcaagaaaagaagagcaacacgattctgggatcccaggagg
ggaacaccatgaagactaacgacacatacatgaaatttagctggttaacggtgccagaagagtcactggacaaagaacacagatgtatcgt
cagacatgagaataataaaaacggaattgatcaagaaattatctttcctccaataaagacagatgtcatcacaatggatcccaaagacaattg
gtcaaaagatgcaaatgatacactactgctgcagctcacaaacacctctgcatattacatgtacctcctcctgctcctcaagagtgtggtctattt
tgccatcatcacctgctgtctgcttggaagaacggctttctgctgcaatggagagaaatcaGCCACGAACTTCTCTCTGTT
AAAGCAAGCAGGAGACGTGGAAGAAAACCCCGGTCCCatgattcttactgtgggctttagctttttgtttttc
tacaggggcacgctgtgtgacaaagtaacccagagttccccggaccagacggtggcgagtggcagtgaggtggtactgctctgcacttac
gacactg3′
HuLinkerDV4
(SEQ ID NO: 53)
5′catacctttgtcttcttgagaaatttttcccagatattattaagatacattggcaagaaaagaagagcaacacgattctgggatcccaggagg
ggaacaccatgaagactaacgacacatacatgaaatttagctggttaacggtgccagaagagtcactggacaaagaacacagatgtatcgt
cagacatgagaataataaaaacggaattgatcaagaaattatctttcctccaataaagacagatgtcatcacaatggatcccaaagacaattg
gtcaaaagatgcaaatgatacactactgctgcagctcacaaacacctctgcatattacatgtacctcctcctgctcctcaagagtgtggtctattt
tgccatcatcacctgctgtctgcttggaagaacggctttctgctgcaatggagagaaatcaGCCACGAACTTCTCTCTGTT
AAAGCAAGCAGGAGACGTGGAAGAAAACCCCGGTCCCATGTCACTTTCTAGCCTGCT
GAAGGTGGTCACAGCTTCACTGTGGCTAGGACCTGGCATTGCCCAGAAGATAACTCA
AACCCAACCAGGAATGTTCGTGCAGGAAAAGGAGGCTGTGACTCTGG3′
HuLinkerDV5
(SEQ ID NO: 54)
5′catacctttgtcttcttgagaaatttttcccagatattattaagatacattggcaagaaaagaagagcaacacgattctgggatcccaggagg
ggaacaccatgaagactaacgacacatacatgaaatttagctggttaacggtgccagaagagtcactggacaaagaacacagatgtatcgt
cagacatgagaataataaaaacggaattgatcaagaaattatctttcctccaataaagacagatgtcatcacaatggatcccaaagacaattg
gtcaaaagatgcaaatgatacactactgctgcagctcacaaacacctctgcatattacatgtacctcctcctgctcctcaagagtgtggtctattt
tgccatcatcacctgctgtctgcttggaagaacggctttctgctgcaatggagagaaatcaGCCACGAACTTCTCTCTGTT
AAAGCAAGCAGGAGACGTGGAAGAAAACCCCGGTCCCATGGCCATGCTCCTGGGGG
CATCAGTGCTGATTCTGTGGCTTCAGCCAGACTGGGTAAACAGTCAACAGAAGAATG
ATGACCAGCAAGTTAAGCAAAATTCACCATCCCTGAGCGTCCAGGAAGGAAGAATTT
CTATTCTGAACTGTGACTATACTAACAGCATGTTTGATTATTTCCTATGGTACAAAAAAT
ACCCTGCTGAAGGTCCTACATTCCTGATATC3′
HuLinkerDV6
(SEQ ID NO: 55)
5′catacctttgtcttcttgagaaatttttcccagatattattaagatacattggcaagaaaagaagagcaacacgattctgggatcccaggagg
ggaacaccatgaagactaacgacacatacatgaaatttagctggttaacggtgccagaagagtcactggacaaagaacacagatgtatcgt
cagacatgagaataataaaaacggaattgatcaagaaattatctttcctccaataaagacagatgtcatcacaatggatcccaaagacaattg
gtcaaaagatgcaaatgatacactactgctgcagctcacaaacacctctgcatattacatgtacctcctcctgctcctcaagagtgtggtctattt
tgccatcatcacctgctgtctgcttggaagaacggctttctgctgcaatggagagaaatcaGCCACGAACTTCTCTCTGTT
AAAGCAAGCAGGAGACGTGGAAGAAAACCCCGGTCCCATGGACAAGATCTTAGGAG
CATCATTTTTAGTTCTGTGGCTTCAACTATGCTGGGTGAGTGGCCAACAGAAGGAGAA
AAGTGACCAGCAGCAGGTGAAACAAAGTCCTCAATCTTTGATAGTCCAGAAAGGAG
GGATTTCAATTATAAACTGTGCTTATGAGAACACTGCGTTTGACTACTTTCCATGGTACC3′
HuLinkerDV7
(SEQ ID NO: 56)
5′catacctttgtcttcttgagaaatttttcccagatattattaagatacattggcaagaaaagaagagcaacacgattctgggatcccaggagg
ggaacaccatgaagactaacgacacatacatgaaatttagctggttaacggtgccagaagagtcactggacaaagaacacagatgtatcgt
cagacatgagaataataaaaacggaattgatcaagaaattatctttcctccaataaagacagatgtcatcacaatggatcccaaagacaattg
gtcaaaagatgcaaatgatacactactgctgcagctcacaaacacctctgcatattacatgtacctcctcctgctcctcaagagtgtggtctattt
tgccatcatcacctgctgtctgcttggaagaacggctttctgctgcaatggagagaaatcaGCCACGAACTTCTCTCTGTT
AAAGCAAGCAGGAGACGTGGAAGAAAACCCCGGTCCCATGATGAAGTGTCCACAGG
CTTTACTAGCTATCTTTTGGCTTCTACTGAGCTGGGTGAGCAGTGAAGACAAGGTGGT
ACAAAGCCCTCTATCTCTGGTTGTCCACGAGGGAG3′
HuLinkerDV8
(SEQ ID NO: 57)
5′catacctttgtcttcttgagaaatttttcccagatattattaagatacattggcaagaaaagaagagcaacacgattctgggatcccaggagg
ggaacaccatgaagactaacgacacatacatgaaatttagctggttaacggtgccagaagagtcactggacaaagaacacagatgtatcgt
cagacatgagaataataaaaacggaattgatcaagaaattatctttcctccaataaagacagatgtcatcacaatggatcccaaagacaattg
gtcaaaagatgcaaatgatacactactgctgcagctcacaaacacctctgcatattacatgtacctcctcctgctcctcaagagtgtggtctattt
tgccatcatcacctgctgtctgcttggaagaacggctttctgctgcaatggagagaaatcaGCCACGAACTTCTCTCTGTT
AAAGCAAGCAGGAGACGTGGAAGAAAACCCCGGTCCCATGGCATGCCCTGGCTTCC
TGTGGGCACTTGTGATCTCCACCTGTCTTGAATTTAGCATGGCTCAGACAGTCACTCA
GTCTCAACCAGAGATGTCTGTGCAGGAGGCAGAGACCGTGACCCTGAGCTGCACATA
TGACACCAGTGAGAGTGATTATTATTTATTCTGGTACAAGCAGCCTCCCAG3′
Paired amino acid sequence in TRGV9-CDR3 region
(SEQ ID NO: 58
ALFIQELGKKIKV
(SEQ ID NO: 60)
ALWDGPYYKKL
(SEQ ID NO: 62)
ALWDIPPGQELGKKIKV
(SEQ ID NO: 64)
ALWEAQELGKKIKV
(SEQ ID NO: 66)
ALWEARQELGKKIKV
(SEQ ID NO: 68)
ALWEGTRGQELGKKIKV
(SEQ ID NO: 70)
ALWEVGDQELGKKIKV
(SEQ ID NO: 72)
ALWEVHSELGKKIKV
(SEQ ID NO: 72)
ALWEVHSELGKKIKV
(SEQ ID NO: 74)
ALWEVLELGKKIKV
(SEQ ID NO: 76)
ALWEVLVGELGKKIKV
(SEQ ID NO: 78)
ALWEVPELGKKIKV
(SEQ ID NO: 80)
ALWEVQELGKKIKV
(SEQ ID NO: 80)
ALWEVQELGKKIKV
(SEQ ID NO: 80)
ALWEVQELGKKIKV
(SEQ ID NO: 80)
ALWEVQELGKKIKV
(SEQ ID NO: 80)
ALWEVQELGKKIKV
(SEQ ID NO: 80)
ALWEVQELGKKIKV
(SEQ ID NO: 87)
ALWEVRELGKKIKV
(SEQ ID NO: 87)
ALWEVRELGKKIKV
(SEQ ID NO: 90)
ALWEVRKQELGKKIKV
(SEQ ID NO: 92)
ALWEVTHNRQELGKKIKV
(SEQ ID NO: 94)
ALWGGAAGAYYKKL
(SEQ ID NO: 96)
ALWGGELGKKIKV
(SEQ ID NO: 98)
ALWVQELGKKIKV
(SEQ ID NO: 100)
ALWEAHQELGKKIKV
(SEQ ID NO: 102)
ALWEANKKL
(SEQ ID NO: 104)
ALWEAQELGKKIKV
(SEQ ID NO: 106)
ALWEATGLGKKIKV
(SEQ ID NO: 108)
ALWEDLELGKKIKV
(SEQ ID NO: 110)
ALWEKEELGKKIKV
(SEQ ID NO: 112)
ALWEKELGKKIKV
(SEQ ID NO: 114)
ALWEMTQELGKKIKV
(SEQ ID NO: 116)
ALWEPQELGKKIKV
(SEQ ID NO: 118)
ALWESKELGKKIKV
(SEQ ID NO: 120)
ALWEVGELGKKIKV
(SEQ ID NO: 122)
ALWEVHKLGKKIKV
(SEQ ID NO: 124)
ALWEVKELGKKIKV
(SEQ ID NO: 126)
ALWEVLQQELGKKIKV
(SEQ ID NO: 128)
ALWEVPVLGKKIKV
(SEQ ID NO: 80)
ALWEVQELGKKIKV
(SEQ ID NO: 80)
ALWEVQELGKKIKV
(SEQ ID NO: 80)
ALWEVQELGKKIKV
(SEQ ID NO: 87)
ALWEVRELGKKIKV
(SEQ ID NO: 87)
ALWEVRELGKKIKV
(SEQ ID NO: 135)
ALWEVRQELGKKIKV
(SEQ ID NO: 137)
ALWEVRVQELGKKIKV
(SEQ ID NO: 139)
ALWEVTELGKKIKV
(SEQ ID NO: 141)
ALWGRELGKKIKV
Paired amino acid sequence in TRDV2-CDR3 region
(SEQ ID NO: 59
ACDVLGDTEGRLI
(SEQ ID NO: 61)
ACDTVFTGGYSSWDTRQMF
(SEQ ID NO: 63)
ACDTLGETSSWDTRQMF
(SEQ ID NO: 65)
ACDSGGYSSWDTRQMF
(SEQ ID NO: 67)
ACDTLFPGGSATDKLI
(SEQ ID NO: 69)
ACDTVGAHTDKLI
(SEQ ID NO: 71)
ACDPLNTGGSFSLYTDKLI
(SEQ ID NO: 73)
ACDTGGFRSSWDTRQMF
(SEQ ID NO: 73)
ACDTGGFRSSWDTRQMF
(SEQ ID NO: 75)
ACDTVGMGIRLGDKLI
(SEQ ID NO: 77)
ACDILGINTDKLI
(SEQ ID NO: 79)
ACERLGDYVPDKLI
(SEQ ID NO: 81)
ACDRLLGDTDKLI
(SEQ ID NO: 82)
ACDTVAPRIGGLKYTDKLI
(SEQ ID NO: 83)
ACDTVGGPYTDKLI
(SEQ ID NO: 84)
ACDTVGGTAQ
(SEQ ID NO: 85)
ACDTVSGGSTPTWYTDKLI
(SEQ ID NO: 86)
ACDTVSIFTGDTTDKLI
(SEQ ID NO: 88)
ACDTILIFSPTGGDTDKLI
(SEQ ID NO: 89)
ACVPLGDWTDKLI
(SEQ ID NO: 91)
ACDTLGDDFDKLI
(SEQ ID NO: 93)
ACDTLLGTEAWDTRQMF
(SEQ ID NO: 95)
ACDGKTTDTDKLI
(SEQ ID NO: 97)
ACDLLGDTRYTDKLI
(SEQ ID NO: 91)
ACVGITGDTDKLI
(SEQ ID NO: 101)
ACDSLGDSVDKLI
(SEQ ID NO: 103)
ACDLLRGAGGQIDKLI
(SEQ ID NO: 105)
ACDTVGGAFDTDKLI
(SEQ ID NO: 107)
ACDMGDTRSWDTRQMF
(SEQ ID NO: 109)
ACDTVSWGKNTDKLI
(SEQ ID NO: 111)
ACDTGDWGSSWDTRQMF
(SEQ ID NO: 113)
ACDILDSTGGTDLTAQLF
(SEQ ID NO: 115)
ACDTVRNTGGYAFAGIDKLI
(SEQ ID NO: 117)
ACDKVLGDSSWDTRQMF
(SEQ ID NO: 119)
ACEGLGATQSSWDTRQMF
(SEQ ID NO: 121)
ACDKLLGDNELI
(SEQ ID NO: 123)
ACDSLLGKGTDKLI
(SEQ ID NO: 125)
ACDTLRGSADKLI
(SEQ ID NO: 127)
ACDTVPARHTDKLI
(SEQ ID NO: 129)
ACDTADRSSYTDKLI
(SEQ ID NO: 130)
ACDTLLGDPSSSWDTRQMF
(SEQ ID NO: 131)
ACDTLSGGYARTDKLI
(SEQ ID NO: 132)
ACDTVGILGDTGLGLI
(SEQ ID NO: 133)
ACDTIVSGYDGYDKLI
(SEQ ID NO: 134)
ACSILGDKTSDKLI
(SEQ ID NO: 136)
ACDTVSQRGGYSDKLI
(SEQ ID NO: 138)
ACDPLERVGGPANTDKLI
(SEQ ID NO: 140)
ACDVLGDTGDDKLI
(SEQ ID NO: 142)
ACDTVGSNTDKLI
(SEQ ID NO: 143)
ACDVLGDTEADKLI
(SEQ ID NO: 145)
ACDPLEGAGGHNTDKLI
(SEQ ID NO: 146)
GILGFVFTL
TRGV1-3
External Primer:
(SEQ ID NO: 147)
GCAGCTGGAGCAAACTG
Internal Primer:
(SEQ ID NO: 148)
CTGAATTATCGGTCACCAG
TRGV4
External Primer:
(SEQ ID NO: 149)
CAAATATCCTGTAAAGTTTTCATC
Internal Primer:
(SEQ ID NO: 150)
GTTTAGAGTTTCTATTATATGTCCTTGCAAC
TRGV5
External Primer:
(SEQ ID NO: 151)
GATATCTCAGGATCAGCTCTCC
Internal Primer:
(SEQ ID NO: 152)
TACCCGAAGACCAAACAAGAC
TRGV6
External Primer:
(SEQ ID NO: 153)
TCACCTCTGGGGTCATATG
Internal Primer:
(SEQ ID NO: 154)
AGAGGAAAGGAAATACGGC
TRGV7
External Primer:
(SEQ ID NO: 155)
CAACTTGGAAGAAAGAATAATGTC
Internal Primer:
(SEQ ID NO: 156)
CACCAAGCTAGAGGGGTC
TRGC (reverse)
External Primer:
(SEQ ID NO: 157)
CTTTTCTTTCCAATACACCC
Internal Primer:
(SEQ ID NO: 158)
TCDGGAAAGAACTTTTCAAGG
TRDV1
External Primer:
(SEQ ID NO: 159)
ACCCAAATGTTGCATCAG
Internal Primer:
(SEQ ID NO: 160)
GTCTCTGACAATCCAAGAAGG
TRDV2
External Primer:
(SEQ ID NO: 161)
TCTGTGCAGGTGGCAG
Internal Primer:
(SEQ ID NO: 162)
CCCTGGACTGCACCTATG
TRDV4
External Primer:
(SEQ ID NO: 163)
TGTATATTTGGAACCAGTTGC
Internal Primer:
(SEQ ID NO: 164)
GATCCTGCCTCCTTCTACTG
TRDV5
External Primer:
(SEQ ID NO: 165)
CATCACGCTGACCCAG
Internal Primer:
(SEQ ID NO: 166)
GCTCCACTGACCAGACAG
TRDV6/TRAV15
External Primer:
(SEQ ID NO: 167)
CASCTTYTTAGTGGAGAGATGG
Internal Primer:
(SEQ ID NO: 168)
AYTCTGTAGTCTTCCAGAAATCAC
TRDV7/TRAV13
External Primer:
(SEQ ID NO: 169)
TCCTTGGTTCTGCAGG
Internal Primer:
(SEQ ID NO: 170)
TGCAGGAGGGGGAGA
TRDV8/TRAV14
External Primer:
(SEQ ID NO: 171)
GCAGCAGGTGAGACAAAG
Internal Primer:
(SEQ ID NO: 172)
CTCTGACAGTCTGGGAAGG
TRDV9/TRAV6-1/6-2
External Primer:
(SEQ ID NO: 173)
CAGATGCAAGGTCAAGTGAC
Internal Primer:
(SEQ ID NO: 174)
GGAGAAGGTCCACAGCTC
TRDV9/TRAV6-3/6-4
External Primer:
(SEQ ID NO: 175)
AAGGTCCACAGCTCCTTC
Internal Primer:
(SEQ ID NO: 176)
CAACTGCCAACAACAAGG
TRDV9/TRAV6-5/6-7
External Primer:
(SEQ ID NO: 177)
GTTCTGGTATGTGCAGTATCC
Internal Primer:
(SEQ ID NO: 178)
TCCTTCCACTTGCAGAAAG
TRDV10/TRAV4
External Primer:
(SEQ ID NO: 179)
TCTGSTCTGAGATGCAATTTT
Internal Primer:
(SEQ ID NO: 180)
GGITIMAGGAACAAAGGAGAAT
TRDV11/TRAV16
External Primer:
(SEQ ID NO: 181)
GTACAAGCAAACAGCAAGTG
Internal Primer:
(SEQ ID NO: 182)
ATTATTCTCTGAACTTTCAGAAGC
TRDV12/TRAV21
External Primer:
(SEQ ID NO: 183)
GTGCACTTGCCTTGTAGC
Internal Primer:
(SEQ ID NO: 184)
AATAGTATGGCTTTCCTGGC
TRDC (reverse)
External Primer:
(SEQ ID NO: 185)
TGAAAGAATTTTGCATATGGTTC
Internal Primer:
(SEQ ID NO: 186)
GAGATGACTATAGCAGGGTCG

1.-10. (canceled)

11. A method for constructing a TCRαβ and/or TCRγδ library in an expression vector, comprising:
(a) synthesizing multiple pairs of TRGV and TRDV DNA fragments or TRAV and TRBV DNA fragments with a 15-25 bp overlap to the vector sequence based on the amplified sequence of the TRGV/TRDV or TRAV/TRBV pairings, respectively, and
(b) performing a two- or three-way ligation with a linearized expression vector.

12. (canceled)

13. The method of claim 11, wherein the ligation in step (b) is performed using Gibson Assembly Cloning techniques.

14. The method of claim 11, wherein the TCRαβ and/or TCRγδ library is constructed after a single-cell amplification and synthesized paired TRGV/TRDV or TRAV/TRBV receptors based on the sequence data.

15. The method of claim 13, wherein said Gibson Assembly Cloning techniques are optimized to clone synthesized TRGV/TRAV and TRDV/TRBV DNA fragments using g-blocks or other synthesized DNA fragments.

16.-18. (canceled)

19. A method for rapid cloning of TCRαβ and/or TCRγδ chains using the TCRαβ and/or TCRγδ library constructed according to the method of claim 11, said cloning method comprising substituting CDR3 regions of the existing clones in the TCRαβ and/or TCRγδ library with (i) products of RT-PCR performed using a primer mixture on a single T cell to obtain paired αβ or γδ TCR CDR3 DNA sequences comprising a partial variable (V) region, CDR3 region, and a partial constant (C) region and (ii) a linker DNA for overlap extension of PCR cloning.

20. The method of claim 19, wherein said T cell is a human or a mouse αβ or γδ T cell.

21. The method of claim 19, comprising sorting of single T cells prior to RT-PCR.

22. The method of claim 21, wherein T cells are not stimulated prior to sorting.

23. The method of claim 19, wherein the primer mixture comprises sense primers comprising T-cell receptor gamma variable (TRGV) and/or T cell receptor delta variable (TRDV) and antisense primers comprising T-cell receptor gamma constant (TRGC) and/or T-cell receptor delta constant (TRDC) sequences.

24. The method of claim 23, wherein the primer mixture comprises 9 TRGV and 8 TRDV sense primers and single TRGC and TRDC antisense primers.

25. The method of claim 23, wherein the primer mixture comprises 5 external and 5 internal TRGV and 13 external and 13 internal TRDV sense primers and single TRGC and TRDC antisense primers.

26. The method of claim 23, wherein the TRGV and/or TRDV sense primers and the TRGC and/or TRDC antisense primers are selected from the primers listed in Table 1.

27. The method of claim 23, wherein the TRGV and/or TRDV sense primers and the TRGC and/or TRDC antisense primers are selected from the primers listed in Table 6.

28. The method of claim 19, wherein said linkers are overlapping with the non-variant sequences of the TCRα/γ and TCRβ/δ single cell RT-PCR products.

29. The method of claim 19, wherein said linker sequences are selected from those listed in Table 3.

30. The method of claim 19, wherein the resulting TCRαβ and/or TCRγδ chains with CDR3 substitutions are used for T cell-mediated immunotherapy.

31. A TCRαβ and/or TCRγδ library constructed using the method of claim 11.

32. A host cell comprising the TCRαβ and/or TCRγδ library construct of claim 31.

33. (canceled)

34. A method for cloning a B cell receptor (BCR) from a single B cell, wherein said method comprises:
(a) performing RT-PCR with a primer mixture on a single B cell to obtain paired IgH or Igκ or Igλ CDR3 DNA sequences comprising a partial variable (V) region, CDR3 region, and a partial constant (C) region,
(b) optionally sequencing the RT-PCR product obtained in step (a), and
(c) cloning the IgH or Igκ or IgA CDR3 DNA sequences obtained in step (a) into a corresponding BCR library.

35. (canceled)

36. The method of claim 34, comprising sorting of single B cells prior to step (a).
